(19) United States
(12) Patent Application Publication
(10) Pub. No.: US 2022/0395584 A1
(43) Pub. Date: Dec. 15, 2022

(54) REVERSIBLE COATING OF CHITOSAN-NUCLEIC ACID NANOPARTICLES AND METHODS OF THEIR USE

(71) Applicant: ENGENE, INC, Montreal (CA)

(72) Inventors: Rajesh Krishnan Gopalakrishna PANICKER, Montreal (CA); Daniel VEILLEUX, Montreal (CA); Pei Lian MA, Montreal (CA); Natalie Chin Mun TAM, Montreal (CA); Carlos FLEET, Montreal (CA); Anthony CHEUNG, Montreal (CA)

(21) Appl. No.: 17/438,921

(22) PCT Filed: Mar. 13, 2020
(86) PCT No.: PCT/IB2020/000175

§ 371 Date: Sep. 13, 2021

Related U.S. Application Data
(60) Provisional application No. 62/818,425, filed on Mar. 14, 2019.
(60) Provisional application No. 62/923,403, filed on Oct. 18, 2019.
(60) Provisional application No. 62/924,131, filed on Oct. 21, 2019.

(51) Int. Cl. A61K 48/00; A61K 47/69; A61K 47/60; A61K 47/61

(52) U.S. Cl. CPC A61K 48/0041; A61K 47/6939; A61K 47/60; A61K 47/61; A61K 48/0075

(57) ABSTRACT

Chitosan-nucleic acid polyplex compositions containing a reversibly bound polymer coat comprising linear block copolymers with a polyanionic anchor region and at least one polyethylene glycol tail region are described herein. In some cases, the compositions exhibit improved stability and/or mucosal diffusion as compared to uncoated particles. In some cases, the reversibly bound polymer coat does not interfere with, or enhances, transfection of target cells or tissues as compared to uncoated particles.

### CROSS-REFERENCES TO RELATED APPLICATIONS

This application claims the benefit of priority to U.S. Provisional Application Nos. 62/818,425, filed Mar. 14, 2019; 62/923,403, filed Oct. 18, 2019; and 62/924,131, filed Oct. 21, 2019, the contents of which are hereby incorporated by reference in their entirety and for all purposes.

### BACKGROUND

Chitosan is the deacetylated form of chitin. Chitosan is a non-toxic cationic polymer of N-acteyl-D-glucosamine and D-glucosamine. Chitosan can form a complex with nucleic acid and, as a biocompatible non-toxic polysaccharide, has been used as a DNA delivery vehicle to transfect cells. Much interest has been focused on using chitosan in non-viral delivery of nucleic acid due to the complexities and potential toxicity of the viral vector.

A number of chitosan/DNA complexes, including complexes between modified chitosan and nucleic acids, have been examined in an attempt to identify compositions well-suited for gene transfection. See, e.g., WO 2010/088565; WO 2008/082282. These complexes have been found to vary in important physico-chemical and biological properties, among other properties, solubility, propensity for aggregation, complex stability, particle size, ability to release DNA, and transfection efficiency.

Chitosan-nucleic acid polyplexes with improved transfection efficiency have been developed by functionalizing the chitosan backbone with arginine and hydrophilic polyols. See, e.g., U.S. Pat. Nos. 10,046,066 and 9,623,112. In particular, these polyplexes have demonstrated remarkable stability in the gut microenvironment, which presents unique challenges in comparison with systemic circulation due to significant pH variations, enzymatic activity and complex mucosal interactions.

The mucosal barrier represents a particular challenge for nanoparticle delivery systems, owing to the dense network of mucin fibers that efficiently trap foreign particulates through adhesive and steric interactions, followed by rapid clearance. Indeed, for oral drug delivery in particular, the intestinal mucosa can turn over in as little as fifty (50) minutes, resulting in very efficient clearance of administered particles. See, e.g., Lehr et al., Int'l J. Pharm. 70:235-40 (1991). Mucin fibers contain highly glycosylated segments with high affinity for positively-charged particles, as well as periodic hydrophobic domains that can bind hydrophobic materials with high avidity, including many commonly used drug delivery materials such as poly-(lactic-co-glycolic acid) (PLGA). These complex charge interactions coupled with steric hindrance have proven difficult for nanoparticle drug delivery forms to overcome without some form of shielding. Conversely, however, conventional shielding approaches often yield a nanoparticle having greatly reduced transfection capability.

Polyethylene glycol (PEG) has emerged as the primary constituent in a wide range of shielding strategies, based at least initially on its demonstrated ability to increase mucoadhesion via interpenetrating network effects, thereby prolonging drug delivery. See, e.g. Huckaby and Lai, Advanced Drug Delivery Reviews 124:125-39 (2018). Unfortunately, however, these mucoadhesive strategies do not work for therapeutics requiring intracellular delivery such as nucleic acids, since enhanced mucoadhesion prevents the particles from reaching the underlying epithelium or other associated target tissues. Paradoxically, perhaps, PEG coatings have also found use in muco-inert approaches attempting to enhance particle diffusion. Notably, however, recent findings and conclusions with these types of systems have underscored the importance of high PEG grafting density on minimizing mucin affinity and enhancing mucus penetration. Id., Wang and Lai, Angew Chem 47:9726-9729 (2008). These exceptionally dense and covalently-bound PEGylation strategies are problematic for efficient gene transfection.

Thus, there remains a need for new compositions and methods for gene transfer in vivo with improved mucosal penetration properties.

### SUMMARY

Provided herein are compositions and methods for reducing mucoadhesion and enhancing mucus penetration of chitosan-nucleic acid nanoparticles, employing a reversible (i.e. non-covalently bound) polymeric coating comprising a plurality of linear block copolymers having a negatively charged anchor region and at least one (e.g., hydrophilic) non-charged tail region. In preferred embodiments, the linear block copolymer is a diblock or triblock copolymer comprising at least one polyanion anchor region and at least one PEG tail region. In some embodiments, the compositions described herein surprisingly provide reduced mucoadhesion and enhanced mucus penetration at a lower surface coating density than previously contemplated. In some embodiments, the compositions described herein surprisingly provide consistent physical and/or chemical stability despite the presence of the polyanion. In some embodiments, the compositions described herein surprisingly provide consistent and/or comparable transfection efficiency despite the inclusion of the reversible polymer coating. In some embodiments, the compositions described herein are surprisingly stable during long-term (e.g., months) storage in bulk and/or coated or encapsulated form. In some embodiments, the compositions described herein are surprisingly stable when in contact with the mucosal barrier and/or a mucus associated fluid (e.g., urine, or gastric or intestinal fluid).

In one aspect the invention provides chitosan compositions comprising a chitosan-derivative nucleic acid nanoparticle (polyplex) in complex with a plurality of polyanion-containing block co-polymers, e.g., linear diblock and/or triblock copolymers each copolymer comprising at least one polyanionic anchor region and at least one hydrophilic (e.g., non-charged) tail region. Typically, the polyplex forms a core and the polyanion-hydrophilic polymer forms an, e.g., at least partial, outer coat. The complex between the polyplex and the polyanion-containing block co-polymer is typically formed by way of a reversible and non-covalent electrostatic interaction between the polyanion anchor region of the polymer and a net-positive charge of the uncoated polyplex. In some cases, the complex is reversible in that all or a portion of the polyanion-hydrophilic polymer can be released from the complex by an increase in ionic strength to reduce the strength of the electrostatic interaction between polyplex and polyanion anchor region of the polymer and/or a decrease in pH to protonate anionic moieties in the polyanion region of the polymer.

Exemplary diblock copolymer molecules useful in the methods and compositions of the present invention are “PEG-PA” copolymer molecules comprising a polyethylene glycol (PEG) tail region and a polyanion (PA) anchor region. Exemplary triblock copolymer molecules useful in the methods and compositions of the present invention are “PEG-PA” copolymer molecules comprising a central PA anchor region flanked by two PEG tail regions [PEG-PA-PEG], or two PA anchor regions flanking a central PEG tail region [PA-PEG-PA].

In some embodiments, the invention provides a complex comprising a chitosan-derivative nanoparticle comprising amino-functionalized chitosan and at least one nucleic acid molecule, wherein the at least one nucleic acid molecule is non-covalently bound to the chitosan-derivative nanoparticle at an amino to phosphorous (N:P) molar ratio of greater than 3:1, thereby forming a derivatized chitosan nucleic acid complex having a positive charge; and a plurality of linear block copolymers non-covalently bound to the chitosan-derivative nanoparticle, wherein the block copolymers comprise at least one polyanion (PA) anchor region and at least one polyethylene glycol (PEG) tail region, and wherein the composition comprises an amino to anion (N:A) molar ratio that is greater than about 1:100 and less than about 10:1.

In some embodiments, the linear block copolymer is a diblock copolymer comprising a PA anchor region and a PEG tail region. In some embodiments, the linear block copolymer is a triblock copolymer comprising a central PA anchor region flanked by two PEG tail regions, or alternatively a central PEG tail region flanked by two PA anchor regions.

In some embodiments, the PA anchor region comprises a polypeptide, wherein the polypeptide is negatively charged. In some embodiments, the PA anchor region of the PEG-PA molecules comprise a carbohydrate, wherein the carbohydrate is negatively charged. In some embodiments, the carbohydrate comprises a plurality of phosphate and/or sulfate moieties. In some embodiments, the carbohydrate comprises a plurality of carboxylate moieties. In some embodiments, the carbohydrate comprises a plurality of carboxylate moieties and a plurality of phosphate and/or sulfate moieties. In some embodiments, the carbohydrate comprises a higher proportion, or number, of carboxylate moieties than phosphate and/or sulfate moieties. In an exemplary embodiment, the carbohydrate is a glycosaminoglycan.

In particular embodiments, the PEG-PA molecules comprise: PEG-polyglutamic acid (PEG-PGA) molecules; PEG-polyaspartic acid (PEG-PAA) molecules; or PEG-hyaluronic acid (PEG-HA) molecules, or a combination thereof.

In some embodiments, the PEG tail region of the PEG-PA molecules comprise a weight average molecular weight (Mw) of from about 500 Da to about 50,000 Da, preferably from about 1,000 Da to about 10,000 Da, more preferably from about 1,500 Da to about 7,500 Da, yet more preferably from about 3,000 Da to about 5,000 Da, most preferably about 5,000 Da. In some embodiments, the PA tail region of the PEG-PA molecules comprise a weight average molecular weight (Mw) of from about 500 Da to about 3,000 Da, more preferably from about 1,000 Da to about 2,500 Da, more preferably about 1,500 Da.

In some embodiments, the N:P molar ratio is greater than about 3:1 and less than about 100:1, more preferably greater than about 5:1 and less than about 50:1, yet more preferably greater than about 5:1 and less than about 30:1, yet more preferably greater than about 5:1 and less than about 20:1, yet more preferably greater than about 5:1 and less than about 10:1. In some cases, the N:P molar ratio is from about 3:1 to about 30:1. In some cases, the N:P molar ratio is from about 3:1 to about 20:1. In some cases, the N:P molar ratio is from about 3:1 to about 10:1. In some cases, the N:P molar ratio is about 7:1.

In some embodiments, the N:A molar ratio is greater than about 1:75 and less than about 8:1, more preferably greater than about 1:50 and less than about 6:1, yet more preferably greater than about 1:25 and less than about 6:1, yet more preferably greater than about 1:10 and less than about 6:1, yet more preferably greater than about 1:5 and less than about 6:1.

In some embodiments, the N:P molar ratio is from about 1:8 to about 8:1, and the P:A molar ratio is from about 0.02 to about 0.2, more preferably wherein the N:A molar ratio is from about 0.1 to about 5, more preferably from about 0.2 to about 2, more preferably from about 0.3 to about 1.5, more preferably from about 0.4 to about 1, yet more preferably wherein the N:P:A ratio is about 7:1:7; about 7:1:12; or about 7:1:17.

In some embodiments, the N:P molar ratio is from about 1:8 to about 30:1, and the P:A molar ratio is from about 1:50 to about 1:5, more preferably wherein the N:A molar ratio is from about 1:10 to about 5, more preferably from about 1:5 to about 2, more preferably from about 1:3 to about 1.5, yest more preferably from about 1:2.5 to about 1. In some embodiments, the N:P molar ratio is from about 1:5 to about 20:1, and the P:A molar ratio is from about 1:50 to about 1:5, more preferably wherein the N:A molar ratio is from about 1:10 to about 5, more preferably from about 1:5 to about 2, more preferably from about 1:3 to about 1.5, yest more preferably from about 1:2.5 to about 1. In some embodiments, the N:P molar ratio is from about 1:2 to about 10:1, and the P:A molar ratio is from about 1:50 to about 1:5, more preferably wherein the N:A molar ratio is from about 1:10 to about 5, more preferably from about 1:5 to about 2, more preferably from about 1:3 to about 1.5, yest more preferably from about 1:2.5 to about 1.

In some embodiments, the N:P molar ratio is from about 1:1 to about 30:1, and the P:A molar ratio is from about 1:50 to about 1:5, more preferably wherein the N:A molar ratio is from about 1:10 to about 5, more preferably from about 1:5 to about 2, more preferably from about 1:3 to about 1.5, yest more preferably from about 1:2.5 to about 1. In some embodiments, the N:P molar ratio is from about 1:1 to about 20:1, and the P:A molar ratio is from about 1:50 to about 1:5, more preferably wherein the N:A molar ratio is from about 1:10 to about 5, more preferably from about 1:5 to about 2, more preferably from about 1:3 to about 1.5, yest more preferably from about 1:2.5 to about 1. In some embodiments, the N:P molar ratio is from about 1:1 to about 15:1, and the P:A molar ratio is from about 1:50 to about 1:5, more preferably wherein the N:A molar ratio is from about 1:10 to about 5, more preferably from about 1:5 to about 2, more preferably from about 1:3 to about 1.5, yest more preferably from about 1:2.5 to about 1. In some embodiments, the N:P molar ratio is from about 1:1 to about 10:1, and the P:A molar ratio is from about 1:50 to about 1:5, more preferably wherein the N:A molar ratio is from about 1:10 to about 5, more preferably from about 1:5 to about 2, more preferably from about 1:3 to about 1.5, yest more preferably from about 1:2.5 to about 1.

In some embodiments, the N:P molar ratio is from about 2:1 to about 30:1, and the P:A molar ratio is from about 1:50 to about 1:5, more preferably wherein the N:A molar ratio is from about 1:10 to about 5, more preferably from about 1:5 to about 2, more preferably from about 1:3 to about 1.5, yest more preferably from about 1:2.5 to about 1. In some embodiments, the N:P molar ratio is from about 2:1 to about 20:1, and the P:A molar ratio is from about 1:50 to about 1:5, more preferably wherein the N:A molar ratio is from about 1:10 to about 5, more preferably from about 1:5 to about 2, more preferably from about 1:3 to about 1.5, yest more preferably from about 1:2.5 to about 1. In some embodiments, the N:P molar ratio is from about 2:1 to about 15:1, and the P:A molar ratio is from about 1:50 to about 1:5, more preferably wherein the N:A molar ratio is from about 1:10 to about 5, more preferably from about 1:5 to about 2, more preferably from about 1:3 to about 1.5, yest more preferably from about 1:2.5 to about 1. In some embodiments, the N:P molar ratio is from about 2:1 to about 10:1, and the P:A molar ratio is from about 1:50 to about 1:5, more preferably wherein the N:A molar ratio is from about 1:10 to about 5, more preferably from about 1:5 to about 2, more preferably from about 1:3 to about 1.5, yest more preferably from about 1:2.5 to about 1.

In some embodiments, the amino-functionalized chitosan is arginine, lysine, or ornithine functionalized, preferably arginine. In some embodiments, the amino-functionalized chitosan-derivative nanoparticle further comprises a polyol. In some embodiments, the amino-functionalized chitosan further comprises a polyol. In some embodiments, the amino-functionalized chitosan-derivative nanoparticle is also functionalized with a polyol. In some embodiments, the amino-functionalized chitosan is also functionalized with a polyol.

In some embodiments, the composition is stable for, or for at least, 1 hr, 24 h, 48 h, 1 week, or 1 or 2 months in fasted state simulated intestinal fluid. In some embodiments, the composition is stable for, or for at least, 1 hr, 24 h, 48 h, 1 week, or 1 or 2 months at 4° C. in an aqueous dispersion, such as a dispersion of the composition in purified water. In some embodiments, the composition is stable for, or for at least, 1 h, 24 h, 48 h, 1 week, or 1 or 2 months at 4° C. in purified water. In some embodiments, the composition is stable for, or for at least, 1 h, 24 h, 48 h, 1 week, or 1 or 2 months at 4° C. in urine. In some embodiments, the composition is stable in that it is substantially free (<10%) of precipitating aggregates in the simulated intestinal fluid and/or aqueous dispersion and/or urine and/or purified water after a specified time, e.g., 24 h, 48 h, 1 week, or 1 or 2 months.

In some embodiments, the composition is stable in the aqueous dispersion after freeze/thaw and/or lyophilization/rehydration. In some embodiments, the composition exhibits a polydispersity index of less than 0.2 after, or after at least, 48 h, 1 week, or 1 or 2 months at 4° C. in the aqueous solution. In some embodiments, the composition is stable in the aqueous dispersion after drying (e.g., lyopholization, spray-drying, evaporation, supercritical drying, spray freeze drying, etc.) and then rehydration.

In some embodiments, the composition is stable for at least 1 hour in mammalian urine. In some embodiments, the composition is stable for at least 1 hour in mammalian urine at room temperature or at 37° C. In some embodiments, the composition exhibits a polydispersity index of less than 0.2 after at least 1 h in the mammalian urine (e.g., at 37° C.).

In some embodiments, the composition transfects cells with a therapeutic nucleic acid. In some embodiments, the therapeutic nucleic acid is transcribed to a therapeutic protein. In some embodiments, the therapeutic nucleic acid inhibits expression of an endogenous protein-encoding gene. In some embodiments, the therapeutic nucleic acid inhibits expression of an endogenous gene.

In some embodiments, the composition further comprises a surfactant, excipient, and/or a storage stability agent. In some embodiments, the storage stability agent is a monosaccharide, a disaccharide, a polysaccharide, or a reduced alcohol thereof, yet more preferably wherein the storage stability agent is selected from trehalose and mannitol. In some embodiments, the surfactant comprises a poloxamer, more preferably wherein the poloxamer is poloxamer 407.

In some embodiments, the at least one nucleic acid comprises RNA. In some embodiments, the at least one nucleic acid comprises DNA.

### BRIEF DESCRIPTION OF THE DRAWINGS

FIG. 1 illustrates a polyplex:polymer composition and a method of making the composition.

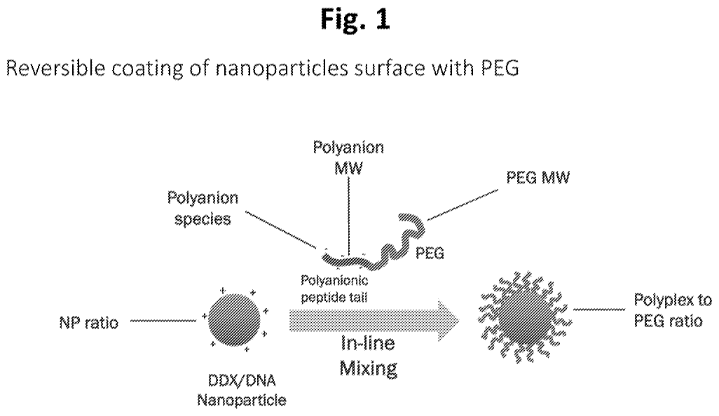

FIG. 2 illustrates an inverse relationship between zeta potential and degree of PEGylation for certain polyplex:polymer compositions described herein. As the molar ratio of polyanion-PEG (A) to amino-functionalized chitosan (N) increases, then the zeta potential decreases to reach a near neutral to slightly negative value at higher PEG density.

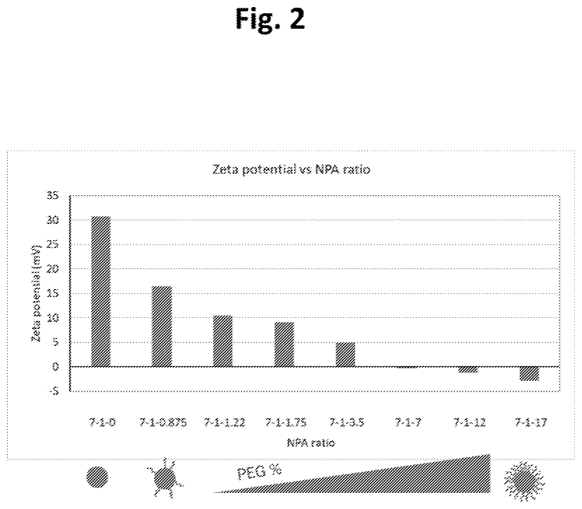

FIG. 3 illustrates the stability of polyplex:polymer compositions after freeze thaw. Polyplexes PEGylated at the tested ratios of [N+] to [A−] remained stable after freeze thaw.

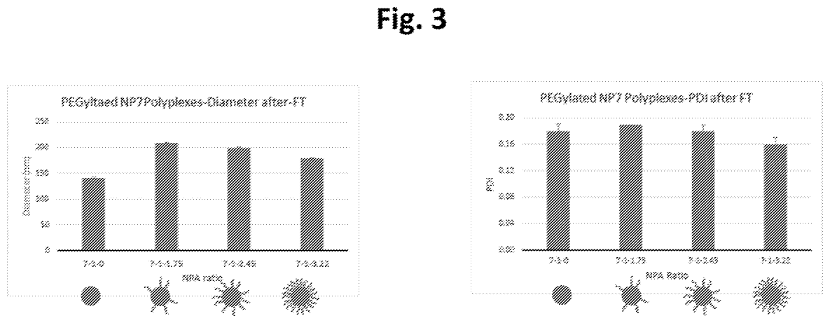

FIG. 4 illustrates stability of polyplex:polymer compositions in simulated intestinal fluid at different volume:volume ratios. FaSSIF-V2=Fasted State Simulated Intestinal Fluid V2.

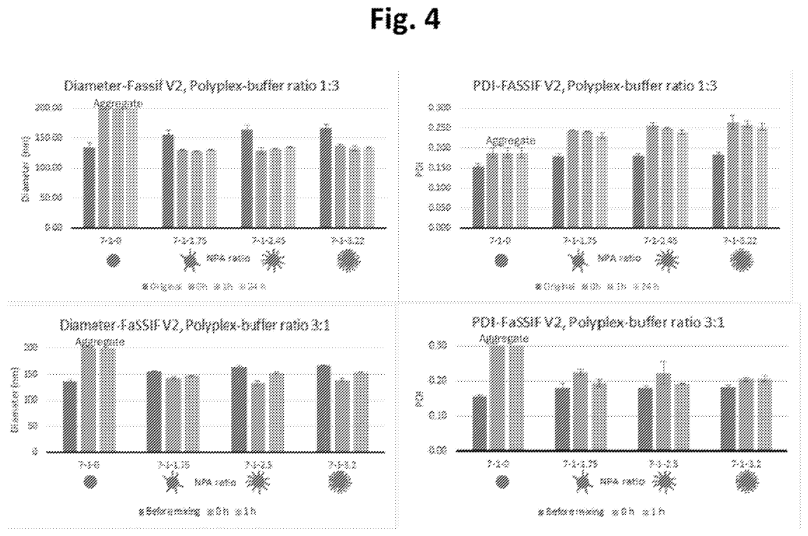

FIG. 5 illustrates the ability of polyplex:polymer compositions to retain complexed nucleic acid in simulated intestinal fluids. FaSSIF-V1=Fasted State Simulated Intestinal Fluid V1. FaSSIF-V2=Fasted State Simulated Intestinal Fluid V2. PP=polyplex.

FIG. 6 shows in vitro transfection with PEGylated DD-chitosan-nucleic acid polyplexes.

FIG. 7 illustrates a method and results of a mucus aggregration assay against polyplex:polymer compositions described herein using fluorescence microscopy.

FIG. 8 illustrates a method of performing a mucus penetration assay against polyplex:polymer compositions described herein using a transwell diffusion assay.

FIG. 9 illustrates results of a transwell diffusion assay of polyplex:polymer compositions described herein.

FIG. 10 illustrates results of a transwell diffusion assay of polyplex:polymer compositions described herein in the presence of a poloxamer 407.

FIG. 11 illustrates a scaleable method of making a polyplex:polymer composition described herein.

FIG. 12 illustrates the stability of polyplex:polymer composition described herein under lyopholization and rehydration, and high concentration conditions.

FIG. 13 illustrates the stability of freeze-dried PEGylated polyplex at room temperature and 4° C. up to 4 weeks.

FIG. 14 illustrates the stability of a polyplex:polymer composition described herein, lyophilized in absence of excipients and stored for up to 4 weeks at 4° C.

FIG. 15 illustrates the stability of a polyplex:polymer composition described herein when administered to a mouse bladder and recovered in subsequently collected urine.

FIG. 16 illustrates markedly improved in vivo gene delivery of a polyplex:polymer composition described herein when delivered by intracolonic instillation (ICI), as measured by transgene mRNA expression. The administered polyplex formulation contained 150 μg/mL nucleic acid and was administered as 3×150 μL intracolonic instillations; colon sections were harvested at 24 h post-administration and cell lysates were used to quantify human PD-L1-Fc mRNA.

FIG. 17 illustrates markedly improved in vivo gene delivery of a polyplex:polymer composition described herein when delivered by intracolonic instillation (ICI), as measured by transgene protein expression. The administered polyplex formulation contained 150 μg/mL nucleic acid and was administered as 3×150 μL intracolonic instillations; colon sections were harvested at 24 h post-administration and protein lysates were used to quantify human PD-L1-Fc protein using a custom-made Mesoscale Discovery immunoassay.

FIG. 18 illustrates markedly improved in vivo gene delivery of a polyplex:polymer composition described herein when delivered by intracolonic instillation (ICI), as measured by transgene protein expression. The administered polyplex formulation contained 1000 μg/mL nucleic acid and was administered as 3×150 μL intracolonic instillations; colon sections were harvested at 24 h post-administration and protein lysates were used to quantify human PD-L1-Fc protein using a custom-made Mesoscale Discovery immunoassay.

FIG. 19 illustrates the improvement in % supercoil DNA content of PEGylated DDX polyplexes as compared to non-PEGylated DDX polyplexes.

FIG. 20 illustrates storage stability of PEGylated DDX polyplexes.

FIG. 21 illustrates the rehydrateability of PEGylated and non-PEGylated DDX polyplex formulations. PEGylated DDX polyplexes were able to be stably rehydrated at higher final concentrations (10 mg/mL) as compared to non-PEGylated DDX polyplexes (2 mg/mL).

FIG. 22 illustrates stability of the indicated DDX polyplex formulations when incubated in the mammalian bladder.

FIG. 23 illustrates filterability of the indicated DDX polyplex formulations at the indicated conditions when tested at small-scale.

FIG. 24 illustrates filterability of the indicated DDX polyplex formulations at the indicated conditions when tested at mid-scale.

FIG. 25 illustrates stability of the specified DDX polyplex formulations after freeze thaw (FT) and lyopholization/rehydration (FD) as indicated by nanoparticle size (nm), zeta potential (mV) and % supercoil content (% SC).

FIG. 26 illustrates in vivo transfection of dog small intestine by delivery of PEGylated dually derivatized (DDX) chitosan DNA polyplexes directly to the small intestine.

FIG. 27 illustrates a significant increase in zeta potential (my) as aqueous polyplex formulation is mixed with low pH acetate buffer in a ratio sufficient to lower the pH to below the pKa of the PEG-polyglutamate polymer (˜4.25), indicating uncoating of the PEGylated polyplexes at pH below 4.

FIG. 28 illustrates particle size of reversibly PEGylated polyplexes formed at various N:P:A ratios as indicated. Different polyglutamate (PLE) polyanion anchor regions were tested: PLE5 (5 glutamate polyglutamate region), PLE10 (10 glutamate polyglutamate region), and PLE25 (25 glutamate polyglutamate region).

FIG. 29 illustrates the pH of an aqueous dispersion of the indicated reversibly PEGylated polyplexes.

FIG. 30 illustrates results of zeta potential measurements of the indicated indicated reversibly PEGylated polyplexes.

FIG. 31 illustrates results of polyaspartic acid (PAA) challenge of reversibly PEGylated polyplexes. Nucleic acid release was monitored as a function of PAA concentration. Different polyglutamate (PLE) polyanion anchor regions of PEG-PLE were tested: PLE5 (5 glutamate polyglutamate region), PLE10 (10 glutamate polyglutamate region), and PLE25 (25 glutamate polyglutamate region) at different NPA ratios.

FIG. 32 illustrates the levels of PAA required for DNA release of PEGylated polyplexes. Nucleic acid in PEGylated polyplexes made using PEG-PLE25 was more loosely bound than nucleic acid in polyplexes made with PEG-PLE5 or PEG-PLE10.

FIG. 33 illustrates the effects of fasted state simulated intestinal fluid (FaSSIF v2) on the particle size and polydispersity index (PDI) of the indicated PEGylated polyplexes.

FIG. 34 illustrates the effects of fasted state simulated intestinal fluid (FaSSIF v2) on the zeta potential of the particles and the pH of the resulting aqueous dispersion of the indicated PEGylated polyplexes.

FIG. 35 illustrates stability of PEGylated polyplexes (PLE10, PLD10, and PLD50) after freeze thaw as measured by particle size. PEG-PLD50 polyplexes at an N:P:A of 7:1:30 exhibited increased particle size after freeze thaw.

FIG. 36 illustrates stability of PEGylated polyplexes (PLE10, PLD10, and PLD50) after freeze thaw as measured by particle size (top), and zeta potential (middle). PEG-PLD50 polyplexes at an N:P:A of 7:1:30 exhibited increased particle size and lower zeta potential after freeze thaw. The indicated polyplexes exhibited increasing pH as an aqueous dispersion as the P:A ratio decreased (A increased) and as the number of anionic subunits increased (bottom).

FIG. 37 illustrates results of analyzing reversibly PEGylated polyplexes by agarose gel electrophoresis to detect uncomplexed nucleic acid.

FIG. 38 illustrates a schematic representation of expected behavior of reversibly PEGylated (top row) and covalently PEGylated polyplexes (bottom row) at different pH.

FIG. 39 illustrates solution behavior of reversibly PEGylated polyplex (top row) and two different covalently PEGylated polyplexes (middle and bottom row) at two pH 2 and 6 and in response to polyaspartic acid (PAA) challenge.

FIG. 40 illustrates transfection efficiency of reversibly PEGylated polyplexes in comparison to unPEGylated polyplex.

FIG. 41 illustrates transfection efficiency of reversibly PEGylated polyplexes in comparison to covalently PEGylated polyplex.

FIG. 42 illustrates transfection potency of reversibly PEGylated polyplexe made in a one-step or a two-step method after intracolonic delivery into mice

FIG. 43 illustrates transfection potency of reversibly PEGylated polyplexes made in a one-step or a two-step method after intravesicular administration to mice.

### DETAILED DESCRIPTION

### 1. Definitions

Unless otherwise defined, all terms of art, notations and other scientific terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a difference over what is generally understood in the art. The techniques and procedures described or referenced herein are generally well understood and commonly employed using conventional methodologies by those skilled in the art, such as, for example, the widely utilized molecular cloning methodologies described in Sambrook et al., Molecular Cloning: A Laboratory Manual 2nd ed. (1989) Cold Spring Harbor Laboratory Press, Cold Spring Harbor, N.Y. As appropriate, procedures involving the use of commercially available kits and reagents are generally carried out in accordance with manufacturer defined protocols and/or parameters unless otherwise noted.

As used herein, the singular forms “a,” “an,” and “the” include the plural referents unless the context clearly indicates otherwise.

The term “about” indicates and encompasses an indicated value and a range above and below that value. In certain embodiments, the term “about” indicates the designated value±10%, ±5%, or ±1%. In certain embodiments, where indicated, the term “about” indicates the designated value±one standard deviation of that value.

The term “combinations thereof” includes every possible combination of elements to which the term refers.

“Treating” or “treatment” of any disease or disorder refers, in certain embodiments, to ameliorating a disease or disorder that exists in a subject. In another embodiment, “treating” or “treatment” includes ameliorating at least one physical parameter, which may be indiscernible by the subject. In yet another embodiment, “treating” or “treatment” includes modulating the disease or disorder, either physically (e.g., stabilization of a discernible symptom) or physiologically (e.g., stabilization of a physical parameter) or both. In yet another embodiment, “treating” or “treatment” includes delaying or preventing the onset of the disease or disorder.

As used herein, the term “therapeutically effective amount” or “effective amount” refers to an amount of an antibody or composition that when administered to a subject is effective to treat a disease or disorder.

As used herein, the term “subject” means a mammalian subject. Exemplary subjects include, but are not limited to humans, monkeys, dogs, cats, mice, rats, cows, horses, camels, avians, goats, and sheep. In certain embodiments, the subject is a human. In some embodiments, the subject has cancer, an autoimmune disease or condition, and/or an infection that can be treated with an antibody provided herein. In some embodiments, the subject is a human that is suspected to have cancer, an autoimmune disease or condition, and/or an infection.

“Chitosan” is a partially or entirely deacetylated form of chitin, a polymer of N-acetylglucosamine. Chitosans with any degree of deacetylation greater than 50% are used in the present invention.

Chitosan may be derivatized by functionalizing free amino groups at the sites of deacetylation. The derivatized chitosans described herein have a number of properties which are advantageous for a nucleic acid delivery vehicle including: they effectively bind and complex the negatively charged nucleic acids, they can be formed into nanoparticles of a controllable size, they can be taken up by the cells and they can release the nucleic acids at the appropriate time within the cells. Chitosans with any degree of final functionalization between 1% and 50%. (Percent functionalization is determined relative to the number of free amino moieties on the chitosan polymer prior-to or in the absence of functionalization.) The degrees of deacetylation and final functionalization impart a specific charge density to the functionalized chitosan derivative.

A polyol according to the present invention may have a 3, 4, 5, 6, or 7 carbon backbone and may have at least 2 hydroxyl groups. Such polyols, or combinations thereof, may be useful for conjugation to a chitosan backbone, such as a chitosan that has been functionalized with a cationic moiety (e.g., a molecule comprising an amino group such as, lysine, ornithine, a molecule comprising a guanidinium group, arginine, or a combination thereof).

The term “C₂-C₆ alkylene” as used herein refers to a linear or branched divalent hydrocarbon radical optionally containing one or more carbon-carbon multiple bonds. For the avoidance of doubt, the term “C₂-C₆ alkylene” as used herein encompasses divalent radicals of alkanes, alkenes and alkynes.

As used herein, unless otherwise indicated, the term “peptide” and “polypeptide” are used interchangeably.

The term “polypeptide” is used in its broadest sense to refer to conventional polypeptides (i.e., short polypeptides containing L or D-amino acids), as well as peptide equivalents, peptide analogs and peptidomimetics that retain the desired functional activity. Peptide equivalents can differ from conventional peptides by the replacement of one or more amino acids with related organic acids, amino acids or the like, or the substitution or modification of side chains or functional groups.

The term “acidic amino acid” refers to a naturally or non-naturally occurring amino acid that has a side chain that is negatively charged in an aqueous buffer at pH 7. Non-limiting examples of acid amino acids are aspartate and glutamate.

Peptidomimetics may have one or more peptide linkages replaced by an alternative linkage, as is known in the art. Portions or all of the peptide backbone can also be replaced by conformationally constrained cyclic alkyl or aryl substituents to restrict mobility of the functional amino acid sidechains, as is known in the art.

The polypeptides of this invention may be produced by recognized methods, such as recombinant and synthetic methods that are well known in the art. Techniques for the synthesis of peptides are well known and include those described in Merrifield, J. Amer. Chem. Soc. 85:2149-2456 (1963), Atherton, et al., Solid Phase Peptide Synthesis: A Practical Approach, IRL Press (1989), and Merrifield, Science 232:341-347 (1986).

As used herein, “linear polypeptide” refers to a polypeptide that lacks branching groups covalently attached to its constituent amino acid side chains. As used herein, “branched polypeptide” refers to a polypeptide that comprises branching groups covalently attached to its constituent amino acid side chains.

The “final functionalization degree” of cation or polyol as used herein refers to the percentage of cation (e.g., amino) groups on the chitosan backbone functionalized with cation (e.g., amino) or polyol, respectively. Accordingly, “α:β ratio”, “final functionalization degree ratio” (e.g., Arg final functionalization degree: polyol final functionalization degree ratio) and the like may be used interchangeably with the term “molar ratio” or “number ratio.”

Dispersed systems consist of particulate matter, known as the dispersed phase, distributed throughout a continuous medium. A “dispersion” of chitosan nucleic acid polyplexes is a composition comprising hydrated chitosan nucleic acid polyplexes, wherein polyplexes are distributed throughout the medium.

As used herein, a “pre-concentrated” dispersion is one that has not undergone the concentrating process to form a concentrated dispersion.

As used herein, “substantially free” of polyplex precipitate means that the composition is essentially free from particles that can be observed on visual inspection.

As used herein, physiological pH refers to a pH between 6 to 8.

By “chitosan nucleic acid polyplex” or its grammatical equivalents is meant a complex comprising a plurality of chitosan molecules and a plurality of nucleic acid molecules. In a preferred embodiment, the (e.g., dually-) derivatized-chitosan is complexed with said nucleic acid.

The term “polyethylene glycol” (“PEG”) as used herein is intended to mean a polymer of ethylene oxide having repeat units of —(CH2CH2-O)— and the general formula of HO—(CH2CH2-O)n-H.

The term “monomethoxy polyethylene glycol” (“mPEG”) as used herein is intended to mean a polymer of ethylene oxide having repeat units of —(CH2CH2-O)— and the general formula of CH3O—(CH2CH2-O)n-H, for example, a PEG capped at one end with a methoxy group.

### 2. Compositions

Provided herein are chitosan compositions comprising a chitosan-derivative nucleic acid nanoparticle (polyplex) in complex with a diblock and/or triblock copolymer coating, wherein individual polymer molecules comprise a negatively charged anchor region and one or more non-charged hydrophilic tail regions. Exemplary negatively charged anchor regions include polyanionic anchor regions comprising repeating units, wherein repeating units comprise one or more negatively charged subunits, such as one or more acidic amino acids. Exemplary hydrophilic tail regions include but are not limited to, PEG tail regions, and derivatives thereof, polyvinyl alcohol tail regions and derivatives thereof, poly oxazoline tail regions and derivatives thereof, polysarcosine tail regions and derivatives thereof, poly(N-(2-hydroxypropyl)methacrylamide) (pHPMA) tail regions and derivatives thereof, and combinations thereof.

Exemplary polymer molecules useful in the methods and compositions of the present invention are “PEG-PA” polymer molecules comprising a polyethylene glycol (PEG) portion and a polyanion (PA) portion.

2.1. Chitosan

The chitosan component of the chitosan-derivative nucleic acid nanoparticle can be functionalized with a cationic functional group and/or a hydrophilic moiety. Chitosan functionalized with two different functional groups is referred to as dually derivitized chitosan (DD-chitosan). Exemplary DD-chitosans are functionalized with both a hydrophilic moiety (e.g., a polyol) and a cationic functional group (e.g., an amino group). Exemplary chitosan derivatives are also described in, e.g., U.S. 2007/0281904; and U.S. 2016/0235863, which are each incorporated herein by reference.

In one embodiment, the dually derivatized chitosan described herein comprises chitosan having a degree of deacetylation of at least 50%. In one embodiment, the degree of deacetylation is at least 60%, more preferably at least 70%, more preferably at least 80%, more preferably at least 90%, and most preferably at least 95%. In a preferred embodiment, the dually derivatized chitosan described herein comprises chitosan having a degree of deacetylation of at least 98%.

The chitosan derivatives described herein have a range of average molecular weights that are soluble at neutral and physiological pH, and include for the purposes of this invention molecular weights ranging from 3-110 kDa. Embodiments described herein feature lower average molecular weight of derivatized chitosans (<25 kDa, e.g., from about 5 kDa to about 25 kDa), which can have desirable delivery and transfection properties, and are small in size and have favorable solubility. A lower average molecular weight derivatized chitosan is generally more soluble than one with a higher molecular weight, the former thus producing a nucleic acid/chitosan complex that will release more easily the nucleic acid and provide increased transfection of cells. Much literature has been devoted to the optimization of all of these parameters for chitosan based delivery systems.

An ordinarily skilled artisan will recognize that chitosan refers to a plurality of molecules having a structure of Formula I, wherein n is any integer, and each R1 is independently selected from acetyl or hydrogen, wherein the degree of R1 selected from hydrogen is between 50% to 100%. Also, chitosan referred to as having an average molecular weight, e.g., of 3 kD to 110 kD, generally refers to a plurality of chitosan molecules having a weight average molecular weight of, e.g., 3 kD to 110 kD, respectively, wherein each of the chitosan molecules may have different chain lengths (n+2). It is also well-recognized that chitosan referred to as “n-mer chitosan,” does not necessarily comprise chitosan molecules of Formula I, wherein each chitosan molecule has a chain length of n+2. Rather, “n-mer chitosan” as used herein refers a plurality of chitosan molecules, each of which may have different chain lengths, wherein the plurality has an average molecule weight substantially similar to or equal to a chitosan molecule having a chain length of n. For example, 24-mer chitosan may comprise a plurality of chitosan molecules, each having different chain lengths ranging from, e.g., 7-50, but which has a weight average molecular weight substantially similar or equivalent to a chitosan molecule having a chain length of 24.

A dually derivatized chitosan of the invention may also be functionalized with a polyol, or a hydrophilic functional group such as a polyol. Without wishing to be bound by theory, it is hypothesized that functionalization with a hydrophilic group such as a polyol which may help to increase the hydrophilicity of chitosan (including Arg-chitosan) and/or may donate a hydroxyl group. In some embodiments, the hydrophilic functional group of the chitosan-derivative nanoparticles is or comprises gluconic acid. See, e.g., WO 2013/138930. In some embodiments, the hydrophilic functional group of the chitosan-derivative nanoparticles is or comprises glucose. Additionally or alternatively, the hydrophilic functional group can comprise a polyol. See, e.g., U.S. 2016/0235863. Exemplarly polyols for functionalization of chitosan are further described below.

The functionalized chitosan derivatives described herein include dually derivatized-chitosan compounds, e.g., cation-chitosan-polyol compounds. In general, the cation-chitosan-polyol compounds are functionalized with an amino-containing moiety, such as an arginine, lysine, ornithine, or molecule comprising a guanidinium, or a combination thereof. In certain embodiments, the cation-chitosan-polyol compounds have the following structure of Formula I:

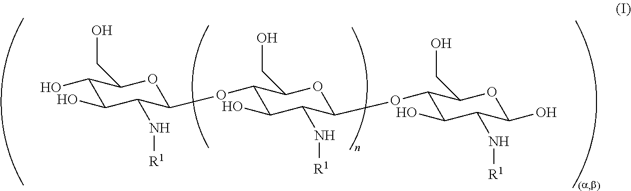

wherein n is an integer of 1 to 650,
α is the final functionalization degree of the cation moiety (e.g., a molecule comprising an amino group such as, lysine, ornithine, a molecule comprising a guanidinium group, arginine, or a combination thereof),
β is the final functionalization degree of polyol; and
each R¹ is independently selected from hydrogen, acetyl, a cation (e.g., arginine), and a polyol.

Preferably, a dually derivatized chitosan of the invention may be functionalized with the cationic amino acid, arginine.

In one embodiment, the chitosan-derivative nanoparticle comprises chitosan coupled with gluconic acid at a final functionalization degree of 1%, 2%, 4%, 7%, 8%, 10%, 15%, 20%, 25%, 30%, or greater. In one embodiment, the chitosan-derivative nanoparticle comprises chitosan coupled with glucose at a final functionalization degree of 1%, 2%, 4%, 7%, 8%, 10%, 15%, 20%, 25%, 30%, or greater. In one embodiment, the chitosan derivative nanoparticle comprises chitosan coupled with a cationic moiety (e.g., arginine) at a final functionalization degree of from about 1% to about 25%. In one embodiment, the chitosan derivative nanoparticle comprises chitosan coupled with a cationic moiety (e.g., arginine) at a final functionalization degree of from about 10% to about 40%.

In one embodiment, the chitosan derivative nanoparticle comprises chitosan coupled with a cationic moiety (e.g., arginine) at a final functionalization degree of from about 10% to about 35%. In one embodiment, the chitosan derivative nanoparticle comprises chitosan coupled with a cationic moiety (e.g., arginine) at a final functionalization degree of from about 20% to about 35%. In one embodiment, the chitosan derivative nanoparticle comprises chitosan coupled with a cationic moiety (e.g., arginine) at a final functionalization degree of from about 25% to about 35%. In one embodiment, the chitosan derivative nanoparticle comprises chitosan coupled with a cationic moiety (e.g., arginine) at a final functionalization degree of from about 25% to about 30%, preferably 28%.

In one embodiment, the chitosan derivative nanoparticle comprises chitosan coupled with a cationic moiety (e.g., arginine) at a final functionalization degree of from about 15% to about 40%. In one embodiment, the chitosan derivative nanoparticle comprises chitosan coupled with a cationic moiety (e.g., arginine) at a final functionalization degree of from about 15% to about 35%. In one embodiment, the chitosan derivative nanoparticle comprises chitosan coupled with a cationic moiety (e.g., arginine) at a final functionalization degree of from about 15% to about 30%. In one embodiment, the chitosan derivative nanoparticle comprises chitosan coupled with a cationic moiety (e.g., arginine) at a final functionalization degree of from about 15% to about 28%.

In one embodiment, the chitosan derivative nanoparticle comprises chitosan coupled with a cationic moiety (e.g., arginine) at a final functionalization degree of from about 10% to about 35%. In one embodiment, the chitosan derivative nanoparticle comprises chitosan coupled with a cationic moiety (e.g., arginine) at a final functionalization degree of from about 10% to about 30%. In one embodiment, the chitosan derivative nanoparticle comprises chitosan coupled with a cationic moiety (e.g., arginine) at a final functionalization degree of from about 10% to about 28%. In one embodiment, the chitosan derivative nanoparticle comprises chitosan coupled with a cationic moiety (e.g., arginine) at a final functionalization degree of about 28%.

In one embodiment, the chitosan-derivative nanoparticle comprises chitosan coupled with gluconic acid at a final functionalization degree of from about 2% to about 30%, from about 5% to about 30%, from about 7.5% to about 30%, from about 5% to about 25%, from about 5% to about 22%, from about 5% to about 20%, from about 5% to about 15%, or from about 5% to about 10%. In one embodiment, the chitosan-derivative nanoparticle comprises chitosan coupled with gluconic acid at a final functionalization degree of from about 7.5% to about 25%, from about 7.5% to about 20%, from about 7.5% to about 15%, or from about 7.5% to about 12%. In one embodiment, the chitosan-derivative nanoparticle comprises chitosan coupled with gluconic acid at a final functionalization degree of about 10%.

In one embodiment, the chitosan-derivative nanoparticle comprises chitosan coupled with hydrophilic polyol at a final functionalization degree of from about 2% to about 30%, from about 5% to about 30%, from about 7.5% to about 30%, from about 5% to about 25%, from about 5% to about 22%, from about 5% to about 20%, from about 5% to about 15%, or from about 5% to about 10%. In one embodiment, the chitosan-derivative nanoparticle comprises chitosan coupled with hydrophilic polyol at a final functionalization degree of from about 7.5% to about 25%, from about 7.5% to about 20%, from about 7.5% to about 15%, or from about 7.5% to about 12%. In one embodiment, the chitosan-derivative nanoparticle comprises chitosan coupled with hydrophilic polyol at a final functionalization degree of about 10%.

In one embodiment, the chitosan-derivative nanoparticle comprises chitosan coupled with glucose at a final functionalization degree of from about 2% to about 30%, from about 5% to about 30%, from about 7.5% to about 30%, from about 5% to about 25%, from about 5% to about 22%, from about 5% to about 20%, from about 5% to about 15%, or from about 5% to about 10%. In one embodiment, the chitosan-derivative nanoparticle comprises chitosan coupled with glucose at a final functionalization degree of from about 7.5% to about 25%, from about 7.5% to about 20%, from about 7.5% to about 15%, or from about 7.5% to about 12%. In one embodiment, the chitosan-derivative nanoparticle comprises chitosan coupled with glucose at a final functionalization degree of about 10%.

In one embodiment, the chitosan-derivative nanoparticle comprises chitosan coupled with cation (e.g., arginine) at a final functionalization degree of from about 2% to about 40% and hydrophilic polyol (e.g., glucose or gluconic acid) at a final functional degree of from about 2% to about 30%. In one embodiment, the chitosan-derivative nanoparticle comprises chitosan coupled with cation (e.g., arginine) at a final functionalization degree of from about 5% to about 40% and hydrophilic polyol (e.g., glucose or gluconic acid) at a final functional degree of from about 5% to about 25%. In one embodiment, the chitosan-derivative nanoparticle comprises chitosan coupled with cation (e.g., arginine) at a final functionalization degree of from about 7.5% to about 40% and hydrophilic polyol (e.g., glucose or gluconic acid) at a final functional degree of from about 7.5% to about 20%. In one embodiment, the chitosan-derivative nanoparticle comprises chitosan coupled with cation (e.g., arginine) at a final functionalization degree of from about 10% to about 40% and hydrophilic polyol (e.g., glucose or gluconic acid) at a final functional degree of from about 7.5% to about 15%, or about 10%.

In one embodiment, the chitosan-derivative nanoparticle comprises chitosan coupled with cation (e.g., arginine) at a final functionalization degree of from about 2% to about 35% and hydrophilic polyol (e.g., glucose or gluconic acid) at a final functional degree of from about 2% to about 30%. In one embodiment, the chitosan-derivative nanoparticle comprises chitosan coupled with cation (e.g., arginine) at a final functionalization degree of from about 5% to about 35% and hydrophilic polyol (e.g., glucose or gluconic acid) at a final functional degree of from about 5% to about 25%. In one embodiment, the chitosan-derivative nanoparticle comprises chitosan coupled with cation (e.g., arginine) at a final functionalization degree of from about 7.5% to about 35% and hydrophilic polyol (e.g., glucose or gluconic acid) at a final functional degree of from about 7.5% to about 20%. In one embodiment, the chitosan-derivative nanoparticle comprises chitosan coupled with cation (e.g., arginine) at a final functionalization degree of from about 10% to about 35% and hydrophilic polyol (e.g., glucose or gluconic acid) at a final functional degree of from about 7.5% to about 15%, or about 10%.

In one embodiment, the chitosan-derivative nanoparticle comprises chitosan coupled with cation (e.g., arginine) at a final functionalization degree of from about 10% to about 30% and hydrophilic polyol (e.g., glucose or gluconic acid) at a final functional degree of from about 2% to about 30%. In one embodiment, the chitosan-derivative nanoparticle comprises chitosan coupled with cation (e.g., arginine) at a final functionalization degree of from about 12% to about 30% and hydrophilic polyol (e.g., glucose or gluconic acid) at a final functional degree of from about 5% to about 25%. In one embodiment, the chitosan-derivative nanoparticle comprises chitosan coupled with cation (e.g., arginine) at a final functionalization degree of from about 14% to about 30% and hydrophilic polyol (e.g., glucose or gluconic acid) at a final functional degree of from about 7.5% to about 20%. In one embodiment, the chitosan-derivative nanoparticle comprises chitosan coupled with cation (e.g., arginine) at a final functionalization degree of from about 15% to about 30% and hydrophilic polyol (e.g., glucose or gluconic acid) at a final functional degree of from about 7.5% to about 15%, or about 10%.

In one embodiment, the chitosan-derivative nanoparticle comprises chitosan coupled with cation (e.g., arginine) at a final functionalization degree of about 25% and hydrophilic polyol (e.g., glucose or gluconic acid) at a final functional degree of from about 7.5% to about 15%. In one embodiment, the chitosan-derivative nanoparticle comprises chitosan coupled with cation (e.g., arginine) at a final functionalization degree of about 28% and hydrophilic polyol (e.g., glucose or gluconic acid) at a final functional degree of from about 7.5% to about 15%. In one embodiment, the chitosan-derivative nanoparticle comprises chitosan coupled with cation (e.g., arginine) at a final functionalization degree of about 25% and hydrophilic polyol (e.g., glucose or gluconic acid) at a final functional degree of from about 5% to about 20%. In one embodiment, the chitosan-derivative nanoparticle comprises chitosan coupled with cation (e.g., arginine) at a final functionalization degree of about 28% and hydrophilic polyol (e.g., glucose or gluconic acid) at a final functional degree of from about 5% to about 20%.

In a preferred embodiment, the chitosan-derivative nanoparticle comprises chitosan coupled with cation (e.g., arginine) at a final functionalization degree of about 14% and hydrophilic polyol (e.g., glucose or gluconic acid) at a final functional degree of about 10%. In a preferred embodiment, the chitosan-derivative nanoparticle comprises chitosan coupled with cation (e.g., arginine) at a final functionalization degree of about 15% and hydrophilic polyol (e.g., glucose or gluconic acid) at a final functional degree of about 12%. In another preferred embodiment, the chitosan-derivative nanoparticle comprises chitosan coupled with arginine at a final functionalization degree of about 14% and glucose at a final functional degree of about 10%. In another preferred embodiment, the chitosan-derivative nanoparticle comprises chitosan coupled with arginine at a final functionalization degree of about 15% and glucose at a final functional degree of about 12%.

In a preferred embodiment, the chitosan-derivative nanoparticle comprises chitosan coupled with cation (e.g., arginine) at a final functionalization degree of about 28% and hydrophilic polyol (e.g., glucose or gluconic acid) at a final functional degree of about 10%. In another preferred embodiment, the chitosan-derivative nanoparticle comprises chitosan coupled with arginine at a final functionalization degree of about 28% and glucose at a final functional degree of about 10%

In some embodiments, where appropriate, DD-chitosan includes DD-chitosan derivatives, e.g., DD chitosan that incorporate an additional functionalization, e.g., DD-chitosan with an attached ligand. “Derivatives” will be understood to include the broad category of chitosan-based polymers comprising covalently modified N-acetyl-D-glucosamine and/or D-glucosamine units, as well as chitosan-based polymers incorporating other units, or attached to other moieties. Derivatives are frequently based on a modification of the hydroxyl group or the amine group of glucosamine, such as done with arginine-functionalized chitosan. Examples of chitosan derivatives include, but are not limited to, trimethylated chitosan, PEGylated chitosan, thiolated chitosan, galactosylated chitosan, alkylated chitosan, PEI-incorporated chitosan, uronic acid modified chitosan, glycol chitosan, and the like. For further teaching on chitosan derivatives, see, for example, pp. 63-74 of “Non-viral Gene Therapy”, K. Taira, K. Kataoka, T. Niidome (editors), Springer-Verlag Tokyo, 2005, ISBN 4-431-25122-7; Zhu et al., Chinese Science Bulletin, December 2007, vol. 52 (23), pp. 3207-3215; and Varma et al., Carbohydrate Polymers 55 (2004) 77-93.

2.2. Chitosan Nucleic Acid Polyplex

The chitosan-derivative nanoparticle compositions generally contain at least one nucleic acid molecule, and preferably a plurality of such nucleic acid molecules. Typical nucleic acid molecules comprise phosphorous as a component of the nucleic acid backbone, e.g., in the form of a plurality of phosphodiesters or derivatives thereof (e.g., phosphorothioate). The proportion of cation-functionalized chitosan-derivative to nucleic acid can be characterized by a cation (+) to phosphorous (P) molar ratio, wherein the (+) refers to the cation of the cation functionalized chitosan-derivative and the (P) refers to the phosphorous of the nucleic acid backbone. Typically, the (+):(P) molar ratio is selected such that the chitosan-derivative-nucleic acid complex has a positive charge in the absence of PEG-PA polymer molecules. Thus, the (+):(P) molar ratio is generally greater than 1. In preferred embodiments, the (+):(P) molar ratio is greater than 1.5, at least 2, or greater than 2. In certain preferred embodiments, the (+):(P) molar ratio is greater than 2.

In some cases, the (+):(P) molar ratio is, or is about, 3:1. In some cases, the (+): (P) molar ratio is, or is about, 4:1. In some cases, the (+): (P) molar ratio is, or is about, 5:1. In some cases, the (+):(P) molar ratio is, or is about, 6:1. In some cases, the (+):(P) molar ratio is, or is about, 7:1. In some cases, the (+):(P) molar ratio is, or is about, 8:1. In some cases, the (+):(P) molar ratio is, or is about, 9:1. In some cases, the (+):(P) molar ratio is, or is about, 10:1.

In some cases, the (+):(P) molar ratio is from greater than 1 to no more than about 20:1, from about 2 to no more than about 20:1, or from about 2 to no more than about 10:1. In some cases, the (+):(P) molar ratio is from greater than about 2 to no more than about 20:1, or from greater than about 2 to no more than about 10:1. In some cases, the (+):(P) molar ratio is from about 3 to no more than about 20:1, from about 3 to no more than about 10:1, from about 3 to no more than about 8:1, or from about 3 to no more than about 7:1. In some cases, the (+):(P) molar ratio is from about 3 to no more than 20:1, from about 3 to no more than 10:1, from about 3 to no more than 8:1, or from about 3 to no more than 7:1.

In certain embodiments, the (+):(P) molar ratio is 100:1, preferably less than 100:1. For example, in certain embodiments, (+):(P) molar ratio can be from greater than 1 to less than or equal to 100:1. In some cases, the (+):(P) molar ratio can be from greater than 2 to less than or equal to 100:1. In some cases, the (+):(P) molar ratio can be from greater than or equal to 3 to less than or equal to 100:1. In some cases, the (+):(P) molar ratio can be from greater than or equal to 5 to less than or equal to 100:1. In some cases, the (+):(P) molar ratio can be from greater than or equal to 7 to less than or equal to 100:1. In some cases, the (+):(P) molar ratio can be from greater than 2 to less than or equal to 50:1. In some cases, the (+):(P) molar ratio can be from greater than or equal to 3 to less than or equal to 50:1. In some cases, the (+):(P) molar ratio can be from greater than or equal to 5 to less than or equal to 50:1. In some cases, the (+):(P) molar ratio can be from greater than or equal to 7 to less than or equal to 50:1. In some cases, the (+):(P) molar ratio can be from greater than 2 to less than or equal to 25:1. In some cases, the (+):(P) molar ratio can be from greater than or equal to 3 to less than or equal to 25:1. In some cases, the (+):(P) molar ratio can be from greater than or equal to 5 to less than or equal to 25:1. In some cases, the (+):(P) molar ratio can be from greater than or equal to 7 to less than or equal to 25:1.

In some embodiments, the cationic functional group of the chitosan-derivative nanoparticles is or comprises an amino group. Examples of such amino-functionalized chitosan-derivative nanoparticles include, but are not limited to, those containing chitosan that is functionalized with: a guanidinium or a molecule comprising a guanidinium group, a lysine, an ornithine, an arginine, or a combination thereof. In preferred embodiments, the cationic functional group is an arginine. The proportion of amino-functionalized chitosan-derivative to nucleic acid can be characterized by an amino (N) to phosphorous (P) molar ratio, wherein the (N) refers to the nitrogen atom of the amino group in the amino-functionalized chitosan-derivative and the (P) refers to the phosphorous of the nucleic acid backbone. Typically, the N:P molar ratio is selected such that the chitosan-derivative-nucleic acid complex, in the absence of PEG-PA polymer molecules, has a positive charge at a physiologically relevant pH. Thus, the N:P molar ratio is generally greater than 1. In preferred embodiments, the N:P molar ratio is greater than 1.5, at least 2, or greater than 2. In certain preferred embodiments, the N:P molar ration is greater than 2.

In some cases, the N:P molar ratio is, or is about, 3:1. In some cases, the N:P molar ratio is, or is about, 4:1. In some cases, the N:P molar ratio is, or is about, 5:1. In some cases, the N:P molar ratio is, or is about, 6:1. In some cases, the N:P molar ratio is, or is about, 7:1. In some cases, the N:P molar ratio is, or is about, 8:1. In some cases, the N:P molar ratio is, or is about, 9:1. In some cases, the N:P molar ratio is, or is about, 10:1.

In some cases, the N:P molar ratio is from greater than 1 to no more than about 20:1, from about 2 to no more than about 20:1, or from about 2 to no more than about 10:1. In some cases, the N:P molar ratio is from greater than about 2 to no more than about 20:1, or from greater than about 2 to no more than about 10:1. In some cases, the N:P molar ratio is from about 3 to no more than about 20:1, from about 3 to no more than about 10:1, from about 3 to no more than about 8:1, or from about 3 to no more than about 7:1. In some cases, the N:P molar ratio is from about 3 to no more than 20:1, from about 3 to no more than 10:1, from about 3 to no more than 8:1, or from about 3 to no more than 7:1.

In certain embodiments, the N:P molar ratio is 100:1, preferably less than 100:1. For example, in certain embodiments, N:P molar ratio can be from greater than 1 to less than or equal to 100:1. In some cases, the N:P molar ratio can be from greater than 2 to less than or equal to 100:1. In some cases, the N:P molar ratio can be from greater than or equal to 3 to less than or equal to 100:1. In some cases, the N:P molar ratio can be from greater than or equal to 5 to less than or equal to 100:1. In some cases, the N:P molar ratio can be from greater than or equal to 7 to less than or equal to 100:1. In some cases, the N:P molar ratio can be from greater than 2 to less than or equal to 50:1. In some cases, the N:P molar ratio can be from greater than or equal to 3 to less than or equal to 50:1. In some cases, the N:P molar ratio can be from greater than or equal to 5 to less than or equal to 50:1. In some cases, the N:P molar ratio can be from greater than or equal to 7 to less than or equal to 50:1. In some cases, the N:P molar ratio can be from greater than 2 to less than or equal to 25:1. In some cases, the N:P molar ratio can be from greater than or equal to 3 to less than or equal to 25:1. In some cases, the N:P molar ratio can be from greater than or equal to 5 to less than or equal to 25:1. In some cases, the N:P molar ratio can be from greater than or equal to 7 to less than or equal to 25:1.

In a preferred embodiment, the subject polyplexes have amine to phosphate (N/P) ratio of 2 to 100, e.g., 2 to 50, e.g., 2 to 40, e.g., 2 to 30, e.g., 2 to 20, e.g., 2 to 5. Preferably, the N/P ratio is inversely proportional to the molecular weight of the chitosan, i.e., a smaller molecular weight (e.g., dually) derivatized-chitosan requires a higher N/P ratio, and vice versa.

A nucleic acid of the present invention will generally contain phosphodiester bonds, although in some cases nucleic acid analogs are included that may have alternate backbones or other modifications or moieties incorporated for any of a variety of purposes, e.g., stability and protection. Other analog nucleic acids contemplated include those with non-ribose backbones. In addition, mixtures of naturally occurring nucleic acids, analogs, and both can be made. The nucleic acids may be single stranded or double stranded or contain portions of both double stranded or single stranded sequence. Nucleic acids include but are not limited to DNA, RNA and hybrids where the nucleic acid contains any combination of deoxyribo- and ribo-nucleotides, and any combination of bases, including uracil, adenine, thymine, cytosine, guanine, inosine, xanthanine, hypoxanthanine, isocytosine, isoguanine, etc. Nucleic acids include DNA in any form, RNA in any form, including triplex, duplex or single-stranded, anti-sense, siRNA, ribozymes, deoxyribozymes, polynucleotides, oligonucleotides, chimeras, microRNA, and derivatives thereof. Nucleic acids include artificial nucleic acids, including but not limited to, peptide nucleic acid (PNA), phosphorodiamidate morpholino oligo (PMO), locked nucleic acid (LNA), glycol nucleic acid (GNA) and threose nucleic acid (TNA). It will be appreciated that, for artificial nucleic acids that do not comprise phosphorous, an equivalent measure of the (+):P or N:P ratio can be approximated by the number of nucleotide (or nucleotide analog) bases.

In a preferred embodiment, the polyplexes of the compositions comprise chitosan molecules having an average molecular weight of less than 110 kDa, more preferably less than 65 kDa, more preferably less than 50 kDa, more preferably less than 40 kDa, and most preferably less than 30 kDa before functionalization. In some embodiments, polyplexes of the compositions comprise chitosan having an average molecular weight of less than 15 kDa, less than 10 kDa, less than 7 kDa, or less than 5 kDa before functionalization.

In a preferred embodiment, the polyplexes comprise chitosan molecules having on average less than 680 glucosamine monomer units, more preferably less than 400 glucosamine monomer units, more preferably less than 310 glucosamine monomer units, more preferably less than 250 glucosamine monomer units, and most preferably less than 190 glucosamine monomer units. In some embodiments, the polyplexes comprise chitosan molecules having on average less than 95 glucosamine monomer units, less than 65 glucosamine monomer units, less than 45 glucosamine monomer units, or less than 35 glucosamine monomer units.

Chitosan, and (e.g., dually) derivitized-chitosan nucleic acid polyplexes may be prepared by any method known in the art, including but not limited to those described herein.

2.2.1. Nucleic Acids

As described above, the chitosan polyplexes can contain a plurality of nucleic acids. In one embodiment, the nucleic acid component comprises a therapeutic nucleic acid. The subject (e.g., dually) derivatized-chitosan nucleic acid polyplexes are amenable to the use of any therapeutic nucleic acid known in the art. Therapeutic nucleic acids include therapeutic RNAs, which are RNA molecules capable of exerting a therapeutic effect in a mammalian cell. Therapeutic RNAs include, but are not limited to, messenger RNAs, antisense RNAs, siRNAs, short hairpin RNAs, micro RNAs, and enzymatic RNAs. Therapeutic nucleic acids include, but are not limited to, nucleic acids intended to form triplex molecules, protein binding nucleic acids, ribozymes, deoxyribozymes, and small nucleotide molecules.

Many types of therapeutic RNAs are known in the art. For example, see Meng et al., A new developing class of gene delivery: messenger RNA-based therapeutics, Biomater. Sci., 5, 2381-2392, 2017; Grimm et al., Therapeutic application of RNAi is mRNA targeting finally ready for prime time? J. Clin. Invest., 117:3633-3641, 2007; Aagaard et al., RNAi therapeutics: Principles, prospects and challenges, Adv. Drug Deliv. Rev., 59:75-86, 2007; Dorsett et al., siRNAs: Applications in functional genomics and potential as therapeutics, Nat. Rev. Drug Discov., 3:318-329, 2004. These include double-stranded short interfering RNA (siRNA).

Therapeutic nucleic acids also include nucleic acids encoding therapeutic proteins, including cytotoxic proteins and prodrugs.

In a preferred embodiment, the nucleic acid component comprises a therapeutic nucleic acid construct. The therapeutic nucleic acid construct is a nucleic acid construct capable of exerting a therapeutic effect. Therapeutic nucleic acid constructs may comprise nucleic acids encoding therapeutic proteins, as well as nucleic acids that produce transcripts that are therapeutic RNAs. A therapeutic nucleic acid may be used to effect genetic therapy by serving as a replacement or enhancement for a defective gene or to compensate for lack of a particular gene product, by encoding a therapeutic product. A therapeutic nucleic acid may also inhibit expression of an endogenous gene. A therapeutic nucleic acid may encode all or a portion of a translation product, and may function by recombining with DNA already present in a cell, thereby replacing a defective portion of a gene. It may also encode a portion of a protein and exert its effect by virtue of co-suppression of a gene product. In a preferred embodiment, the therapeutic nucleic acid is selected from those disclosed in U.S. 2011/0171314, which is expressly incorporated herein by reference.

In a preferred embodiment, the therapeutic nucleic acid encodes a therapeutic protein that is selected from the group consisting of hormones, enzymes, cytokines, chemokines, antibodies, mitogenic factors, growth factors, differentiation factors, factors influencing angiogenesis, factors influencing blood clot formation, factors influencing blood glucose levels, factors influencing glucose metabolism, factors influencing lipid metabolism, factors influencing blood cholesterol levels, factors influencing blood LDL or HDL levels, factors influencing cell apoptosis, factors influencing food intake, factors influencing energy expenditure, factors influencing appetite, factors influencing nutrient absorption, factors influencing inflammation, and factors influencing bone formation. Particularly preferred are therapeutic nucleic acids encoding insulin, leptin, glucagon antagonist, GLP-1, GLP-2, Ghrelin, cholecystokinin, growth hormone, clotting factors, PYY, erythropoietin, inhibitors of inflammation, IL-10, IL-12, IL-17 antagonists, TNFα antagonists, growth hormone releasing hormone, or parathyroid hormone.

2.2.1.1. Expression Control Regions

In a preferred embodiment, a polyplex of the invention comprises a therapeutic nucleic acid, which is a therapeutic construct, comprising an expression control region operably linked to a coding region. The therapeutic construct produces therapeutic nucleic acid, which may be therapeutic on its own, or may encode a therapeutic protein.

In some embodiments, the expression control region of a therapeutic construct possesses constitutive activity. In a number of preferred embodiments, the expression control region of a therapeutic construct does not have constitutive activity. This provides for the dynamic expression of a therapeutic nucleic acid. By “dynamic” expression is meant expression that changes over time. Dynamic expression may include several such periods of low or absent expression separated by periods of detectable expression. In a number of preferred embodiments, the therapeutic nucleic acid is operably linked to a regulatable promoter. This provides for the regulatable expression of therapeutic nucleic acids.

Expression control regions comprise regulatory polynucleotides (sometimes referred to herein as elements), such as promoters and enhancers, which influence expression of an operably linked therapeutic nucleic acid.

Expression control elements included herein can be from bacteria, yeast, plant, or animal (mammalian or non-mammalian). Expression control regions include full-length promoter sequences, such as native promoter and enhancer elements, as well as subsequences or polynucleotide variants that retain all or part of full-length or non-variant function (e.g., retain some amount of nutrient regulation or cell/tissue-specific expression). As used herein, the term “functional” and grammatical variants thereof, when used in reference to a nucleic acid sequence, subsequence or fragment, means that the sequence has one or more functions of native nucleic acid sequence (e.g., non-variant or unmodified sequence). As used herein, the term “variant” means a sequence substitution, deletion, or addition, or other modification (e.g., chemical derivatives such as modified forms resistant to nucleases).

As used herein, the term “operable linkage” refers to a physical juxtaposition of the components so described as to permit them to function in their intended manner. In the example of an expression control element in operable linkage with a nucleic acid, the relationship is such that the control element modulates expression of the nucleic acid. Typically, an expression control region that modulates transcription is juxtaposed near the 5′ end of the transcribed nucleic acid (i.e., “upstream”). Expression control regions can also be located at the 3′ end of the transcribed sequence (i.e., “downstream”) or within the transcript (e.g., in an intron). Expression control elements can be located at a distance away from the transcribed sequence (e.g., 100 to 500, 500 to 1000, 2000 to 5000, or more nucleotides from the nucleic acid). A specific example of an expression control element is a promoter, which is usually located 5′ of the transcribed sequence. Another example of an expression control element is an enhancer, which can be located 5′ or 3′ of the transcribed sequence, or within the transcribed sequence.

Some expression control regions confer regulatable expression to an operatably linked therapeutic nucleic acid. A signal (sometimes referred to as a stimulus) can increase or decrease expression of a therapeutic nucleic acid operatably linked to such an expression control region. Such expression control regions that increase expression in response to a signal are often referred to as inducible. Such expression control regions that decrease expression in response to a signal are often referred to as repressible. Typically, the amount of increase or decrease conferred by such elements is proportional to the amount of signal present; the greater the amount of signal, the greater the increase or decrease in expression.

Numerous regulatable promoters are known in the art. Preferred inducible expression control regions include those comprising an inducible promoter that is stimulated with a small molecule chemical compound. In one embodiment, an expression control region is responsive to a chemical that is orally deliverable but not normally found in food. Particular examples can be found, for example, in U.S. Pat. Nos. 5,989,910; 5,935,934; 6,015,709; and 6,004,941.

In one embodiment, the therapeutic construct further comprises an integration sequence. In one embodiment, the therapeutic construct comprises a single integration sequence. In another embodiment, the therapeutic construct comprises a first and a second integration sequence for integrating the therapeutic nucleic acid or a portion thereof into the genome of a target cell. In a preferred embodiment, the integration sequence(s) is functional in combination with a means for integration that is selected from the group consisting of mariner, sleeping beauty, FLP, Cre, ΦC31, R, lambda, and means for integration from integrating viruses such as AAV, retroviruses, and lentiviruses.

In one embodiment, the subject composition further comprises a non-therapeutic construct in addition to a therapeutic construct, wherein the non-therapeutic construct comprises a nucleic acid sequence encoding a means for integration operably linked to a second expression control region. This second expression control region and the expression control region operably linked to the therapeutic nucleic acid may be the same or different. The encoded means for integration is preferably selected from the group consisting of mariner, sleeping beauty, FLP, Cre, ΦC31, R, lambda, and means for integration from integrating viruses such as AAV, retroviruses, and lentiviruses.

For further teaching, see WO 2008/020318, which is expressly incorporated herein in its entirety by reference. In one embodiment, the nucleic acid of the (e.g., dually) derivatized-chitosan nucleic acid polyplex is an artificial nucleic acid.

Preferred artificial nucleic acids include, but are not limited to, peptide nucleic acid (PNA), phosphorodiamidate morpholino oligo (PMO), locked nucleic acid (LNA), glycol nucleic acid (GNA) and threose nucleic acid (TNA).

In one embodiment, the nucleic acid of the DD-chitosan nucleic acid polyplex is a therapeutic nucleic acid. In one embodiment, the therapeutic nucleic acid is a therapeutic RNA. Preferred therapeutic RNAs include, but are not limited to, antisense RNA, siRNA, short hairpin RNA, micro RNA, and enzymatic RNA.

In one embodiment, the therapeutic nucleic acid is DNA.

In one embodiment, the therapeutic nucleic acid comprises a nucleic acid sequence encoding a therapeutic protein.

2.3. Polyols

Chitosan-derivative nanoparticles can be functionalized with a polyol. Polyols useful in the present invention in general are typically hydrophilic. In some cases, the chitosan-derivative nanoparticles are functionalized with a cationic component such as an amino group and with a polyol. Such chitosan-derivative nanoparticles functionalized with a cationic moiety such as an amino group and a polyol are referred to as “dually-derivatized chitosan nanoparticles.”

In some embodiments, the chitosan-derivative nanoparticle comprises a polyol of Formula II:

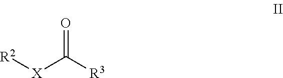

wherein:
R² is selected from: H and hydroxyl;

R³ is selected from: H and hydroxyl; and

X is selected from: C₂-C₆ alkylene optionally substituted with one or more hydroxyl substituents.

In some embodiments, the chitosan-derivative nanoparticle is functionalized with a polyol of Formula II, wherein R² is selected from: H and hydroxyl; R³ is selected from: H and hydroxyl; and X is selected from: C₂-C₆ alkylene optionally substituted with one or more hydroxyl substituents.

In some embodiments, the chitosan-derivative nanoparticle comprises a polyol of Formula III:

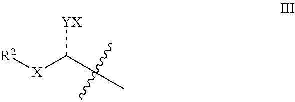

wherein:

### —Y is ═O or —H₂;

R² is selected from: H and hydroxyl;
R³ is selected from: H and hydroxyl;
X is selected from: C₂-C₆ alkylene optionally substituted with one or more hydroxyl substituents; and

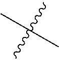

denotes the bond between the polyol and the derivatized chitosan.

In one embodiment, a polyol according to the present invention having 3 to 7 carbons may have one or more carbon-carbon multiple bonds. In a preferred embodiment, a polyol according to the present invention comprises a carboxyl group. In a further preferred embodiment, a polyol according to the present invention comprises an aldehyde group. A skilled artisan will recognize that when a polyol according to the present invention comprises an aldehyde group, such polyol encompasses both the open-chain conformation (aldehyde) and the cyclic conformation (hemiacetal).

Non-limiting examples of a polyols include gluconic acid, threonic acid, glucose and threose. Examples of other such polyols, which may have a carboxyl and/or aldehyde group, or may be a saccharide or acid form thereof, are described in more detail in U.S. Pat. No. 10,046,066, the disclosure of which is expressly incorporated by reference herein. A skilled artisan will recognize that the polyols are not limited to a specific stereochemistry.

In a preferred embodiment, the polyol may be selected from the group consisting of 2,3-dihydroxylpropanoic acid; 2,3,4,5,6,7-hexahydroxylheptanal; 2,3,4,5,6-pentahydroxylhexanal; 2,3,4,5-tetrahydroxylhexanal; and 2,3-dihydroxylpropanal.

In a preferred embodiment, the polyol may be selected from the group consisting of D-glyceric acid, L-glyceric acid, L-glycero-D-mannoheptose, D-glycero-L-mannoheptose, D-glucose, L-glucose, D-fucose, L-fucose, D-glyceraldehyde, and L-glyceraldehyde.

In some embodiments, the polyol may be compound of Formula IV or Formula V:

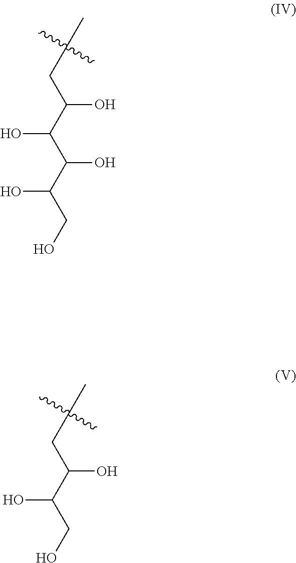 [SMILES: *CC(O)C(O)C(O)C(O)CO ; *CC(O)C(O)CO]

In a preferred embodiment, the polyol is a compound of Formula IV. In some cases, the polyol of Formula IV has been coupled to the chitosan by reductive amination.

A hydrophilic polyol that has a carboxyl group may be coupled to chitosan or a cation functionalized chitosan such as an amine-functionalized chitosan (e.g., Arg-coupled chitosan (Arg-chitosan)). In some embodiments, the polyol is coupled at a reaction pH of 6.0±0.3. At this pH, the carboxylic acid group of the hydrophilic polyol may be attacked by uncoupled amines on the chitosan backbone according to a nucleophilic substitution reaction mechanism.

A hydrophilic polyol that is a natural saccharide may be coupled to chitosan, e.g., cation-functionalized chitosan, such as amine-functionalized chitosan (e.g., Arg-coupled chitosan (Arg-chitosan)) using reductive amination followed by reduction with NaCBH₃ or NaBH.

2.4. Polymer:Polyplex Compositions

Chitosan polyplexes can be mixed with a plurality of polymers, the polymers comprising a hydrophilic, non-charged portion, and a negatively charged (anionic) portion. As described above, the chitosan polyplexes are formulated to have a positive charge in the absence of, or prior to, complexing with the anionic portion-containing polymer. Thus under suitable conditions, the polymer component will form a reversible charge:charge complex with the chitosan-derivative nucleic acid polyplexes. In some embodiments, the polymers of the polymer component are unbranched. In some embodiments, the polymers are branched. In some cases, the polymer component comprises a mixture of branched and unbranched polymers.

In some embodiments, the polymer component is released from the chitosan polyplex after administration, after entering a cell, and/or after endocytosis. Without wishing to be bound by theory, it is hypothesized that the polyplex:polymer compositions thus formed by complexing polyplex and the anionic portion-containing polymer can provide improved in vitro, in solution, and/or in vivo stability without substantially interfering with transfection efficiency. In some embodiments, the polyplex:polymer compositions thus formed can provide reduced muco-adhesive properties as compared to, e.g., otherwise identical, polyplexes without the polymer component.

In a preferred embodiment, the polyplex: polymer compositions have a low net positive, neutral, or net negative zeta potential (from about +10 mV to about −20 mV) at physiological pH. Such compositions can exhibit reduced aggregation in physiological conditions and reduced non-specific binding to ubiquitous anionic components in vivo. Said properties can enhance migration of such composition (e.g., enhanced diffusion in mucus) to contact the cell and result in enhanced intracellular release of nucleic acid.

In a preferred embodiment, the polyplex:polymer particle compositions have an average hydrodynamic diameter of less than 1000 nm, more preferably less than 500 nm and most preferably less than 200 nm. In certain embodiments, the polyplex:polymer particle compositions have an average hydrodynamic diameter of from 50 nm to no more than 1000 nm, preferably from 50 nm to no more than 500 nm and most preferably from 50 nm to no more than 200 nm. In certain embodiments, the polyplex:polymer particle compositions have an average hydrodynamic diameter of from 50 nm to no more than 175 nm, preferably from 50 nm to no more than 150 nm. In certain embodiments, the polyplex:polymer particle compositions have an average hydrodynamic diameter of from 75 nm to no more than 1000 nm, preferably from 75 nm to no more than 500 nm and most preferably from 75 nm to no more than 200 nm. In certain embodiments, the polyplex:polymer particle compositions have an average hydrodynamic diameter of from 75 nm to no more than 175 nm, preferably from 75 nm to no more than 150 nm. In certain embodiments, the polyplex:polymer particle compositions have an average hydrodynamic diameter of greater than 100 nm and less than 175 nm.

In one embodiment, the polyplex:polymer compositions have a % supercoiled DNA content of 80%, at least 80%, or preferably 90%, more preferably at least 90%.

In one embodiment, the polyplex:polymer compositions have an average zeta potential of between +10 mV to −10 mV at a physiological pH, most preferably between +5 mV to −5 mV at a physiological pH.

The polyplex:polymer compositions are preferably homogeneous in respect of particle size. Accordingly, in a preferred embodiment, the composition has a low average polydispersity index (“PDI”). In an especially preferred embodiment, a dispersion of the polyplex:polymer composition has a PDI of less than 0.5, more preferably less than 0.4, more preferably less than 0.3, yet more preferably less than 0.25, and most preferably less than 0.2.

In some cases, a dispersion of the polyplex:polymer composition exhibits one or more of the foregoing PDI, average zeta potential, % supercoil DNA, or average particle size (nm) or size range after one or more freeze thaw cycles. In some cases, a dispersion of the polyplex:polymer composition exhibits one or more of the foregoing PDI, average zeta potential, % supercoil DNA, or average particle size (nm) or size range after storage in solution for at least 48 h at 4° C. In some cases, a dispersion of the polyplex:polymer composition exhibits one or more of the foregoing PDI, average zeta potential, % supercoil DNA, or average particle size (nm) or size range after storage in solution for at least for 2 weeks, or more at 4° C.

In some cases, a dispersion of the polyplex:polymer composition exhibits one or more of the foregoing PDI, average zeta potential, % supercoil DNA, or average particle size (nm) or size range after lyopholization and rehydration. In some cases, a dispersion of the polyplex:polymer composition exhibits one or more of the foregoing PDI, average zeta potential, % supercoil DNA, or average particle size (nm) or size range after spray drying and rehydration. In some cases, a dispersion of the polyplex:polymer composition exhibits one or more of the foregoing PDI, average zeta potential, % supercoil DNA, or average particle size (nm) or size range when concentrated (e.g., by ultrafiltration such as tangential flow filtration) to a nucleic acid concentration of at least 250 μg/mL. In some cases, a dispersion of the polyplex:polymer composition exhibits one or more of the foregoing PDI, average zeta potential, % supercoil DNA, or average particle size (nm) or size range when concentrated to a nucleic acid concentration of from 125 μg/mL to about 1,000 μg/mL. In some cases, a dispersion of the polyplex:polymer composition exhibits one or more of the foregoing PDI, average zeta potential, % supercoil DNA, or average particle size (nm) or size range when concentrated to a nucleic acid concentration of from 125 μg/mL to about 25,000 μg/mL. In some cases, a dispersion of the polyplex:polymer composition exhibits one or more of the foregoing PDI, average zeta potential, % supercoil DNA, or average particle size (nm) or size range when concentrated to a nucleic acid concentration of from 125 μg/mL to about 2,000 μg/mL. In some cases, a dispersion of the polyplex:polymer composition exhibits one or more of the foregoing PDI, average zeta potential, % supercoil DNA, or average particle size (nm) or size range when concentrated to a nucleic acid concentration of from 125 μg/mL to about 5,000 μg/mL. In some cases, a dispersion of the polyplex:polymer composition exhibits one or more of the foregoing PDI, average zeta potential, % supercoil DNA, or average particle size (nm) or size range when concentrated to a nucleic acid concentration of from 125 μg/mL to about 10,000 μg/mL.

In general, the polyplex:polymer compositions described herein, exhibit favorable solution behavior (e.g., stability and/or non-aggregation) as measured by PDI or mean particle size even in the absence of excipients such as lyoprotectants, cryoprotectants, surfactants, rehydration or wetting agents, and the like. In some cases, the polyplex:polymer compositions described herein exhibit favorable solution behavior (e.g., stability and/or non-aggregation) as measured by PDI or mean particle size in physiological fluids or simulated physiological fluids. For example, in some embodiments, the polyplex: polymer compositions described herein are stable in simulated intestinal fluid, instestinal fluid, simulated urine, mammalian urine, when incubated in a mammalian bladder (e.g., and in contact with urine), and/or when incubated in the intestine (e.g., colon, small intestine, or large intestine, preferably the colon). In some embodiments, the polyplex:polymer compositions are stable under one or more of the conditions described herein (e.g., in simulated intestinal fluid) for at least about 10 minutes, or from about 10 minutes to about an hour, or for at least about an hour, or from 1 hour to about 2 hours.

As described above, the polyplex:polymer compositions described herein are preferably substantially size stable in the composition. In a preferred embodiment, a composition of the invention comprises polyplex:polymer particles that increase in average diameter by less than 100%, more preferably less than 50%, and most preferably less than 25%, at room temperature for 6 hours, more preferably 12 hours, more preferably 24 hours, and most preferably 48 hours. In a particularly preferred embodiment, a composition of the invention comprises polyplex:polymer particles that increase in average diameter by less than 25% at room temperature for at least 24 hours or at least 48 hours.

The polyplex:polymer particles of the subject compositions are preferably substantially size stable under cooled conditions. In a preferred embodiment, a composition of the invention comprises polyplex:polymer particles that increase in average diameter by less than 100%, more preferably less than 50%, and most preferably less than 25%, at 2-8 degrees Celsius for 6 hours, more preferably 12 hours, more preferably 24 hours, and most preferably 48 hours.

The polyplex:polymer particles of the subject compositions are preferably substantially size stable under freeze-thaw conditions. In a preferred embodiment, a composition of the invention comprises polyplexes that increase in average diameter by less than 100%, more preferably less than 50%, and most preferably less than 25% at room temperature for 6 hours, more preferably 12 hours, more preferably 24 hours, and most preferably 48 hours following thaw from frozen at −20 to −80 degrees Celsius.

In a preferred embodiment, the composition has a nucleic acid concentration greater than 0.5 mg/ml, and is substantially free of precipitated polyplex. More preferably, the composition has a nucleic acid concentration of at least 0.6 mg/ml, more preferably at least 0.75 mg/ml, more preferably at least 1.0 mg/ml, more preferably at least 1.2 mg/ml, and most preferably at least 1.5 mg/ml, and is substantially free of precipitated polyplex. In another preferred embodiment, the composition has a nucleic acid concentration greater than 2 mg/ml, and is substantially free of precipitated polyplex. More preferably, the composition has a nucleic acid concentration of at least 2.5 mg/ml, more preferably at least 5 mg/ml, more preferably at least 10 mg/ml, more preferably at least 15 mg/ml, and most preferably about 25 mg/ml, and is substantially free of precipitated polyplex. In some embodiments, the composition has a nucleic acid concentration from 0.5 mg/mL to about 25 mg/mL, and is substantially free of precipitated polyplex. In some embodiments, the composition has a nucleic acid concentration of ≤about 25 mg/mL, and is substantially free of precipitated polyplex. The compositions can be hydrated. In a preferred embodiment, the composition is substantially free of uncomplexed nucleic acid.

In a preferred embodiment, the polyplex:polymer particle composition is isotonic. Achieving isotonicity, while maintaining polyplex stability, is highly desirable in formulating pharmaceutical compositions, and these preferred compositions are well suited to pharmaceutical formulation and therapeutic applications.

In certain embodiments, the polyplex:polymer particle composition can be uncoated to release all or part of the, e.g., PEG, polymer coat by reducing pH. In certain embodiments, the polymer coat is released by incubating the particle under a pH condition that is below the pKa of the polyanionic anchor region of the polymer. For example, where the polymer coat is polyglutamate, the polymer coat can be released by incubating the particle at a pH below the pKa of polyglutamate, such as a pH of less than about 4.25. In certain embodiments, the polymer coat can be released by incubating the particle under a pH condition that is at least 0.25 pH units or at least 0.5 pH units below the pKa of the polyanion anchor region of the polymer coat.

In certain embodiments, the polyplex:polymer particle composition can be uncoated to release all or part of the, e.g., PEG, polymer coat by subjecting the particle to a high ionic strength.

Without wishing to be bound by theory, it is hypothesized that certain physiological conditions can promote partial (e.g., >5%), substantial (>50%), extensive (e.g., >90%), or total uncoating of reversibly PEGylated chitosan DNA polyplexes described herein. For example, low pH conditions in certain subcellular compartments (e.g., endosome, early endosome, late endosome, or lysosome) can facilitate release of the polymer coat. As another example, certain extracelluar conditions can promote partial (e.g. >5%), substantial (>50%), extensive (e.g. >90%), or complete (100%) uncoating of reversibly PEGylated chitosan DNA polyplexes described herein. In some cases, the high ionic strength and/or acidic pH conditions typically encounted in certain positions in the alimentary canal can promote partial (e.g. >5%), substantial (>50%), extensive (e.g. >90%), or complete (100%) uncoating of reversibly PEGylated chitosan DNA polyplexes described herein.

In certain embodiments, PEGylated polyplexes described herein are formulated for delivery to a cell, tissue, or bodily compartment (e.g., intestine, small intestine, large intestine, colon, lung, or bladder) such that the polyplexes remain PEGylated and thereby facilitate transfection of a target cell. In some embodiments, PEGylated polyplexes described herein partially (e.g. >5%), substantially (>50%), extensively (e.g., >90%), or completely (100%) release the polymer coat after or during entry into the intracellular environment. In certain embodiments, PEGylated polyplexes described herein are formulated for delivery to a cell, tissue, or bodily compartment (e.g., intestine, small intestine, large intestine, colon, lung, or bladder) such that the PEGylated polyplexes described herein partially (e.g. >5%), substantially (>50%), extensively (e.g. >90%), or completely (100%) release the polymer coat upon delivery to a cell, tissue, or bodily compartment (e.g., intestine, small intestine, large intestine, colon, lung, or bladder).

It will be appreciated that anion charge density and/or pKa of the anionic anchor region of a polymer can be adjusted to promote or inhibit release under intended conditions. It will similarly be appreciated that the pH, volume, and ionic strength, and other conditions of the formulation can be adjusted to promote or inhibit release under intended conditions. For example, for delivery to the intestine through the low pH gastric environment, a PEGyalted polyplex formulation can be enteric coated and/or delivered in a buffering agent to increase the pH of the gastric environment. Optimized reversibly PEGylated particle compositions and formulations can be identified by assaying for stability and transfection efficiency using assays described herein.

The compositions comprising chitosan polyplex complexed with the anionic portion-containing polymer can be characterized by the ratio of cationic functional groups of the (e.g., dually) derivatized-chitosan polyplex (+) to anion moieties of the polymer (−), referred to as the “(+):(−) molar ratio”. This (+):(−) molar ratio can vary from greater than about 1:100 to less than about 10:1.

In certain embodiments, the (+):(−) molar ratio can be from greater than about 1:75 to less than about 8:1. In some cases, the (+):(−) molar ratio can be from greater than 1:10 to less than 10:1. In some cases, the (+):(−) molar ratio can be from, or from about, 1:10 to, or to about, 10:1. In some cases, the (+):(−) molar ratio can be from, or from about, 1:8 to, or to about, 8:1. In certain embodiments, the (+):(−) molar ratio can be from greater than 1:50 to less than about 10:1. In some cases, the (+):(−) molar ratio can be from greater than 1:25 to less than about 10:1. In some cases, the (+):(−) molar ratio can be from greater than 1:10 to less than about 7:1. In some cases, the (+):(−) molar ratio can be from greater than 1:8 to less than about 7:1. In some cases, the (+):(−) molar ratio can be from greater than 1:8 to less than about 6:1.

In certain embodiments, where the cationic functional group of the (e.g., dually) derivatized-chitosan polyplex is an amino moiety, the compositions comprising chitosan polyplex complexed with the anionic portion-containing polymer can be characterized by the ratio of amino groups of the (e.g., dually) derivatized-chitosan polyplex (N) to anion (A) moieties of the polymer, referred to as the “N:A molar ratio”. This N:A molar ratio can vary from greater than about 1:100 to less than about 10:1.

In certain embodiments, the N: A molar ratio can be from greater than about 1:75 to less than about 8:1. In some cases, the N:A molar ratio can be from greater than 1:10 to less than 10:1. In some cases, the N:A molar ratio can be from, or from about, 1:10 to, or to about, 10:1. In some cases, the N:A molar ratio can be from, or from about, 1:8 to, or to about, 8:1. In certain embodiments, the N:A molar ratio can be from greater than 1:50 to less than about 10:1. In some cases, the N:A molar ratio can be from greater than 1:25 to less than about 10:1. In some cases, the N:A molar ratio can be from greater than 1:10 to less than about 7:1. In some cases, the N:A molar ratio can be from greater than 1:8 to less than about 7:1. In some cases, the N:A molar ratio can be from greater than 1:8 to less than about 6:1.

Additionally or alternatively, the compositions comprising chitosan polyplex complexed with the anionic portion-containing polymer can be characterized by a three-component ratio of cationic functional groups of the (e.g., dually) derivatized-chitosan polyplex (+) to phosphorus atoms of the nucleic acid (P) to anion moieties of the polymer (−), referred to as the “(+):P:(−) molar ratio”.

In certain embodiments, where (+):P is from at least 2:1 to no more than 20:1, the molar ratio of (+):(−) can vary from at least 1:40 to about 40:1. In certain embodiments, where (+):P is from at least 2:1 to no more than 20:1, the molar ratio of (+):(−) can vary from at least 1:40 to about 1:10. In some embodiments, where (+):P is from at least 2:1 to no more than 20:1, the molar ratio of (+):(−) can vary from at least 1:25 to about 25:1. In some embodiments, where (+):P is from at least 2:1 to no more than 20:1, the molar ratio of (+):(−) can vary from at least 1:25 to about 1:10. In some cases, where (+):P is from at least 2:1 to no more than 20:1, the molar ratio of (+):(−) can vary from at least 1:20 to about 20:1. In some cases, where (+):P is from at least 2:1 to no more than 20:1, the molar ratio of (+):(−) can vary from at least 1:20 to about 1:10. In some cases, where (+):P is from at least 2:1 to no more than 20:1, the molar ratio of (+):(−) can vary from at least 1:10 to about 10:1. In some cases, where (+):P is from at least 2:1 to no more than 20:1, the molar ratio of (+):(−) can vary from at least 1:25 to about 2:1. In some cases, where (+):P is from at least 2:1 to no more than 20:1, the molar ratio of (+):(−) can vary from at least 1:20 to about 1:1.

In certain preferred embodiments, (+):P:(−) is from 3:1:3.5 to 3:1:17.5. In certain preferred embodiments, (+):P:(−) is from 5:1:3.5 to 5:1:17.5. In certain preferred embodiments, (+):P:(−) is from 7:1:3.5 to 7:1:17.5. In certain preferred embodiments, (+):P:(−) is about 3:1:3.5, 3:1:7, 3:1:10, 3:1:15, 3:1:17.5, or 3:1:20. In certain preferred embodiments, (+):P:(−) is about 5:1:3.5, 5:1:7, 5:1:10, 5:1:15, 5:1:17.5, or 5:1:20. In certain preferred embodiments, (+):P:(−) is about 7:1:3.5, 7:1:7, 7:1:10, 7:1:15, 7:1:17.5, or 7:1:20. In certain preferred embodiments, (+):P:(−) is about 10:1:10, 10:1:15, 10:1:20, 10:1:25, 10:1:30, or 10:1:40.

One of skill in the art will appreciate that amino-functionalized chitosan polyplex particles in complex with the anionic portion-containing polymer can be characterized by a three-component ratio of amino functional groups of the (e.g., dually) derivatized-chitosan polyplex (N) to phosphourus atoms of the nucleic acid (P) to anion moieties of the polymer (A), referred to as the “N:P:A molar ratio”. In certain embodiments, where N:P is from at least 2:1 to no more than 20:1, the molar ratio of P:A can vary from at least 1:40 to about 40:1.

In certain embodiments, where N:P is from at least 2:1 to no more than 20:1, the molar ratio of P:A can vary from at least 1:40 to about 1:10. In certain embodiments, where N:P is from at least 2:1 to no more than 20:1, the molar ratio of P:A can vary from at least 1:25 to about 25:1. In certain embodiments, where N:P is from at least 2:1 to no more than 20:1, the molar ratio of P:A can vary from at least 1:25 to about 1:10. In some cases, where N:P is from at least 2:1 to no more than 20:1, the molar ratio of P:A can vary from at least 1:20 to about 20:1. In some cases, where N:P is from at least 2:1 to no more than 20:1, the molar ratio of P:A can vary from at least 1:20 to about 1:10. In some cases, where N:P is from at least 2:1 to no more than 20:1, the molar ratio of P:A can vary from at least 1:10 to about 10:1. In some cases, where N:P is from at least 2:1 to no more than 20:1, the molar ratio of P:A can vary from at least 1:25 to about 2:1. In some cases, where N:P is from at least 2:1 to no more than 20:1, the molar ratio of P:A can vary from at least 1:20 to about 1:1.

In certain preferred embodiments, N:P:A is from 3:1:3.5 to 3:1:17.5. In certain preferred embodiments, N:P:A is from 5:1:3.5 to 5:1:17.5. In certain preferred embodiments, N:P:A is from 7:1:3.5 to 7:1:17.5. In certain preferred embodiments, N:P:A is from 10:1:10 to 10:1:40. In certain preferred embodiments, N:P:A is about 3:1:3.5, 3:1:7, 3:1:10, 3:1:15, 3:1:17.5, or 3:1:20. In certain preferred embodiments, N:P:A is about 5:1:3.5, 5:1:7, 5:1:10, 5:1:15, 5:1:17.5, or 5:1:20. In certain preferred embodiments, N:P:A is about 7:1:3.5, 7:1:7, 7:1:10, 7:1:15, 7:1:17.5, or 7:1:20. In certain embodiment, N:P:A is about 10:1:10, 10:1:15, 10:1:20, 10:1:25, 10:1:30 or 10:1:40.

2.4.1. Hydrophilic Non-Charged Portion

The hydrophilic non-charged portion of the polymer can be, or comprise, a polyalkylene polyol or a polyalkyleneoxy polyol portion, or combinations thereof. The hydrophilic non-charged portion of the polymer can be, or comprise, a polyalkylene glycol or polyalkyleneoxy glycol portion. In certain embodiments, the polyalkylene glycol portion is or comprises a polyethylene glycol portion and/or a monomethoxy polyethylene glycol portion. In certain preferred embodiments, the non-charged portion of the polymer is, or comprises polyethylene glycol. The hydrophilic non-charged portion of the polymer can be, or comprise, other biologically compatible polymer(s) such as polylactic acid.

In addition to PEG, several hydrophilic non-charged entities are known in the art. For example, see: Lowe et. al., Antibiofouling polymer interfaces: poly(ethyleneglycol) and other promising candidates, Polym. Chem., 6, 198-212, 2015, and Knop et. al., Poly(ethylene glycol) in Drug Delivery: Pros and Cons as Well as Potential Alternatives. Angewandte Chemie International Edition, 49(36), 6288-6308, 2010. Examples of hydrophilic non-charged portion of the polymer are but not limited to: poly(glycerol), poly(2-methacryloyloxyethyl phosphorylcholine), poly(sulfobetaine methacrylate), and poly(carboxybetaine methacrylate), poly(2-methyl-2-oxazoline), poly(2-ethyl-2-oxazoline), and poly(vinylpyrrolidone)

The hydrophilic portion can have a weight average molecular weight of from about 500 Da to about 50,000 Da. In some embodiments, the hydrophilic portion has a weight average molecular weight of from about 1,000 Da to about 10,000 Da. In certain embodiments, the hydrophilic portion has a weight average molecular weight of from about 1,500 Da to about 7,500 Da. In certain embodiments, the hydrophilic portion has a weight average molecular weight of from about 3,000 Da to about 5,000 Da. In some cases, the hydrophilic portion has a weight average molecular weight of, or of about, 5,000 Da.

2.4.2. Anionic Polymer Portion

The anionic polymer portion of the polymer can comprise a plurality of functional groups that are negatively charged at physiological pH. A wide variety of anionic polymers are suitable for use in the methods and compositions described herein, provided that such anionic polymers can be provided as a component of a polymer having a hydrophilic non-charged polymer portion and are capable of forming a (e.g., reversible) charge:charge complex with the positively charged (e.g., dually) derivatized-chitosan-nucleic acid nanoparticles.

Exemplary anionic polymers include, but are not limited to, polypeptides having a net negative charge at physiological pH. In some cases, the polypeptides, or a portion thereof, consist of amino acids having a negatively charged side-chain at physiological pH. For example, the anionic polymer portion of the polymer can be a polyglutamate polypeptide, a polyaspartate polypeptide, or a mixture thereof. Additional amino acids, or mimetics thereof, can be incorporated into the polyanionic polypeptide. For example, glycine and/or serine amino acids can be incorporated to increase flexibility or reduce secondary structure.

In some cases, the anionic polymers can be or comprise an anionic carbohydrate polymer. Exemplary anionic carbohydrate polymers include, but are not limited to, glycosaminoglycans that are negatively charged at physiological pH. Exemplary anionic glycosaminoglycans include, but are not limited to, chondroitin sulfate, dermatan sulfate, keratin sulfate, heparin, heparin sulfate, hyaluronic acid, or a combination thereof. In certain embodiments, the anionic polymer portion of the polymer is or comprises hyaluronic acid.

Additional or alternative anionic carbohydrate polymers can include polymers comprising dextran sulfate.

In some cases, the polyanion portion is, or comprises, a polyanion selected from the group consisting of polymethacrylic acid and its salts, polyacrylic acid and its salts, copolymers of methacrylic acids and its salts, and copolymers of acrylic acid and/or methacrylic acid and its salts, such as a polyalkylene oxide, polyacrylic acid copolymer.

In some cases, the polyanion portion is, or comprises, a polyanion is selected from the group consisting of alginate, carrageenan, furcellaran, pectin, xanthan, hyaluronic acid, heparin, heparan sulfate, chondroitin sulfate, cellulose, oxidized cellulose, carboxymethyl celluose, crosmarmelose, syntheic polymers and copolymers containing pendant carboxyl groups, phosphate groups or sulphate groups, polyaminoacids of predominantly negative charge, and biocompatible polyphenolic materials.

The anionic portion of the polymers can have a weight average molecular weight of from about 500 Da to about 5,000 Da. In some embodiments, the anionic portion has a weight average molecular weight of from about 500 Da to about 3,000 Da. In certain embodiments, the anionic portion has a weight average molecular weight of from about 500 Da to about 2,500 Da. In certain embodiments, the anionic portion has a weight average molecular weight of from about 500 Da to about 2,000 Da. In certain embodiments, the anionic portion has a weight average molecular weight of from about 500 Da to about 1,500 Da. In some embodiments, the anionic portion has a weight average molecular weight of from about 1,000 Da to about 5,000 Da. In some embodiments, the anionic portion has a weight average molecular weight of from about 1,000 Da to about 3,000 Da. In certain embodiments, the anionic portion has a weight average molecular weight of from about 1,000 Da to about 2,500 Da. In certain embodiments, the anionic portion has a weight average molecular weight of from about 1,000 Da to about 2,000 Da. In some cases, the aninoic portion has a weight average molecular weight of, or of about, 1,500 Da.

As used herein, “block copolymer”, “block co-polymer”, and the like refers to a copolymer containing distinct homopolymer regions. A diblock copolymer contains two distinct homopolymer regions. A triblock copolymer contains three distinct homopolymer regions. The three distinct regions can each be different (e.g., AAAA-BBBB-CCCC), or two regions can be the same (e.g., AAAA-BBBB-AAAA) similar (e.g., AAAA-BBBB-AAA), wherein “A”, “B”, and “C” represent different monomer subunits that form copolymer is comprised. For example, “A” can represent an ethylene glycol monomer subunit of a polyethylene glycol homopolymer and B can represent a glutamic acid subunit of a polyglutamic acid homopolymer. The block copolymer can be a linear (e.g., di- or tri-) block copolymer. Exemplary embodiments of linear diblock and triblock copolymers for use in the subject invention include those listed in the following non-exhaustive list:

PEG-Polyglutamic acid
methoxy-poly(ethylene glycol)-block-poly(L-glutamic acid)
mPEG*K-b-PLE##
mPEG1K-b-PLE10
mPEG1K-b-PLE50
mPEG1K-b-PLE100
mPEG1K-b-PLE200
mPEG5K-b-PLE10
mPEG5K-b-PLE50
mPEG5K-b-PLE100
mPEG5K-b-PLE200
mPEG10K-b-PLE10
mPEG10K-b-PLE50
mPEG10K-b-PLE100
mPEG10K-b-PLE200
mPEG20K-b-PLE10
mPEG20K-b-PLE50
mPEG20K-b-PLE100
mPEG20K-b-PLE200
PEG-Polyaspartic acid
methoxy-poly( ethylene glycol)-block-poly(L-aspartic acid)
mPEG*K-b-PLD##
mPEG1K-b-PLD10
mPEG1K-b-PLD50
mPEG1K-b-PLD100
mPEG1K-b-PLD200
mPEG5K-b-PLD10
mPEG5K-b-PLD50
mPEG5K-b-PLD100
mPEG5K-b-PLD200
mPEG20K-b-PLD10
mPEG20K-b-PLD50
mPEG20K-b-PLD100
mPEG20K-b-PLD200
PGA-PEG-PGA
poly(L-glutamic acid)-block-poly(ethylene glycol)-block-
poly(L-glutamic acid)
PLE##-b-PEG*K-b-PLE##
PLE10-b-PEG1K-b-PLE10
PLE50-b-PEG1K-b-PLE50
PLE100-b-PEG1K-b-PLE100
PLE10-b-PEG5K-b-PLE10
PLE50-b-PEG5K-b-PLE50
PLE100-b-PEG5K-b-PLE100
Polyaspartic-PEG-polyaspartic
poly(L-aspartic acid)-block-poly(ethylene glycol)-block-
poly(L-aspartic acid)
PLD##-b-PEG*K-b-PLD##
PLD10-b-PEG1K-b-PLD10
PLD50-b-PEG1K-b-PLD50
PLD100-b-PEG1K-b-PLD100
PLD10-b-PEG5K-b-PLD10
PLD50-b-PEG5K-b-PLD50
PLD100-b-PEG5K-b-PLD100
PEG- poly glutamic acid -PEG
Methoxy-poly(ethylene glycol)-block-poly(L-glutamic acid)-block-
poly(ethylene glycol)
PEG*K-b-PGA##-b-PEG*K
PEG1K-b-PGA10-b-PEG1K
PEG1K-b-PGA50-b-PEG1K
PEG1K-b-PGA100-b-PEG1K
PEG5K-b-PGA10-b-PEG5K
PEG5K-b-PGA50-b-PEG5K
PEG5K-b-PGA100-b-PEG5K
PEG- polyaspartic-PEG
Methoxy-poly(ethylene glycol)-block-poly(L-aspartic acid)-block-
poly(ethylene glycol)
PEG*K-b-PLD##-b-PEG*K
PEG1K-b-PLD10-b-PEG1K
PEG1K-b-PLD50-b-PEG1K
PEG1K-b-PLD100-b-PEG1K
PEG5K-b-PLD10-b-PEG5K
PEG5K-b-PLD50-b-PEG5K
PEG5K-b-PLD100-b-PEG5K
*K: molecular weight of PEG in kDa
## number of subunits

In one embodiment, the block copolymer is or comprises a PEG-polyglutamic acid polymer having the following structure:

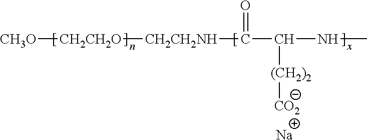

In one embodiment, the block copolymer is or comprises a PEG-polyaspartic acid polymer having the following structure:

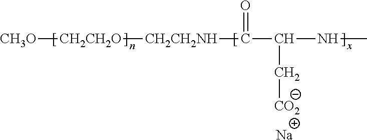

In one embodiment, the block copolymer is or comprises a PEG-hyaluronic acid polymer having the following structure:

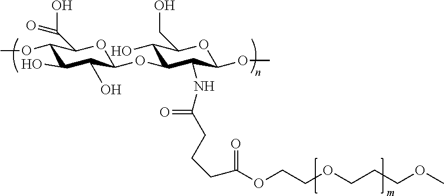

2.5. Methods of Making

As described above, one of skill in the art will appreciate that polyplex:polymer particles of the invention may be produced by a variety of methods. For example, polyplex particles can be generated and then contacted with polymer. In an exemplary non-limiting embodiment, polyplex particles are prepared by providing and combining functionalized chitosan and nucleotide feedstock. Feedstock concentrations may be adjusted to accommodate various amino-to-phosphate ratios (N/P), mixing ratios and target nucleotide concentrations. In some embodiments, particularly small batches, e.g., batches under 2 mL, the functionalized chitosan and nucleotide feedstocks may be mixed by slowly dripping the nucleotide feedstock into the functionalized chitosan feedstock while vortexing the container. In other embodiments, the functionalized chitosan and nucleotide feedstocks may be mixed by in-line mixing the two fluid streams. In other embodiments, the resulting polyplex dispersion may be concentrated by means known in the art such as ultrafiltration (e.g., tangential flow filtration (TFF)), or solvent evaporation (e.g., lyopholization or spray drying). A preferred method for polyplex formation is disclosed in WO 2009/039657, which is expressly incorporated herein in its entirety by reference.

Similarly, polyplex particle feedstock (e.g., an aqueous solution comprising the polyplex compositions) can be provided (e.g., isolated from the reaction mixtures described above) and combined with polymer feedstock (e.g., an aqueous solution comprising the polymer). Feedstock concentrations may be adjusted to accommodate various amino-to-anion ratios (N/A), amino-to-phosphorous (N:P) ratios, N:P:A ratios, mixing ratios and target nucleotide concentrations. In some embodiments, particularly small batches, e.g., batches under 2 mL, the feedstocks may be mixed by slowly dripping a first feedstock (e.g., polyplex) into a second feedstock (e.g., polymer) while vortexing the container. In other embodiments, the feedstocks may be mixed by in-line mixing the two fluid streams. In other embodiments, the resulting polyplex:polymer complex dispersion may be concentrated by means known in the art such as ultrafiltration (e.g., tangential flow filtration (TFF)), or solvent evaporation (e.g., lyopholization or spray drying).

In some embodiments, the polyplex:polymer composition comprises a core-shell type particle composition, wherein the particles comprise a polyplex core and a non-covalently bound (e.g., releasable) polymer shell. As will be appreciated, one method for making such a core-shell type composition includes forming the polyplex and then combining with polymer feedstock as described above.

Alternatively, the polyplex:polymer composition can be made in a one-step method in which nucleic acid, derivatized chitosan, and a plurality of linear block copolymers comprising at least one polyanionic (PA) anchor region and at least one hydrophilic polymer (e.g., PEG) tail region are mixed at appropriate ratios to form a polyplex:polymer composition. Without wishing to be bound by theory, the present inventors hypothesize that such one-step method can produce smaller particle sizes that may be advantageous in certain indications where in vivo mucosal diffusion is limited.

### 3. Powdered Formulations

The polyplex:polymer compositions of the invention include powders. In a preferred embodiment, the invention provides a dry powder polyplex:polymer composition. In a preferred embodiment, the dry powder polyplex:polymer composition is produced through the dehydration (e.g., spray drying or lyopholization) of a chitosan-nucleic acid polyplex dispersion of the invention.

### 4. Pharmaceutical Formulations

The present invention also provides “pharmaceutically acceptable” or “physiologically acceptable” formulations comprising polyplex:polymer compositions of the invention. Such formulations can be administered in vivo to a subject in order to practice treatment methods.

As used herein, the terms “pharmaceutically acceptable” and “physiologically acceptable” refer to carriers, diluents, excipients and the like that can be administered to a subject, preferably without producing excessive adverse side-effects (e.g., nausea, abdominal pain, headaches, etc.). Such preparations for administration include sterile aqueous or non-aqueous solutions, suspensions, and emulsions. Liquid formulations include suspensions, solutions, syrups and elixirs. Liquid formulations may be prepared by the reconstitution of a solid.

Pharmaceutical formulations can be made from carriers, diluents, excipients, solvents, dispersion media, coatings, antibacterial and antifungal agents, isotonic and absorption delaying agents, and the like, compatible with administration to a subject. Such formulations can be contained in a tablet (coated or uncoated), capsule (hard or soft), microbead, emulsion, powder, granule, crystal, suspension, syrup or elixir. Supplementary active compounds and preservatives, among other additives, may also be present, for example, antimicrobials, anti-oxidants, chelating agents, and inert gases and the like.

Excipients can include a salt, an isotonic agent, a serum protein, a buffer or other pH-controlling agent, an anti-oxidant, a thickener, an uncharged polymer, a preservative or a cryoprotectant. Excipients used in compositions of the invention may further include an isotonic agent and a buffer or other pH-controlling agent. These excipients may be added for the attainment of preferred ranges of pH (about 6.0-8.0) and osmolarity (about 50-400 mmol/L). Examples of suitable buffers are acetate, borate, carbonate, citrate, phosphate and sulfonated organic molecule buffer. Such buffers may be present in a composition in concentrations from 0.01 to 1.0% (w/v). An isotonic agent may be selected from any of those known in the art, e.g. mannitol, dextrose, glucose and sodium chloride, or other electrolytes. Preferably, the isotonic agent is glucose or sodium chloride. The isotonic agents may be used in amounts that impart to the composition the same or a similar osmotic pressure as that of the biological environment into which it is introduced. The concentration of isotonic agent in the composition will depend upon the nature of the particular isotonic agent used and may range from about 0.1 to 10%. When glucose is used, it is preferably used in a concentration of from 1 to 5% w/v, more particularly 5% w/v. When the isotonic agent is sodium chloride, it is preferably employed in amounts of up to 1% w/v, in particular 0.9% w/v. The compositions of the invention may further contain a preservative. Examples preservatives are polyhexamethylene-biguanidine, benzalkonium chloride, stabilized oxychloro complexes (such as those known as Purite®), phenylmercuric acetate, chlorobutanol, sorbic acid, chlorhexidine, benzyl alcohol, parabens, and thimerosal. Typically, such preservatives are present at concentrations from about 0.001 to 1.0%. Furthermore, the compositions of the invention may also contain a cryopreservative agent. Preferred cryopreservatives are glucose, sucrose, mannitol, lactose, trehalose, sorbitol, colloidal silicon dioxide, dextran of molecular weight preferable below 100,000 g/mol, glycerol, and polyethylene glycols of molecular weights below 100,000 g/mol or mixtures thereof. Most preferred are glucose, trehalose and polyethylene glycol. Typically, such cryopreservatives are present at concentrations from about 0.01 to 10%.

A pharmaceutical formulation can be formulated to be compatible with its intended route of administration. For example, for oral administration, a composition can be incorporated with excipients and used in the form of tablets, troches, capsules, e.g., gelatin capsules, or coatings, e.g., enteric coatings (Eudragit® or Sureteric®). Pharmaceutically compatible binding agents, and/or adjuvant materials can be included in oral formulations. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or other stearates; a glidant such as colloidal silicon dioxide; a sweetening agent such as sucrose or saccharin; or a flavoring agent such as peppermint, methyl salicylate, or flavoring.

Formulations can also include carriers to protect the composition against rapid degradation or elimination from the body, such as a controlled release formulation, including implants and microencapsulated delivery systems. For example, a time delay material such as glyceryl monostearate or glyceryl stearate alone, or in combination with a wax, may be employed.

Suppositories and other rectally administrable formulations (e.g., those administrable by enema) are also contemplated. Further regarding rectal delivery, see, for example, Song et al., Mucosal drug delivery: membranes, methodologies, and applications, Crit. Rev. Ther. Drug. Carrier Syst., 21:195-256, 2004; Wearley, Recent progress in protein and peptide delivery by noninvasive routes, Crit. Rev. Ther. Drug. Carrier Syst., 8:331-394, 1991.

Additional pharmaceutical formulations appropriate for administration are known in the art and are applicable in the methods and compositions of the invention (see, e.g., Remington's Pharmaceutical Sciences (1990) 18th ed., Mack Publishing Co., Easton, Pa.; The Merck Index (1996) 12th ed., Merck Publishing Group, Whitehouse, N.J.; and Pharmaceutical Principles of Solid Dosage Forms, Technonic Publishing Co., Inc., Lancaster, Pa., (1993)).

### 5. Administration

In one embodiment, the use of polyplexes:polymer compositions provides for prolonged stability of polyplexes at physiological pH. This provides for effective mucosal administration.

Any of a number of administration routes to contact mucosal cells or tissue are possible and the choice of a particular route will in part depend on the target mucosal cell or tissue. Syringes, endoscopes, cannulas, intubation tubes, catheters and other articles may be used for administration.

The doses or “effective amount” for treating a subject are preferably sufficient to ameliorate one, several or all of the symptoms of the condition, to a measurable or detectable extent, although preventing or inhibiting a progression or worsening of the disorder or condition, or a symptom, is a satisfactory outcome. Thus, in the case of a condition or disorder treatable by expressing a therapeutic nucleic acid in target tissue, the amount of therapeutic RNA or therapeutic protein produced to ameliorate a condition treatable by a method of the invention will depend on the condition and the desired outcome and can be readily ascertained by the skilled artisan. Appropriate amounts will depend upon the condition treated, the therapeutic effect desired, as well as the individual subject (e.g., the bioavailability within the subject, gender, age, etc.). The effective amount can be ascertained by measuring relevant physiological effects.

Veterinary applications are also contemplated by the present invention. Accordingly, in one embodiment, the invention provides methods of treating non-human mammals, which involve administering a polyplex:polymer composition of the invention to a non-human mammal in need of treatment.

5.1. Oral Administration

The subject compositions may be administered orally. Oral administration may involve swallowing, so that the compound enters the gastrointestinal tract. Compositions of the invention may also be administered directly to the gastrointestinal tract.

Formulations suitable for oral administration include solid formulations such as tablets, capsules, coated capsules containing particulates or coated particulates, liquids, or powders, lozenges (including liquid-filled), chews, multi- and nano-particulates, gels, films, ovules, and sprays.

Tablet dosage forms generally contain a disintegrant. Examples of disintegrants include sodium starch glycolate, sodium carboxymethyl cellulose, calcium carboxymethyl cellulose, croscarmellose sodium, crospovidone, polyvinylpyrrolidone, methyl cellulose, microcrystalline cellulose, lower alkyl-substituted hydroxypropyl cellulose, starch, pregelatinised starch and sodium alginate. Generally, the disintegrant will comprise from 1 weight % to 25 weight %, preferably from 5 weight % to 20 weight % of the dosage form

Binders are generally used to impart cohesive qualities to a tablet formulation. Suitable binders include microcrystalline cellulose, gelatin, sugars, polyethylene glycol, natural and synthetic gums, polyvinylpyrrolidone, pregelatinised starch, hydroxypropyl cellulose and hydroxypropyl methylcellulose. Tablets may also contain diluents, such as lactose (monohydrate, spray-dried monohydrate, anhydrous and the like), mannitol, xylitol, dextrose, sucrose, sorbitol, microcrystalline cellulose, starch and dibasic calcium phosphate dihydrate.

Tablets may also optionally comprise surface active agents, such as sodium lauryl sulfate and polysorbate 80, and glidants such as silicon dioxide and talc. When present, surface active agents may comprise from 0.2 weight % to 5 weight % of the tablet, and glidants may comprise from 0.2 weight % to 1 weight % of the tablet.

Tablets also generally contain lubricants such as magnesium stearate, calcium stearate, zinc stearate, sodium stearyl fumarate, and mixtures of magnesium stearate with sodium lauryl sulphate. Lubricants generally comprise from 0.25 weight % to 10 weight %, preferably from 0.5 weight % to 3 weight % of the tablet.

Other possible ingredients include anti-oxidants, colorants, flavoring agents, preservatives and taste-masking agents.

Tablet blends may be compressed directly or by roller to form tablets. Tablet blends or portions of blends may alternatively be wet-, dry-, or melt-granulated, melt congealed, or extruded before tabletting. The final formulation may comprise one or more layers and may be coated or uncoated; it may even be encapsulated.

The formulation of tablets is discussed in Pharmaceutical Dosage Forms: Tablets, Vol. 1, by H. Lieberman and L. Lachman (Marcel Dekker, New York, 1980).

Consumable oral films for human or veterinary use are typically pliable water-soluble or water-swellable thin film dosage forms which may be rapidly dissolving or mucoadhesive and typically comprise a film-forming polymer, a binder, a solvent, a humectant, a plasticiser, a stabiliser or emulsifier, a viscosity-modifying agent and a solvent. Some components of the formulation may perform more than one function.

Also included in the invention are multiparticulate beads comprising a composition of the invention.

Other possible ingredients include anti-oxidants, colorants, flavourings and flavour enhancers, preservatives, salivary stimulating agents, cooling agents, co-solvents (including oils), emollients, bulking agents, anti-foaming agents, surfactants and taste-masking agents.

Films in accordance with the invention are typically prepared by evaporative drying of thin aqueous films coated onto a peelable backing support or paper. This may be done in a drying oven or tunnel, typically a combined coater dryer, or by freeze-drying or vacuuming.

Solid formulations for oral administration may be formulated to be immediate and/or modified release. Modified release formulations include delayed-, sustained-, pulsed-, controlled-, targeted and programmed release.

Other suitable release technologies such as high energy dispersions and osmotic and coated particles are known.

5.2. Mucosal Administration

The compositions of the invention may also be administered to the mucosa. For example, the compositions can be administered to mucosal cells or tissue of the gastroinstinal tract, including but not limited to mucosal cells or tissues of the small intestine and/or large intestine and/or colon. Other target mucosal cells or tissues include, but are not limited to ocular, airway epithelial, lung, vaginal, and bladder cells or tissues.

Typical formulations for this purpose include liquids, gels, hydrogels, solutions, creams, foams, films, implants, sponges, fibres, powders, and microemulsions.

The compounds of the invention can be administered to the mucosa intranasally or by inhalation, typically in the form of a dry powder (either alone, as a mixture, for example, in a dry blend with lactose, or as a mixed component particle) from a dry powder inhaler or as an aerosol spray from a pressurized container, pump, spray, atomiser, or nebuliser, with or without the use of a suitable propellant.

Capsules, blisters and cartridges for use in an inhaler or insufflator may be formulated to contain a powder mix of the compound of the invention, a suitable powder base such as lactose or starch and a performance modifier such as I-leucine, mannitol, or magnesium stearate.

Formulations for inhaled/intranasal administration may be formulated to be immediate and/or modified release. Modified release formulations include delayed-, sustained-, pulsed-, controlled-, targeted and programmed release.

The compounds of the invention may be administered rectally or vaginally, for example, in the form of a suppository, pessary, or enema. Cocoa butter is a traditional suppository base, but various alternatives may be used as appropriate.

Formulations for rectal/vaginal administration may be formulated to be immediate and/or modified release. Modified release formulations include delayed-, sustained-, pulsed-, controlled-, targeted and programmed release.

The compounds of the invention may also be administered directly to the eye or ear, typically in the form of drops. Other formulations suitable for ocular and aural administration include ointments, biodegradable (e.g. absorbable gel sponges, collagen) and non-biodegradable (e.g. silicone) implants, wafers, lenses and particulate systems. Formulations may also be delivered by iontophoresis.

Formulations for ocular/aural administration may be formulated to be immediate and/or modified release. Modified release formulations include delayed-, sustained-, pulsed-, controlled-, targeted, or programmed release.

### 6. Therapeutic Applications

In one embodiment, polyplex:polymer compositions of the invention may be used for therapeutic treatment. Such compositions are sometimes referred to herein as therapeutic compositions.

Therapeutic proteins of the invention, as discussed below, are produced by polyplex:polymer compositions of the invention comprising therapeutic nucleic acids. Use of the subject proteins as described below refers to use of the subject polyplex:polymer compositions to affect such protein use.

Therapeutic proteins contemplated for use in the invention have a wide variety of activities and find use in the treatment of a wide variety of disorders. The following description of therapeutic protein activities, and indications treatable with therapeutic proteins of the invention, is exemplary and not intended to be exhaustive. The term “subject” refers to an animal, with mammals being preferred, and humans being especially preferred.

A partial list of therapeutic proteins and target diseases is shown in Table 4.

TABLE 4

| LEAD | TARGET | | THERAPEUTIC |
|---|---|---|---|
| COMPOUNDS | DISEASE | FUNCTION | EFFECT |
| Insulin | Diabetes | Insulin | Improve glucose |
| | | replacement | tolerance. |
| | | | Delay/prevent |
| | | | diabetes. |
| IL-10 + insulin | Diabetes | Immune | Delay/prevent |
| gene | | modulation and | diabetes |
| | | insulin | |
| | | replacement | |
| Glucagon | Diabetes | Reduce | Improve glucose |
| antagonists | | endogenous | tolerance |
| | | glucose | |
| | | production | |
| GLP-1 | Diabetes | Stimulate growth | Improve glucose |
| | Obesity | of ß-cells, improve | tolerance. Induce |
| | | insulin sensitivity, | weight loss |
| | | suppress appetite | |
| | Nonalcoholic | Hepatic lipid | Improve insulin |
| | Steatohepatitis | metabolism | sensitivity. |
| | (NASH) | | Reduce hepatic |
| | | | steatosis |
| Leptin | Obesity | Appetite | Induce weight |
| | Diabetes | suppression and | loss. Improve |
| | | improvement of | glucose tolerance |
| | | insulin sensitivity | |
| CCK | Obesity | Appetite | Induce weight |
| | | suppression | loss |
| Growth | GH | GH replacement | Improve growth |
| Hormone (GH) | deficiencies, | | |
| | wasting and | | |
| | anti-aging | | |
| Clotting factors | Hemophilia | Clotting factors | Improve clotting |
| | | replacement | time |
| Therapeutic | Infections | Pathogen | Prevent infections |
| antibodies and | Cancer | neutralization or | or transplant |
| antibody | | immune | rejections |
| fragments/ | | modulations | |
| portions | | | |
| Inflammation | Gastrointestinal | Immune | Prevent |
| inhibitors, e.g., | organ | modulation | inflammation in |
| IL-10, TGF-β, | inflammation; | | Gastrointestinal |
| TNFα | e.g., | | organ |
| antagonists, | inflammatory | | |
| IL-17 | bowel disease | | |
| antagonists | (IBD) | | |
| PD-L1 | Ulcerative | Immune | Prevent |
| | Colitis | modulation | inflammation in |
| | | | Gastrointestinal |
| | | | organ |
| | Graft Versus | Immune | Prevent immune- |
| | Host Disease | modulation | mediated tissue |
| | | | transplant |
| | | | rejection. |
| | Non-Muscle | Immune | Activate anti- |
| | Invasive | modulation | tumor immunity. |
| | Bladder Cancer | | |
| | (NMIBC) | | |
| IL-10 | Ulcerative | Immune | Prevent |
| | Colitis | modulation | inflammation in |
| | | | Gastrointestinal |
| | | | organ |
| IL-22 | Ulcerative | Immune | Prevent |
| | Colitis | modulation | inflammation in |
| | | | Gastrointestinal |
| | | | organ |
| NRG-4 | Ulcerative | Immune | Prevent |
| | Colitis | modulation | inflammation in |
| | | | Gastrointestinal |
| | | | organ |
| Elafin | Ulcerative | Immune | Prevent |
| | Colitis | modulation | inflammation in |
| | | | Gastrointestinal |
| | | | organ |
| IL-35 | Ulcerative | Immune | Prevent |
| | Colitis | modulation | inflammation in |
| | | | Gastrointestinal |
| | | | organ |
| GLP-2 | Short Bowel | Intestinotrophic | Improve |
| | Syndrome | growth factor | gastrointestinal |
| | | | fluid absorption. |
| PGE-2 | Graft Versus | Immune | Prevent immune- |
| | Host Disease | modulation | mediated tissue |
| | | | transplant |
| | | | rejection |
| GM-SCF | Graft Versus | Immune | Prevent immune- |
| | Host Disease | modulation | mediated tissue |
| | | | transplant |
| | | | rejection |
| | Ulcerative | Immune | Prevent |
| | Colitis | modulation | inflammation in |
| | | | Gastrointestinal |
| | | | organ |
| Anti C. difficile | C. difficile | Neutralize C. | Protection |
| toxins A/B | | difficile toxins | against |
| | | | enterotoxicity |
| IL-5 | Eosinophilic | Immune | Regulate |
| | Esophagitis | modulation | eosinophil |
| | | | trafficking to the |
| | | | esophagus |
| IL-13 | Eosinophilic | Immune | Regulate |
| | Esophagitis | modulation | eosinophil |
| | | | trafficking tot he |
| | | | esophagus |
| Eotaxin-3 | Eosinophilic | Immune | Regulate |
| | Esophagitis | modulation | eosinophil |
| | | | trafficking to the |
| | | | esophagus |
| Phenylalanine | Phenylketonuria | Enzyme | Metabolism of |
| hydroxylase | (PKU) | defficiency | phenylalanine. |
| IL-2 | Non-Muscle | Immune | Activate anti- |
| | Invasive | modulation | tumor immunity. |
| | Bladder Cancer | | |
| | (NMIBC) | | |
| Anti PD-1 | Non-Muscle | Immune | Activate anti- |
| | Invasive | modulation | tumor immunity. |
| | Bladder Cancer | | |
| | (NMIBC) | | |
| Anti CTLA-4 | Non-Muscle | Immune | Activate anti- |
| | Invasive | modulation | tumor immunity. |
| | Bladder Cancer | | |
| | (NMIBC) | | |
| IL-12 | Non-Muscle | Immune | Activate anti- |
| | Invasive | modulation | tumor immunity. |
| | Bladder Cancer | | |
| | (NMIBC) | | |
| POMC | Interstitial | Activates μ-opioid | Inhibit pain in |
| | Cystitis | receptors | inflamed tissue |
| Preproenkaphalin | Interstitial | Produce | Inhibit pain in |
| | Cystitis | endogenous | inflamed tissue |
| | | opioid peptides | |
| TRPV1 | Interstitial | Modulate pain | Inhibit pain in |
| | Cystitis | receptors. | inflamed tissue |
| FGF19 | Nonalcoholic | Activate hepatic | Regulate hepatic |
| | Steatohepatitis | FGF receptor 4/b- | lipogenesis and |
| | (NASH) | Klotho complex | improve glucose |
| | | | tolerance and |
| | | | insulin |
| | | | resistance. |
| FGF21 | Nonalcoholic | Regulate lipid | Reduce hepatic |
| | Steatohepatitis | metabolism and | steatosis |
| | (NASH) | reduces hepatic | |
| | | lipid | |
| | | accumulation. | |
| Oxyntomodulin | Nonalcoholic | Agonist of | Reduce hepatic |
| | Steatohepatitis | glucagon/GLP-l | steatosis |
| | (NASH) | receptor | |
| A1AT | Alpha-1 | Immune | Improve |
| | Antitrypsin | modulation | elasticity of lung |
| | Deficiency | | tissue and |
| | | | improve |
| | | | respiratory |
| | | | function. |
| Anti TNF-alpha | Ulcerative | Immune | Prevent |
| | Colitis | modulation | inflammation in |
| | | | Gastrointestinal |
| | | | organ |

In another embodiment, therapeutic compositions of the invention comprise therapeutic nucleic acids that do not encode therapeutic proteins, e.g., therapeutic RNAs. For example, by selecting therapeutic RNAs that target genes involved in mechanisms of disease and/or undesirable cellular or physiological conditions, the subject compositions may be used in the treatment of a wide array of diseases and conditions. The subject compositions are of such character that the therapeutic RNAs used are not limited in respect of the scope of target selection. Accordingly, the subject compositions find use in any disease or condition involving a suitable target mucosal tissue.

Specific non-limiting examples of therapeutic embodiments are described below. In some cases, the therapeutic embodiments are intended to act on non-mucosal target tissues, cells, or organs. Where the therapeutic effect is non-mucosal, it is understood that the cells or tissues contacted by the polyplex:polymer compositions described herein are mucosal and the therapeutic action is distal to the mucosal target. For example, mucosal cells can be transfected to produce and secrete a hormone or other therapeutic.

6.1. Hyperglycemia and Body Mass

Therapeutic proteins include insulin and insulin analogs. Diabetes mellitus is a debilitating metabolic disease caused by absent (type 1) or insufficient (type 2) insulin production from pancreatic β-cells (Unger, R. H. et al., Williams Textbook of Endocrinology Saunders, Philadelphia (1998)). Beta-cells are specialized endocrine cells that manufacture and store insulin for release following a meal (Rhodes, et. al. J. Cell Biol. 105:145(1987)) and insulin is a hormone that facilitates the transfer of glucose from the blood into tissues where it is needed. Patients with diabetes must frequently monitor blood glucose levels and many require multiple daily insulin injections to survive. However, such patients rarely attain ideal glucose levels by insulin injection (Turner, R. C. et al. JAMA 281:2005(1999)). Furthermore, prolonged elevation of insulin levels can result in detrimental side effects such as hypoglycemic shock and desensitization of the body's response to insulin. Consequently, diabetic patients still develop long-term complications, such as cardiovascular diseases, kidney disease, blindness, nerve damage and wound healing disorders (UK Prospective Diabetes Study (UKPDS) Group, Lancet 352, 837 (1998)).

Disorders treatable by a method of the invention include a hyperglycemic condition, such as insulin-dependent (type 1) or -independent (type 2) diabetes, as well as physiological conditions or disorders associated with or that result from the hyperglycemic condition. Thus, hyperglycemic conditions treatable by a method of the invention also include a histopathological change associated with chronic or acute hyperglycemia (e.g., diabetes). Particular examples include degeneration of pancreas (β-cell destruction), kidney tubule calcification, degeneration of liver, eye damage (diabetic retinopathy), diabetic foot, ulcerations in mucosa such as mouth and gums, excess bleeding, delayed blood coagulation or wound healing and increased risk of coronary heart disease, stroke, peripheral vascular disease, dyslipidemia, hypertension and obesity.

The subject compositions are useful for decreasing glucose, improving glucose tolerance, treating a hyperglycemic condition (e.g., diabetes) or for treating a physiological disorders associated with or resulting from a hyperglycemic condition. Such disorders include, for example, diabetic neuropathy (autonomic), nephropathy (kidney damage), skin infections and other cutaneous disorders, slow or delayed healing of injuries or wounds (e.g., that lead to diabetic carbuncles), eye damage (retinopathy, cataracts) which can lead to blindness, diabetic foot and accelerated periodontitis. Such disorders also include increased risk of developing coronary heart disease, stroke, peripheral vascular disease, dyslipidemia, hypertension and obesity.

As used herein, the term “hyperglycemic” or “hyperglycemia,” when used in reference to a condition of a subject, means a transient or chronic abnormally high level of glucose present in the blood of a subject. The condition can be caused by a delay in glucose metabolism or absorption such that the subject exhibits glucose intolerance or a state of elevated glucose not typically found in normal subjects (e.g., in glucose-intolerant subdiabetic subjects at risk of developing diabetes, or in diabetic subjects). Fasting plasma glucose (FPG) levels for normoglycemia are less than about 110 mg/dl, for impaired glucose metabolism, between about 110 and 126 mg/dl, and for diabetics greater than about 126 mg/dl.

Disorders treatable by producing a protein in a gut mucosal tissue also include obesity or an undesirable body mass. Leptin, cholecystokinin, PYY and GLP-1 decrease hunger, increase energy expenditure, induce weight loss or provide normal glucose homeostasis. Thus, in various embodiments, a method of the invention for treating obesity or an undesirable body mass, or hyperglycemia, involves the use of a therapeutic nucleic acid encoding leptin, cholecystokinin, PYY or GLP-1. In another embodiment, a therapeutic RNA targeting ghrelin is used. Ghrelin increases appetite and hunger. Thus, in various embodiments, a method of the invention for treating obesity or an undesirable body mass, or hyperglycemia, involves the use of a therapeutic RNA targeting ghrelin to decrease the expression thereof. Disorders treatable also include those typically associated with obesity, for example, abnormally elevated serum/plasma LDL, VLDL, triglycerides, cholesterol, plaque formation leading to narrowing or blockage of blood vessels, increased risk of hypertension/stroke, coronary heart disease, etc.

As used herein, the term “obese” or “obesity” refers to a subject having at least a 30% increase in body mass in comparison to an age and gender matched normal subject. “Undesirable body mass” refers to subjects having 1%-29% greater body mass than a matched normal subject as well as subjects that are normal with respect to body mass but who wish to decrease or prevent an increase in their body mass.

In one embodiment, a therapeutic protein of the invention is a glucagon antagonist. Glucagon is a peptide hormone produced by β-cells in pancreatic islets and is a major regulator of glucose metabolism (Unger R. H. & Orci L. N. Eng. J. Med. 304:1518(1981); Unger R. H. Diabetes 25:136 (1976)). As with insulin, blood glucose concentration mediates glucagon secretion. However, in contrast to insulin glucagon is secreted in response to a decrease in blood glucose. Therefore, circulating concentrations of glucagon are highest during periods of fast and lowest during a meal. Glucagon levels increase to curtail insulin from promoting glucose storage and stimulate liver to release glucose into the blood. A specific example of a glucagon antagonist is [des-His1, des-Phe6, Glu9]glucagon-NH2. In streptozotocin diabetic rats, blood glucose levels were lowered by 37% within 15 min of an intravenous bolus (0.75 μg/g body weight) of this glucagon antagonist (Van Tine B. A. et. al. Endocrinology 137:3316 (1996)). In another embodiment, the invention provides a method for treating diabetes or hyperglycemia, comprising the use of a therapeutic RNA to decrease the levels of glucagon production from the pancreas.

In another embodiment, a therapeutic protein of the invention useful for treating a hyperglycemic condition or undesirable body mass (e.g., obesity) is a glucagon-like peptide-1 (GLP-1). GLP-1 is a hormone released from L-cells in the intestine during a meal which stimulates pancreatic β-cells to increase insulin secretion. GLP-1 has additional activities that make it an attractive therapeutic agent for treating obesity and diabetes. For example, GLP-1 reduces gastric emptying, suppresses appetite, reduces glucagon concentration, increases β-cell mass, stimulates insulin biosynthesis and secretion in a glucose-dependent fashion, and likely increases tissue sensitivity to insulin (Kieffer T. J., Habener J. F. Endocrin. Rev. 20:876 (2000)). Therefore, regulated release of GLP-1 in the gut to coincide with a meal can provide therapeutic benefit for a hyperglycemic condition or an undesirable body mass. GLP-1 analogs that are resistant to dipeptidyl peptidase IV (DPP IV) provide longer duration of action and improved therapeutic value. Thus, GLP-1 analogs are preferred therapeutic polypeptides. In another embodiment, the invention provides a method for treating diabetes or hyperglycemia, comprising the use of a therapeutic RNA to decrease the levels of DPP IV.

In another embodiment, a therapeutic protein of the invention useful for treating a hyperglycemic condition is an antagonist to the hormone resistin. Resistin is an adipocyte-derived factor for which expression is elevated in diet-induced and genetic forms of obesity. Neutralization of circulating resistin improves blood glucose and insulin action in obese mice. Conversely, administration of resistin in normal mice impairs glucose tolerance and insulin action (Steppan C M et. al. Nature 409:307 (2001)). Production of a protein that antagonizes the biological effects of resistin in gut can therefore provide an effective therapy for obesity-linked insulin resistance and hyperglycemic conditions. In another embodiment, the invention provides a method for treating diabetes or hyperglycemia, comprising the use of a therapeutic RNA to decrease the levels of resistin expression in adipose tissue.

In another embodiment, a therapeutic polypeptide of the invention useful for treating a hyperglycemic condition or undesirable body mass (e.g., obesity) is leptin. Leptin, although produced primarily by fat cells, is also produced in smaller amounts in a meal-dependent fashion in the stomach. Leptin relays information about fat cell metabolism and body weight to the appetite centers in the brain where it signals reduced food intake (promotes satiety) and increases the body's energy expenditure.

In another embodiment, a therapeutic polypeptide of the invention useful for treating a hyperglycemic condition or undesirable body mass (e.g., obesity) is the C-terminal globular head domain of adipocyte complement-related protein (Acrp30). Acrp30 is a protein produced by differentiated adipocytes. Administration of a proteolytic cleavage product of Acrp30 consisting of the globular head domain to mice leads to significant weight loss (Fruebis J. et al. Proc. Natl Acad. Sci USA 98:2005 (2001)).

In another embodiment, a therapeutic polypeptide of the invention useful for treating a hyperglycemic condition or undesirable body mass (e.g., obesity) is cholecystokinin (CCK). CCK is a gastrointestinal peptide secreted from the intestine in response to particular nutrients in the gut. CCK release is proportional to the quantity of food consumed and is believed to signal the brain to terminate a meal (Schwartz M. W. et. al. Nature 404:661-71(2000)). Consequently, elevated CCK can reduce meal size and promote weight loss or weight stabilization (i.e., prevent or inhibit increases in weight gain).

Regarding PYY, see for example le Roux et al., Proc Nutr Soc. 2005 May; 64(2):213-6.

6.2. Immunological Disorders

In one embodiment, a therapeutic composition of the invention possesses immunomodulatory activity. For example, a therapeutic polypeptide of the present invention may be useful in treating deficiencies or disorders of the immune system, by activating or inhibiting the proliferation, differentiation, or mobilization (chemotaxis) of immune cells. Immune cells develop through the process of hematopoiesis, producing myeloid (platelets, red blood cells, neutrophils, and macrophages) and lymphoid (B and T lymphocytes) cells from pluripotent stem cells. The etiology of these immune deficiencies or disorders may be genetic, somatic, such as cancer or some autoimmune disorders, acquired (e.g. by chemotherapy or toxins), or infectious.

A therapeutic composition of the present invention may be useful in treating deficiencies or disorders of hematopoietic cells. For example, a therapeutic polypeptide of the present invention could be used to increase differentiation or proliferation of hematopoietic cells, including the pluripotent stem cells, in an effort to treat those disorders associated with a decrease in certain (or many) types hematopoietic cells. Examples of immunologic deficiency syndromes include, but are not limited to: blood protein disorders (e.g. agammaglobulinemia, dysgammaglobulinemia), ataxia telangiectasia, common variable immunodeficiency, DiGeorge Syndrome, HIV infection, HTLV-BLV infection, leukocyte adhesion deficiency syndrome, lymphopenia, phagocyte bactericidal dysfunction, severe combined immunodeficiency (SCIDs), Wiskott-Aldrich Disorder, anemia, thrombocytopenia, or hemoglobinuria.

A therapeutic composition of the present invention may also be useful in treating autoimmune disorders. Many autoimmune disorders result from inappropriate recognition of self as foreign material by immune cells. This inappropriate recognition results in an immune response leading to the destruction of the host tissue. Accordingly, the administration of a therapeutic composition of the present invention that inhibits an immune response, particularly the proliferation, differentiation, or chemotaxis of T-cells, may be an effective therapy in preventing autoimmune disorders.

Examples of autoimmune disorders that can be treated by the present invention include, but are not limited to: Addison's Disease, hemolytic anemia, antiphospholipid syndrome, rheumatoid arthritis, dermatitis, allergic encephalomyelitis, glomerulonephritis, Goodpasture's Syndrome, Graves' Disease, Multiple Sclerosis, Myasthenia Gravis, Neuritis, Ophthalmia, Bullous Pemphigoid, Pemphigus, Polyendocrinopathies, Purpura, Reiter's Disease, Stiff-Man Syndrome, Autoimmune Thyroiditis, Systemic Lupus Erythematosus, Autoimmune Pulmonary Inflammation, Guillain-Barre Syndrome, insulin-dependent diabetes mellitus, Crohn's disease, ulcerative colitis, and autoimmune inflammatory eye disease.

Similarly, allergic reactions and conditions, such as asthma (particularly allergic asthma) or other respiratory problems, may also be treated by a therapeutic composition of the present invention. Moreover, these molecules can be used to treat anaphylaxis, hypersensitivity to an antigenic molecule, or blood group incompatibility.

A therapeutic composition of the present invention may also be used to treat and/or prevent organ rejection or graft-versus-host disease (GVHD). Organ rejection occurs by host immune cell destruction of the transplanted tissue through an immune response. Similarly, an immune response is also involved in GVHD, but, in this case, the foreign transplanted immune cells destroy the host tissues. The administration of a therapeutic composition of the present invention that inhibits an immune response, particularly the proliferation, differentiation, or chemotaxis of T-cells, may be an effective therapy in preventing organ rejection or GVHD.

Similarly, a therapeutic composition of the present invention may also be used to modulate inflammation. For example, the therapeutic polypeptide may inhibit the proliferation and differentiation of cells involved in an inflammatory response. These molecules can be used to treat inflammatory conditions, both chronic and acute conditions, including inflammation associated with infection (e.g. septic shock, sepsis, or systemic inflammatory response syndrome (SIRS)), ischemia-reperfusion injury, endotoxin lethality, arthritis, pancreatitis, complement-mediated hyperacute rejection, nephritis, cytokine or chemokine induced lung injury, inflammatory bowel disease (IBD), Crohn's disease, or resulting from over production of cytokines (e.g. TNF or IL-1.) In one embodiment, a therapeutic RNA targeted against TNFα is used in the subject compositions to treat inflammation. In another preferred embodiment, a therapeutic RNA targeted against IL-1 is used in the subject compositions to treat inflammation. siRNA therapeutic RNAs are especially preferred. Inflammatory disorders of interest for treatment in the present invention include, but are not limited to, chronic obstructive pulmonary disorder (COPD), interstitial cystitis, and inflammatory bowel disease.

6.3. Clotting Disorders

In some embodiments, a therapeutic composition of the present invention may also be used to modulate hemostatic (the stopping of bleeding) or thrombolytic activity (clot formation). For example, by increasing hemostatic or thrombolytic activity, a therapeutic composition of the present invention could be used to treat blood coagulation disorders (e.g. afibrinogenemia, factor deficiencies), blood platelet disorders (e.g. thrombocytopenia), or wounds resulting from trauma, surgery, or other causes. Alternatively, a therapeutic composition of the present invention that can decrease hemostatic or thrombolytic activity could be used to inhibit or dissolve clotting. These therapeutic compositions could be important in the treatment of heart attacks (infarction), strokes, or scarring. In one embodiment, a therapeutic polypeptide of the invention is a clotting factor, useful for the treatment of hemophilia or other coagulation/clotting disorders (e.g., Factor VIII, IX or X)

6.4. Hyperproliferative Disorders

In one embodiment, a therapeutic composition of the invention is capable of modulating cell proliferation. Such a therapeutic polypeptide can be used to treat hyperproliferative disorders, including neoplasms.

Examples of hyperproliferative disorders that can be treated by a therapeutic composition of the present invention include, but are not limited to neoplasms located in the: abdomen, bone, breast, digestive system, liver, pancreas, peritoneum, endocrine glands (adrenal, parathyroid, pituitary, testicles, ovary, thymus, thyroid), eye, head and neck, nervous (central and peripheral), lymphatic system, pelvic, skin, soft tissue, spleen, thoracic, and urogenital.

Similarly, other hyperproliferative disorders can also be treated by a therapeutic composition of the present invention. Examples of such hyperproliferative disorders include, but are not limited to: hypergammaglobulinemia, lymphoproliferative disorders, paraproteinemias, purpura, sarcoidosis, Sezary Syndrome, Waldenstron's Macroglobulinemia, Gaucher's Disease, histiocytosis, and any other hyperproliferative disease, besides neoplasia, located in an organ system listed above.

Delivery to the circulatory system provides for access of therapeutic protein to a wide variety of tissues. Alternatively, a therapeutic composition of the present invention may stimulate the proliferation of other cells that can inhibit the hyperproliferative disorder.

For example, by increasing an immune response, particularly increasing antigenic qualities of the hyperproliferative disorder or by proliferating, differentiating, or mobilizing T-cells, hyperproliferative disorders can be treated. This immune response may be increased by either enhancing an existing immune response, or by initiating a new immune response. Alternatively, decreasing an immune response may also be a method of treating hyperproliferative disorders, such as with a chemotherapeutic agent.

6.5. Infectious Disease

In one embodiment, a therapeutic composition of the present invention can be used to treat infectious disease. For example, by increasing the immune response, particularly increasing the proliferation and differentiation of B and/or T cells, infectious diseases may be treated. The immune response may be increased by either enhancing an existing immune response, or by initiating a new immune response. Alternatively, the therapeutic composition of the present invention may also directly inhibit the infectious agent, without necessarily eliciting an immune response.

Viruses are one example of an infectious agent that can cause disease or symptoms that can be treated by a therapeutic composition of the present invention. Examples of viruses, include, but are not limited to the following DNA and RNA viral families: Arbovirus, Adenoviridae, Arenaviridae, Arterivirus, Birnaviridae, Bunyaviridae, Caliciviridae, Circoviridae, Coronaviridae, Flaviviridae, Hepadnaviridae (Hepatitis), Herpesviridae (such as, Cytomegalovirus, Herpes Simplex, Herpes Zoster), Mononegavirus (e.g. Paramyxoviridae, Morbillivirus, Rhabdoviridae), Orthomyxoviridae (e.g. Influenza), Papovaviridae, Parvoviridae, Picornaviridae, Poxviridae (such as Smallpox or Vaccinia), Reoviridae (e.g. Rotavirus), Retroviridae (HTLV-I, HTLV-II, Lentivirus), and Togaviridae (e.g. Rubivirus). Viruses falling within these families can cause a variety of diseases or symptoms, including, but not limited to: arthritis, bronchiolitis, encephalitis, eye infections (e.g. conjunctivitis, keratitis), chronic fatigue syndrome, hepatitis (A, B, C, E, Chronic Active, Delta), meningitis, opportunistic infections (e.g. AIDS), pneumonia, Burkitt's Lymphoma, chickenpox, hemorrhagic fever, Measles, Mumps, Parainfluenza, Rabies, the common cold, Polio, leukemia, Rubella, sexually transmitted diseases, skin diseases (e.g. Kaposi's, warts), and viremia. A therapeutic composition of the present invention can be used to treat any of these symptoms or diseases.

Similarly, bacterial or fungal agents that can cause disease or symptoms and that can be treated by a therapeutic composition of the present invention include, but are not limited to, the following Gram-Negative and Gram-positive bacterial families and fungi: Actinomycetales (e.g. Corynebacterium, Mycobacterium, Norcardia), Aspergillosis, Bacillaceae (e.g. Anthrax, Clostridium), Bacteroidaceae, Blastomycosis, Bordetella, Borrelia, Brucellosis, Candidiasis, Campylobacter, Coccidioidomycosis, Cryptococcosis, Dermatocycoses, Enterobacteriaceae (Klebsiella, Salmonella, Serratia, Yersinia), Erysipelothrix, Helicobacter, Legionellosis, Leptospirosis, Listeria, Mycoplasmatales, Neisseriaceae (e.g. Acinetobacter, Gonorrhea, Menigococcal), Pasteurellacea Infections (e.g. Actinobacillus, Heamophilus, Pasteurella), Pseudomonas, Rickettsiaceae, Chlamydiaceae, Syphilis, and Staphylococcal. These bacterial or fungal families can cause the following diseases or symptoms, including, but not limited to: bacteremia, endocarditis, eye infections (conjunctivitis, tuberculosis, uveitis), gingivitis, opportunistic infections (e.g. AIDS related infections), paronychia, prosthesis-related infections, Reiter's Disease, respiratory tract infections, such as Whooping Cough or Empyema, sepsis, Lyme Disease, Cat-Scratch Disease, Dysentery, Paratyphoid Fever, food poisoning, Typhoid, pneumonia, Gonorrhea, meningitis, Chlamydia, Syphilis, Diphtheria, Leprosy, Paratuberculosis, Tuberculosis, Lupus, Botulism, gangrene, tetanus, impetigo, Rheumatic Fever, Scarlet Fever, sexually transmitted diseases, skin diseases (e.g. cellulitis, dermatocycoses), toxemia, urinary tract infections, wound infections. A therapeutic composition of the present invention can be used to treat any of these symptoms or diseases.

Moreover, parasitic agents causing disease or symptoms that can be treated by a therapeutic composition of the present invention include, but are not limited to, the following families: Amebiasis, Babesiosis, Coccidiosis, Cryptosporidiosis, Dientamoebiasis, Dourine, Ectoparasitic, Giardiasis, Helminthiasis, Leishmaniasis, Theileriasis, Toxoplasmosis, Trypanosomiasis, and Trichomonas. These parasites can cause a variety of diseases or symptoms, including, but not limited to: Scabies, Trombiculiasis, eye infections, intestinal disease (e.g. dysentery, giardiasis), liver disease, lung disease, opportunistic infections (e.g. AIDS related), Malaria, pregnancy complications, and toxoplasmosis. A therapeutic composition of the present invention can be used to treat any of these symptoms or diseases.

6.6. Regeneration

A therapeutic composition of the present invention can be used to differentiate, proliferate, and attract cells, fostering the regeneration of mucosal tissues or tissues adjacent to the target mucosal cells or tissues. (See, Science 276:59-87 (1997).) The regeneration of tissues could be used to repair, replace, or protect tissue damaged by congenital defects, trauma (wounds, burns, incisions, or ulcers), age, disease (e.g. osteoporosis, osteoarthritis, periodontal disease, liver failure), surgery, including cosmetic plastic surgery, fibrosis, reperfusion injury, or systemic cytokine damage.

Therapeutic compositions of the invention may promote the regeneration of a variety of tissues, including but not limited to organs (e.g. pancreas, liver, intestine, kidney, skin, endothelium), muscle (smooth, skeletal or cardiac), vascular (including vascular endothelium), nervous, hematopoietic, and skeletal (bone, cartilage, tendon, and ligament) tissue. Preferably, regeneration incurs a small amount of scarring, or occurs without scarring. Regeneration also may include angiogenesis.

Moreover, a therapeutic composition of the present invention may increase regeneration of tissues difficult to heal. For example, increased tendon/ligament regeneration would quicken recovery time after damage. A therapeutic composition of the present invention could also be used prophylactically in an effort to avoid damage. Specific diseases that could be treated include tendinitis, carpal tunnel syndrome, and other tendon or ligament defects. A further example of tissue regeneration of non-healing wounds includes pressure ulcers, ulcers associated with vascular insufficiency, surgical, and traumatic wounds.

Similarly, nerve and brain tissue could also be regenerated by using a therapeutic composition of the present invention to proliferate and differentiate nerve cells. Diseases that could be treated using this method include central and peripheral nervous system diseases, neuropathies, or mechanical and traumatic disorders (e.g. spinal cord disorders, head trauma, cerebrovascular disease, and stoke). Specifically, diseases associated with peripheral nerve injuries, peripheral neuropathy (e.g. resulting from chemotherapy or other medical therapies), localized neuropathies, and central nervous system diseases (e.g. Alzheimer's disease, Parkinson's disease, Huntington's disease, amyotrophic lateral sclerosis, and Shy-Drager syndrome), could all be treated using therapeutic compositions of the present invention. With respect to CNS disorders, numerous means are known in the art for facilitating therapeutic access to brain tissue, including methods for disrupting the blood brain barrier, and methods of coupling therapeutic agents to moieties that provide for transport into the CNS. In one embodiment, a therapeutic nucleic acid is engineered so as to encode a fusion protein, which fusion protein comprises a transport moiety and a therapeutic protein.

6.7. Chemotaxis

In one embodiment, a therapeutic composition of the invention can modulate chemotaxis. For example, in one embodiment, a therapeutic polypeptide of the present invention possesses a chemotaxis activity. A chemotaxic molecule attracts or mobilizes cells (e.g. monocytes, fibroblasts, neutrophils, T-cells, mast cells, eosinophils, epithelial and/or endothelial cells) to a particular site in the body, such as inflammation, infection, or site of hyperproliferation. The mobilized cells can then fight off and/or heal the particular trauma or abnormality.

For example, a therapeutic polypeptide of the present invention may increase chemotaxic activity of particular cells. These chemotaxic molecules can then be used to treat inflammation, infection, hyperproliferative disorders, or any immune system disorder by increasing the number of cells targeted to a particular location in the body. For example, chemotaxic molecules can be used to treat wounds and other trauma to tissues by attracting immune cells to the injured location. Chemotaxic molecules of the present invention can also attract fibroblasts, which can be used to treat wounds.

It is also contemplated that a therapeutic composition of the present invention may inhibit chemotaxic activity. These therapeutic compositions could also be used to treat disorders. Thus, a therapeutic composition of the present invention could be used as an inhibitor of chemotaxis.

Especially preferred for use are protherapeutic proteins that are activated in the vicinity of target tissues.

Additional therapeutic polypeptides contemplated for use include, but are not limited to, growth factors (e.g., growth hormone, insulin-like growth factor-1, platelet-derived growth factor, epidermal growth factor, acidic and basic fibroblast growth factors, transforming growth factor-β, etc.), to treat growth disorders or wasting syndromes; and antibodies (e.g., human or humanized), to provide passive immunization or protection of a subject against foreign antigens or pathogens (e.g., H. Pylori), or to provide treatment of cancer, arthritis or cardiovascular disease; cytokines, interferons (e.g., interferon (IFN), IFN-α2b and 2a, IFN-α N1, IFN-β1b, IFN-gamma), interleukins (e.g., IL-1 to IL-10), tumor necrosis factor (TNF-α TNF-β), chemokines, granulocyte macrophage colony stimulating factor (GM-CSF), polypeptide hormones, antimicrobial polypeptides (e.g., antibacterial, antifungal, antiviral, and/or antiparasitic polypeptides), enzymes (e.g., adenosine deaminase), gonadotrophins, chemotactins, lipid-binding proteins, filgastim (Neupogen), hemoglobin, erythropoietin, insulinotropin, imiglucerase, sarbramostim, tissue plasminogen activator (tPA), urokinase, streptokinase, phenylalanine ammonia lyase, brain-derived neurotrophic factor (BDNF), nerve growth factor (NGF), thrombopoietin (TPO), superoxide dismutase (SOD), adenosine deamidase, catalase, calcitonin, endothelin, L-asparaginase pepsin, uricase, trypsin, chymotrypsin, elastase, carboxypeptidase, lactase, sucrase, intrinsic factor, parathyroid hormone (PTH)-like hormone, soluble CD4, and antibodies and/or antigen-binding fragments (e.g, FAbs) thereof (e.g., orthoclone OKT-e (anti-CD3), GPIIb/IIa monoclonal antibody). Additionally contemplated are therapeutic RNAs targeting nucleic acids encoding such factors.

6.8. Vaccine

In one embodiment, the invention provides methods for vaccinating a patient. The methods comprise administering a composition of the invention capable of producing the desired epitope. In a preferred embodiment, the composition comprises a therapeutic nucleic acid construct capable of expressing a protein comprising the epitope.

6.9. Cosmetic Applications

In one embodiment, the invention provides DD-chitosan nucleic acid polyplexes for cosmetic use. The subject cosmetics comprise DD-chitosan nucleic acid polyplexes in a formulation suitable for cosmetic use.

### EXAMPLES

### Example 1

### 1. General Materials

1.1 Plasmid DNA vectors

| Plasmid DNA name | Comments |
|---|---|
| pVax-opt-hIL10 | pVax backbone with human interleukin-10 gene |
| | coupled to CMV promoter |
| pVax-PD-L1-Fc | pVax backbone with PD-L1 + Fc gene coupled to |
| | CMV promoter |
| gWiz-GFP | gWIZ backbone with GFP gene |
| pVax2 | pVax backbone control |

1.2 Reagents

| Material | Cat. No. |
|---|---|
| PEG-polyglutamic acid (PEG-PGA), | mPEG1K-b-PLE10 |
| mPEG_{1K}-b-PLE₁₀, average MW 2500 | |
| PEG-polyglutamic acid (PEG-PGA) | mPEG5K-b-PLE10 |
| mPEG_{5K}-b-PLE₁₀, average MW 6500 | |
| PEG-polyglutamic acid (PEG-PGA) | mPEG5K-b-PLE50 |
| mPEG_{5K}-b-PLE₅₀, average MW 13000 | |
| PEG-hyaluronic acid (PEG-HA), | HA-201 |
| HA MW 10k & PEG MW 2k | |
| PEG-hyaluronic acid (PEG-HA), | HA-202 |
| HA MW 50k & PEG MW 2k | |
| PEG-polyaspartic acid (PEG-PAA) | mPEG5K-b-PLD10 |
| mPEG_{5K}-b-PLD₁₀, average MW 6400 | |
| Sodium chloride | BP-358-212 |
| MilliQ water (Type 1 water) | NA |
| DLS disposable cuvettes | 759075D |
| Zeta potential cuvettes | DTS1070 |
| Trehalose | BP2687 |
| Trehalose | T-104-4 |
| UltraPure Agarose | 16500-100 |
| TBE Buffer | 15581-028 |
| 10,000X SYBRSafe DNA Stain | S33102 |
| 6X Loading Buffer | n/a |
| Supercoil DNA Ladder | N0472S |
| Poly-(α,β)-DL-aspartic acid sodium | P3418-1G |
| salt (PAA) | |
| Tris buffer | N/A |
| 10X CutSmart | B7204S |
| NotI-HF | R3189L |
| Quant-it-Picogreen dsDNA reagent | P7589 |
| 1X TBE | P7589 |
| Nuclease Free water | AM9932 |
| 1.0M NaOH | RI745016 |
| DMEM | 11995-065 |
| FaSSIF V1 | FFF01 |
| FaSSIF V2 | V2FAS01 |
| Polystyrene beads amine modified | L9904-1ML |
| (100 nm, orange) | |
| Polystyrene beads amine modified | F8764 |
| (200 nm, green) | |
| Polystyrene beads, amine modified | L0780 |
| (50 nm, blue) | |
| PEG-NHS, 5 kD | PGl-SC-5k |
| EDC-HCl | 4031236 |
| Hydrochloric acid | SA48-500 |

1.3 Consumables

| Material | Cat. No. |
|---|---|
| Pipette tips P1250 | 10017-216 |
| Pipette tips P300 | M-0300-9FC |
| 96-well plates, Black, Flat bottom, untreated | 3915 |
| Syringe filter, 32 mm 0.2 μm | REF 4652 |
| Syringe filter, 13 mm 0.2 μm | REF 4602 |
| Syringe | REF305180 |
| Blunt needle | REF302830 |
| Centrifuge tubes | 4488 |
| 0.2 μm PES Bottle-top Filter | 431097 |
| Pt-cured Si tubing, size L/S 14 (BioPharm) | 96420-14 |
| Pt-cured Si tubing size L/S 16 | 96410-16 |
| PharmaPure tubing size L/S 16 | 06435-16 |
| Peroxide-cured Si tubing, size L/S 14 | 96400-14 |
| Micro centrifuge tubes | MCT-150-C |
| Amicon ultra 0.5 mL | UFC503024 |
| Scinitllation vial-7 mL with cap | 03-337-26 |

1.4 Equipment

| Description | Manufacturer |
|---|---|
| DLS-Zeta sizer | Malvern |
| Water Bath, IsoTemp 210 | Fisher Scientific |
| Pipette P1000 | Eppendorf |
| Pipette P200 | Eppendorf |
| Balance, AG104, 101 mg | Mettler |
| Balance. W3200 | Accuris Instruments |
| Vortex, Genie 2 | Fisher |
| Sonicator | Branson |
| Stirrer/hot plate, model 6795-420D | Corning |
| Centrifuge, model 5417C | Eppendorf |
| Mini Centrifuge, my SPIN | Fisher Scientific |
| Gel Box Station, Midi Plus 15 | VWR |
| Gel Electrophoresis Power Source, EC 200 XL | Thermo Scientific |
| Gel Imaging Station, GelDoc System UV | BioRad |
| Transilluminator + Quantity One quantitation | |
| software | |
| Top Load Balance, W3200-3200 | Accuris Instruments |
| Spectrophotometer, SpectraMax M2 | Molecular Devices |
| NanoDrop One Spectrometer | Thermo Scientific |
| Syringe Pump, NE-1000 | New Era Pump Systems |
| L/S Digi-Staltic Pump System, Controller | Cole Parmer |
| L/S Digi-Staltic Pump System, Pump | Cole Parmer |
| L/S/ Pumphead | Cole Parmer |
| pH Meter, Orion Star A211 | Thermo Scientific |

### 2. General Procedures

2.1 Preparation of Dually Derivatized Chitosan

Chitosan was dually derivatized (DD-chitosan, DD-X) with arginine and gluconic acid according to U.S. Pat. No. 9,623,112 B2.

2.2 Preparation of Dually Derivatized Chitosan and DNA Polyplexes

DD-chitosan was polyplexed with a plasmid DNA vector according to U.S. Pat. No. 9,623,112B2 and U.S. Pat. No. 8,722,646B2 at various amine-to-phosphate (N:P) molar ratios, as required. Additional excipient such as sucrose, trehalose or mannitol were included as required. Various plasmid DNA vectors were tested as indicated herein.

2.3 Preparation of PEG-Polyglutamic Acid (PEG-PGA) Solution

Generally, PEG-PGA was dissolved in water or excipient solution as required at a concentration of up to 40 mg/mL. The resulting PEG-PGA solution was diluted to required molar concentration of anionic species (A, i.e. glutamic acid) necessary, in order to attain the desired final ratio of amine-to-phosphate-to-anion molar ratio (N:P:A) for subsequent formulations.

2.4 Preparation of PEG-Hyaluronic Acid (PEG-HA) Solution

Generally, PEG-HA was dispensed in water at a concentration of 40 to 100 mg/mL, and sonicated for 10 minutes. The resulting PEG-HA solution was diluted to required concentration of anionic species (A, i.e. hyaluronic acid) in 10% trehalose necessary in order to attain the desired final ratio of amine-to-phosphate-to-anion molar ratio (N:P:A) for subsequent formulations, and such that final concentration of trehalose was 5%.

2.5 Preparation of PEG-Polyaspartic Acid (PEG-PAA) Solution

Generally, PEG-PAA was dispensed in 5% trehalose at a concentration of 40 to 100 mg/mL, and sonicated for 10 to 15 minutes. If required, the resulting PEG-PAA solution was diluted to required concentration of anionic species (A, i.e. aspartic acid) in 5% trehalose necessary in order to attain the desired final ratio of amine-to-phosphate-to-anion molar ratio (N:P:A) for subsequent formulations, and such that final concentration of trehalose was 5%.

2.6 Preparation of Trehalose Solution

Generally, trehalose was dissolved in water at a concentration of up to 0.2 g/mL as required. The resulting trehalose filtered using a 0.2 μm filter. If necessary, the trehalose solution was diluted to required concentration necessary for subsequent formulations.

2.7 Preparation of PAA Solution in 50 mM Tris, pH 8

Generally, PAA was dissolved in 50 mM Tris pH 8 at a concentration of 100 mg/mL or 20 mg/mL as required. If necessary, the PAA solution was diluted in 50 mM Tris pH 8 to required concentration necessary for subsequent need.

2.8 Preparation of 20 mM NaCl

Dissolve 46.75 mg of NaCl in 40 mL of Water. Filter the solution through a 0.2 um filter

2.9 Preparation of Simulated Intestinal Fluids (SIF)

FaSSIF V1 solution was prepared by dissolving FaSSIF V1 powder in water at a concentration of 2.24 mg/mL. pH was verified to be approximately 6.6. FaSSIF V2 solution was prepared by dissolving FaSSIF V2 powder in water at a concentration of 1.79 mg/mL. pH was verified to be approximately 6.6

2.10 PEG-Polyanion (PEG-PA)

PEG-PA is a general term for PEG conjugated to a polyanion such as PEG-polyglutamic acid, PEG-Hyaluronic acid, or PEG-polyaspartic acid.

2.11 Preparation of PEGylated Polyplexes by Dripping

PEGylated polyplexes are prepared by dripping equal volume of polyplex solution (0.1 mL) into diluted PEG-PA solution (0.1 mL).

2.12 Preparation of PEGylated Polyplexes by In-Line Mixing

PEGylated polyplexes are prepared by mixing equal volume of diluted PEG-PA solution with polyplex solution using an in-line mixing apparatus such as described in U.S. Pat. No. 9,623,112B2 and U.S. Pat. No. 8,722,646B2.

2.13 Stability of PEGylated Polyplexes in 150 Mmol PBS

Mix 100 uL of PEGylated Polyplexes in 300 uL of 150 Mmol PBS. Measure particle size diameter at 0 and 2 h of mixing. Compare with non-PEGylated polyplex controls.

2.14 Percent supercoiled DNA

Prior to percent supercoil DNA measurement, total DNA must be released from the polyplex or PEGylated polyplex by subjecting it to excess PAA. Samples aliquots of 1 μL (target of 0.1 mg/mL DNA) were combined with 10 μL of PAA (100 mg/mL in 50 mM Tris), mixed, and incubated at 37° C. for 30 min. Following release, DNA is subjected to agarose gel electrophoresis.

2.15 Total DNA in Formulation by Picogreen

Prior to DNA measurement using the PicoGreen assay, total DNA must be released from the polyplex or PEGylated polyplex using excess PAA. Sample aliquots of 10 μL (target of 2 ug/mL DNA) were combined with 10 μL of PAA (20 mg/mL in 50 mM Tris), mixed, and incubated at 37° C. for 30 min. Following release, DNA is subjected to digestion using a suitable restriction enzyme (RE) to linearize the supercoiled DNA plasmids. For picogreen assay, a 10 uL aliquot of the RE digested sample is mixed with 190 uL of picogreen working solution (Qubit ds DNA buffer:Qubit ds DNA Picogreen reagent 199:1), incubated for 2 minutes, and measured for fluorescence (Excitation: 485, Emission cutoff: 515 nm, Emission: 535 nm). The results are quantified against a reference DNA standard curve.

2.16 Free DNA

For verification of DNA capture into the polyplex, samples aliquots of 10 μL (target of 1000 ng DNA) were combined with 2 μL of 6× loading buffer and subjected to gel electrophoresis.

2.17 Gel Electrophoresis

Sample lanes were loaded with prepared samples as described. Standard lanes were loaded with Supercoiled DNA ladder. Reference lanes were loaded with 2 μL, of reference DNA (200 ng of DNA)+8 μL water+2 μL 6× loading buffer. The samples were resolved on a 0.8% agarose gel containing 1×SYBRSafe DNA Stain at 100 V for 75 minutes. The gel was imaged with the GelDoc Imaging System.

2.18 Nanoparticle Sizing of Polyplexes and PEGylated Polyplexes

Particle size measurements were made using a Zetasizer Nano light scattering instrument. In general, samples were either undiluted or diluted up to 20-fold in 10 mM NaCl and loaded into a disposable cuvette or a Zetasizer folded capillary cell (0.8 mL minimum). The Zetasizer was programmed to incubate the sample for up to 3 minutes at 25° C. prior to triplicate 3-minute measurements. Z-average diameter and polydispersity (PDI) were reported with standard deviation (n=3). The Zetasizer was also programmed to account for the composition of the samples with regards to viscosity and refractive index.

2.19 Zeta Potential of Polyplexes and PEGylated Polyplexes

Zeta potential measurements were made using a Zetasizer Nano light scattering instrument. In general, undiluted samples were loaded into a Zetasizer folded capillary cell (0.8 mL minimum), except PEGylated polyplexes which were diluted in 10 mM NaCl. The Zetasizer was programmed to incubate the sample for up to 3 minutes at 25° C. prior to replicate measurements (number of replicates were automatically determined by Zetasizer software). Zeta potential values were reported with standard deviation (n=3). The Zetasizer was also programmed to account for the final composition of the samples with regards to viscosity and dielectric constant.

2.20 Short-Term Stability

For short-term stability studies, formulations were freeze-dried as described and stored at the appropriate temperature (−20° C., 4° C. or room temperature). At the appropriate times, samples were rehydrated and analyzed as described.

3. PEGylation of Polyplexes Using PEG-PGA (mPEG1K-b-PLE10)

3.1 Procedure

DD-X—DNA (pVax) polyplexes were produced at N:P ratio as indicated below and DNA concentration of 0.1 mg/mL DNA with 5% trehalose as previously described.

| Sample ID | N:P ratio | Comment |
|---|---|---|
| CMC-INT05-003 NP3.3 | 3:1 | DD-X—DNApolyplex, |
| CMC-INT05-003 NP5 | 5:1 | 5% trehalose, 0.1 mg/mL |
| CMC-INT05-003 NP7 | 7:1 | |

PEG-PGA solution was then mixed with DD-X-DNA polyplexes at a 1:1 volume ratio by drip mixing to yield PEGylated polyplex at 0.05 mg/mL DNA as indicated below. Samples were tested for particle size, PDI and zeta potential (tested formulations were not frozen).

| Source Polyplex | PEG-PGA added | N:P:A Ratio |
|---|---|---|
| CMC-INT05-003 | mPEG1K-b-PLE10 | 3:1:1.1 |
| NP3.3 | | 3:1:5.5 |
| | | 3:1:11 |
| | | 3:1:21 |
| | | 3:1:53 |
| CMC-INT05-003 | mPEG1K-b-PLE10 | 5:1:1.1 |
| NP5 | | 5:1:5.5 |
| | | 5:1:11 |
| | | 5:1:21 |
| | | 5:1:53 |
| CMC-INT05-003 | mPEG1K-b-PLE10 | 7:1:1.1 |
| NP7 | | 7:1:5.5 |
| | | 7:1:11 |
| | | 7:1:21 |
| | | 7:1:53 |

3.2 Results

Non-freeze-thawed PEGylated polyplexes made with mPEG1K-b-PLE10 yielded stable formulations (except as indicated) at certain N:P:A ratios as shown below.

| Sample | PEG-PGA | N:P:A | Particle Size | | Zeta Potential |
|---|---|---|---|---|---|
| ID | added | Ratio | (nm) | PDI | (mV) |
| CMC- | mPEG1K-b- | 3:1:0 | 193.83 | 0.14 | 30 |
| INT05- | PLE10 | 3:1:1.1 | * | * | * |
| 003 | | 3:1:5.5 | 8761.00 * | 0.35 | 4 |
| NP3.3 | | 3:1:11 | 200.30 | 0.10 | −12 |
| | | 3:1:21 | 199.20 | 0.15 | −28 |
| | | 3:1:53 | 200.73 | 0.16 | −31 |
| CMC- | mPEG1K-b- | 5:1:0 | 138.83 | 0.20 | 36 |
| INT05- | PLE10 | 5:1:1.1 | * | * | * |
| 003 | | 5:1:5.5 | 222.53 | 0.07 | 17 |
| NP5 | | 5:1:11 | 10946.33 * | 0.82 | 6 |
| | | 5:1:21 | 164.60 | 0.11 | −17 |
| | | 5:1:53 | 135.77 | 0.14 | −27 |
| CMC- | mPEG1K-b- | 7:1:0 | 137.80 | 0.18 | 37 |
| INT05- | PLE10 | 7:1:1.1 | ND | ND | ND |
| 003 | | 7:1:5.5 | 136.87 | 0.15 | 20 |
| NP7 | | 7:1:11 | 1246.67 * | 0.39 | 17 |
| | | 7:1:21 | 2582.00 * | 1.00 | −3 |
| | | 7:1:53 | 173.00 | 0.09 | −25 |

* Aggregated samples

4. PEGylation of Polyplexes Using PEG-PGA (mPEG1K-b-PLE10 and mPEG5K-b-PLE10) and PEG-HA (HA-202)

4.1 Procedure

DD-X—DNA (pVax-opt-hIL10) polyplexes were produced at N:P ratio as indicated below and DNA concentration of 0.25 mg/mL DNA with 5% trehalose as previously described.

| Sample ID | N:P ratio | Comment |
|---|---|---|
| CMC-INT06-024 NP3 | 3:1 | DD-X—DNA polyplex, 5% trehalose, |
| CMC-INT06-024 NP5 | 5:1 | 0.25 mg/mL |
| CMC-INT06-024 NP7 | 7:1 | |

PEG-PGA solution or PEG-HA solution was then mixed with DD-X-DNA polyplexes at a 1:1 volume ratio by drip mixing to yield PEGylated polyplex at 0.125 mg/mL DNA as indicated below. Samples were frozen and thawed, then tested for particle size, PDI and zeta potential.

| Sample ID | Source Polyplex | PEG-PGA added | N:P:A Ratio * |
|---|---|---|---|
| CMC-INT06- | CMC-INT06- | mPEG1K-b-PLE10 | 3:1:276 |
| EXP-034-A | 024 NP3 | | |
| CMC-INT06- | CMC-INT06- | | 5:1:460 |
| EXP-034-B | 024 NP5 | | |
| CMC-INT06- | CMC-INT06- | | 7:1:644 |
| EXP-034-C | 024 NP7 | | |
| CMC-INT06- | CMC-INT06- | mPEG5K-b-PLE10 | 3:1:25.5 |
| EXP-034-D | 024 NP3 | | |
| CMC-INT06- | CMC-INT06- | | 5:1:42.5 |
| EXP-034-E | 024 NP5 | | |
| CMC-INT06- | CMC-INT06- | | 7:1:59.5 |
| EXP-034-F | 024 NP7 | | |
| CMC-INT06- | CMC-INT06- | HA-202 | 3:1:45 |
| EXP-034-G | 024 NP3 | | |
| CMC-INT06- | CMC-INT06- | | 5:1:75 |
| EXP-034-H | 024 NP5 | | |
| CMC-INT06- | CMC-INT06- | | 7:1:105 |
| EXP-034-I | 024 NP7 | | |

* N:A ratio set to: 1:92 for mPEG1K-b-PLE10, 1:8.5 for mPEG5K-b-PLE10, and 1:15 for HA-202.

4.2 Results

Physicochemical results of freeze-thawed samples are provided in table below. PEGylated polyplexes made with mPEG1K-b-PLE10 or HA-202 did not yield formulations stable to freeze-thaw. PEGylated polyplexes made with mPEG5K-b-PLE10 were stable to freeze-thaw.

| | PEG- | | Particle | | Zeta |
|---|---|---|---|---|---|
| | PGA | N:P:A | Size | | Potential |
| Sample ID | added | Ratio | (nm) | PDI | (mV) ** |
| CMC-INT06-024 NP3 | None | 3:1:0 | 194 | 0.14 | 30 |
| CMC-INT06-024 NP5 | | 5:1:0 | 139 | 0.20 | 36 |
| CMC-INT06-024 NP7 | | 7:1:0 | 138 | 0.18 | 37 |
| CMC-INT06-EXP-034-A | mPEG1K- | 3:1:276 | 1852 * | 0.69 | −30 |
| CMC-INT06-EXP-034-B | b-PLE10 | 5:1:460 | 1163 * | 0.53 | −30 |
| CMC-INT06-EXP-034-C | | 7:1:644 | 1712 * | 0.64 | −29 |
| CMC-INT06-EXP-034-D | mPEG5K- | 3:1:25.5 | 214 | 0.13 | −4 |
| CMC-INT06-EXP-034-E | b-PLE10 | 5:1:42.5 | 158 | 0.18 | −3 |
| CMC-INT06-EXP-034-F | | 7:1:59.5 | 155 | 0.17 | −3 |
| CMC-INT06-EXP-034-G | HA-202 | 3:1:45 | 665 * | 0.46 | −19 |
| CMC-INT06-EXP-034-H | | 5:1:75 | 485 * | 0.41 | −17 |
| CMC-INT06-EXP-034-I | | 7:1:105 | 497 * | 0.37 | −16 |

* Aggregated samples
** Zeta potential for PEGylated samples was determined on fresh samples (not freeze-thawed).

5. PEGylation of Polyplexes with PEG-HA

5.1 Procedure

DD-X—DNA (pVax-opt-hIL10) polyplexes were produced at N:P ratio as indicated below and DNA concentration of 0.25 mg/mL DNA with 5% trehalose as previously described.

| Sample ID | N:P ratio | Comment |
|---|---|---|
| CMC-INT06-024 NP3 | 3:1 | DD-X-DNA polyplex, 5% |
| CMC-INT06-024 NP5 | 5:1 | trehalose, 0.25 mg/mL |
| CMC-INT06-024 NP7 | 7:1 | |

PEG-HA solution in 5% trehalose was then mixed with DD-X-DNA polyplexes at a 1:1 volume ratio by drip mixing to yield PEGylated polyplex at 0.125 mg/mL DNA as indicated below. Samples were tested for particle size, PDI and zeta potential.

| Source Polyplex | PEG-HA added | N:P:A Ratio |
|---|---|---|
| CMC-INT06-024 NP3 | HA-201 | Not applicable. |
| | (HA 10k-PEG 2k) | See results for |
| CMC-INT06-024 NP5 | HA-201 | HA-201. |
| | (HA 10k-PEG 2k) | |
| CMC-INT06-024 NP7 | HA-201 | |
| | (HA 10k-PEG 2k) | |

| Source Polyplex | PEG-HA added | N:P:A Ratio |
|---|---|---|
| CMC-INT06- | HA-202 | 5:1:0.4 |
| 024 NP5 | (HA 50k-PEG 2k) | 5:1:1 |
| | | 5:1:2 |
| CMC-INT06- | HA-202 | 7:1:0.4 |
| 024 NP7 | (HA 50k-PEG 2k) | 7:1:1 |
| | | 7:1:2 |

5.2 Results

HA-201 (HA 10 k-PEG 2 k) was not soluble in water. It formed hydrogel and was not used further. HA-202 (HA 50 k-PEG 2 k) formed a viscous solution in water. Polyplexes showed visible aggregation after PEGylation, and no further testing was performed.

### 6. Preparation of PEG-PGA Polyplexes

6.1 Procedure

### Preparation of PEGylated Polyplexes

N:P 7 polyplex (CMC-INT06-098A) aliquots were PEGylated using PEG-PGA as described herein. The resulting PEGylated polyplexes were tested for physicochemical properties and DNA capture. The resulting formulations in addition to the N:P 7 polyplex, were generated.

| | Nominal | N:P:A | |
|---|---|---|---|
| Sample Lot. | [DNA] | Ratio | Additional description |
| CMC-INT06-098 A | 0.25 mg/mL | 7:1:0 | Control, DD-X-DNA |
| | | | (pVax-PD-L1-Fc) |
| | | | polyplex, 5% trehalose |
| CMC-INT06-098 B | 0.125 mg/mL | 7:1:(0.88) | PEGylated with PEG- |
| CMC-INT06-098 C | 0.125 mg/mL | 7:1:(1.23) | PGA (mPEG5K-b- |
| CMC-INT06-098 D | 0.125 mg/mL | 7:1:(1.75) | PLE10), 5% trehalose |
| CMC-INT06-098 E | 0.125 mg/mL | 7:1:(3.5) | |
| CMC-INT06-098 F | 0.125 mg/mL | 7:1:(3.5) | PEGylated with PEG- |
| | | | PGA (mPEG5K-b- |
| | | | PLE10), 2.5% |
| | | | Trehalose |

6.2 Results

The appearance, pH, particle size, PDI, zeta potential, and conductivity of the test samples were:

| | | Particle | | Zeta | |
|---|---|---|---|---|---|
| | | Size | | Potential | |
| Entity ID | Appearance | (nm) | PDI | (mV) | pH |
| CMC-INT06-098 A | Clear/ | 132 | 0.192 | 31.4 | 6.09 |
| | translucent | | | | |
| CMC-INT06-098 B | Clear/ | 136 | 0.196 | 16.2 | 5.96 |
| | translucent | | | | |
| CMC-INT06-098 C | Clear/ | 136 | 0.173 | Not | 6.14 |
| | translucent | | | determined | |
| CMC-INT06-098 D | Clear/ | 137 | 0.172 | Not | 6.03 |
| | translucent | | | determined | |
| CMC-INT06-098 E | Clear/ | 139 | 0.150 | 4.5 | 6.2 |
| | translucent | | | | |
| CMC-INT06-098 F | Clear/ | 139 | 0.157 | Not | 6.25 |
| | translucent | | | determined | |

The Free DNA and % Supercoil were:

| | | Supercoil Content |
|---|---|---|
| Entity ID | Free DNA | (%) |
| CMC-INT06-098 A | Non-visible | 94 |
| CMC-INT06-098 B | Non-visible | 93 |
| CMC-INT06-098 C | Non-visible | 91 |
| CMC-INT06-098 D | Non-visible | 92 |
| CMC-INT06-098 E | Non-visible | 93 |
| CMC-INT06-098 F | Non-visible | 93 |

7. Concentrated PEGylated Polyplex by In-Line Mixing with PEG-PGA Followed by TFF Concentration

7.1 Procedure

Concentrated PEGylated Polyplex by In-Line Mixing with PEG-PGA Followed by TFF Concentration

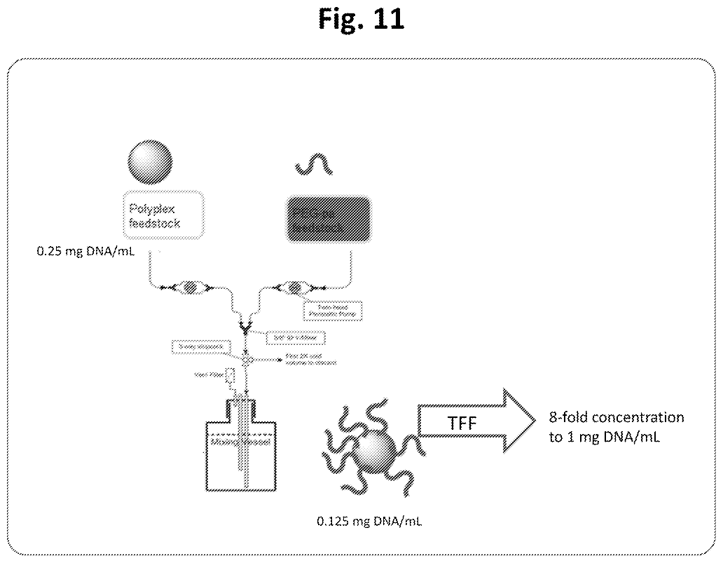

DD-X—DNA (pVax-PD-L1-Fc) polyplexes were produced at N:P ratio of 7:1 and DNA concentration of 0.25 mg/mL DNA with 5% trehalose as previously described. PEG-PGA solution in 5% trehalose was then mixed with DD-X-DNA polyplex at a 1:1 volume ratio by in line mixing (each fluid stream at 7 mL/min) to yield PEGylated polyplex at 0.125 mg/mL DNA and N:P:A ratio of 7:1:17.5. After incubating at 60 minutes at room temperature, the PEGylated polyplex was concentrated by TFF (regenerated cellulose membrane, MWCO 10 kDa, 15 psig inlet pressure) using the process previously described in U.S. Pat. No. 8,722,646B2. During TFF, aliquots were taken at nominal DNA concentrations of 0.25 mg/mL, 0.5 mg/mL. Final product collected up to a nominal DNA concentration of about 1 mg/mL. Samples were aliquoted and stored at −80° C. After thawing, samples were tested for particle size, PDI, zeta potential, free DNA and % supercoil DNA. A schematic is provided in FIG. 11.

| Sample ID | [DNA] | Comment |
|---|---|---|
| CMC-INT06-099 A | 0.25 mg/mL | DD-X-DNA polyplex, |
| | | N:P 7:1 |
| CMC-INT06-099 B | 0.125 mg/mL | PEGylated polyplex, |
| | | N:PA 7:1:17.5, pre-TFF |
| CMC-INT06-099 C | 1 mg/mL | PEGylated polyplex, |
| | | N:PA 7:1:17.5, post-TFF |

7.2 Results

The particle size, PDI, zeta potential, free DNA and % supercoil of the thawed test samples were found to be:

| | | | Ave. | | |
|---|---|---|---|---|---|
| | Ave. | | Zeta | | |
| | Size | Ave. | Potential | | Supercoil |
| Sample ID | (nm) | PDI | (mV) | Free DNA | Content (%) |
| CMC-INT06-099 A | 121.9 | 0.171 | 42.8 | Non-visible | 91.5 |
| CMC-INT06-099 B | 129.7 | 0.129 | −5.1 | Non-visible | 91.2 |
| In-process TFF | 130.9 | 0.138 | −5.2 | Not | Not |
| (0.25 mg/mL) | | | | determined | determined |
| In-process TFF | 133.4 | 0.143 | −4.1 | Not | Not |
| (0.5 mg/mL) | | | | determined | determined |
| CMC-INT06-099 C | 135.0 | 0.146 | −3.9 | Non-visible | 90.1 |

7.3 Conclusion

PEGylated polyplex was able to be concentrated from 0.125 mg/mL DNA to 1 mg/mL DNA by TFF concentration process, and maintain colloidally stable nanoparticles.

### 8. Concentrated PEGylated Polyplex by PEGylation of Pre-Concentrated Polyplexes

8.1 Procedure

Concentrated DD-X—DNA (pVax-PD-L1-Fc) polyplexes were produced at N:P ratio of 7:1 and DNA concentration of 1 mg/mL DNA with 9% sucrose as previously described in U.S. Pat. No. 8,722,646B2. PEG-PGA solution in water was then mixed with DD-X-DNA polyplex at a 1:1 volume ratio by in line mixing (each fluid stream at 7 mL/min) to yield PEGylated polyplex at 0.5 mg/mL DNA and N:P:A ratio of 7:1:7 or N:P:A ratio of 7:1:17.5. After incubating 60 minutes at room temperature, the PEGylated polyplexes were aliquoted and stored at −80° C.

After thawing, samples were tested for particle size, PDI, zeta potential, free DNA and % supercoil DNA.

| | Nominal | |
|---|---|---|
| Sample ID | [DNA] | Comments/Description |
| 309-26 | 1 mg/mL | Non-PEGylated polyplex, 9% sucrose |
| CMC-INT06- | 0.5 mg/mL | PEGylated polyplex, N:P:A = |
| 124 A | | 7:1:17.5, 4.5% sucrose |
| CMC-INT06- | 0.5 mg/mL | PEGylated polyplex, N:P:A = |
| 124 B | | 7:1:7, 4.5% sucrose |

8.2 Results

### Physicochemical Properties of Source Polyplex and PEGylated Polyplexes

| | | Particle | | Zeta | | | Supercoil |
|---|---|---|---|---|---|---|---|
| | | Size | | Potential | | Free | Content |
| Entity | Appearance | (nm) | PDI | (mV) | pH | DNA | (%) |
| 309-26 | Clear/ | 129.4 | 0.146 | 34.3 | Not | Non- | 86.4 |
| | translucent | | | | determined | visible | |
| CMC- | Clear/ | 141.0 | 0.116 | −2.46 | 7.1 | Non- | 86.4 |
| INT06-124 A | translucent | | | | | visible | |
| CMC- | Clear/ | 141.2 | 0.105 | 1.93 | 6.59 | Non- | 87.1 |
| INT06-124 B | translucent | | | | | visible | |

8.3 Conclusion

DD-X—DNA polyplex at 1 mg/mL DNA was PEGylated at N:P:A ratios of 7:1:7 and 7:1:17.5 to final DNA concentration of 0.5 mg/mL with no visible aggregation.

9. PEGylation of Polyplexes with PEG-PGA or PEG-PAA

9.1 Procedure

N:P 7 polyplex (CMC-INT06-24C) aliquots were PEGylated using PEG-PGA or PEG-PAA as described herein. The resulting PEGylated polyplexes were tested for physicochemical properties and DNA capture. The resulting formulations, in addition to the N:P 7 polyplex, were generated.

| | | NPA | |
|---|---|---|---|
| Sample name | | ratio | Comments |
| CMC-INT06- | NP7 | 7:1:0 | DD-X-DNA (pVax-opt- |
| 24C | | | hIL10) polyplex, 5% |
| | | | trehalose, 0.25 mg/mL |
| CMC-INT06- | PEG5kPGA_A | 7:1:1.75 | PEGylated with PEG-PGA |
| 059 | PEG5kPGA_B | 7:1:2.45 | (mPEG5K-b-PLE10), 5% |
| | PEG5kPGA_C | 7:1:3.22 | trehalose, 0.125 mg/mL |
| CMC-INT06- | PEG5kPAA_D | 7:1:1.75 | PEGylated with PEG-PAA |
| 059 | | | (mPEG5K-b-PLD10), 5% |
| CMC-INT06- | PEG5kPAA_E | 7:1:2.45 | trehalose, 0.125 mg/mL |
| 059 | | | |
| CMC-INT06- | PEG5kPAA_F | 7:1:3.22 | |
| 059 | | | |

9.2 Results

| | | | | | Zeta |
|---|---|---|---|---|---|
| | | NPA | Diameter | | potential |
| Sample name | | ratio | (nm) | PDI | (mV) |
| CMC- | NP7 | 7:1:0 | 138 ± 1.65 | 0.20 ± 0.01 | 30 ± 0.76 |
| INT06- | | | | | |
| 24C | | | | | |
| CMC- | PEG5kPGA_A | 7:1:1.75 | 168 ± 3.2 | 0.16 ± 0.02 | −2.8 ± 0.23 |
| INT06- | PEG5kPGA_B | 7:1:2.45 | 160 ± 0.82 | 0.18 ± 0.00 | −3.7 ± 0.42 |
| 059 | PEG5kPGA_C | 7:1:3.22 | 155 ± 0.26 | 0.18 ± 0.00 | −3.8 ± 0.27 |
| CMC- | NP7 | 7:1:0 | 137 ± 0.72 | 0.18 ± 0.01 | 33 ± 0.22 |
| INT06- | | | | | |
| 24C | | | | | |
| CMC- | PEG5kPAA_D | 7:1:1.75 | 174 ± 0.09 | 0.18 ± 0.0 | −3.5 ± 0.73 |
| INT06- | PEG5kPAA_E | 7:1:2.45 | 161 ± 0.42 | 0.18 ± 0.00 | −4.4 ± 0 |
| 059 | PEG5kPAA_F | 7:1:3.22 | 156 ± 0.44 | 0.18 ± 0.00 | −2.9 ± 0.28 |

Samples A-F were tested for free DNA as described herein. The resulting gel showed no visible free DNA.

9.3 Conclusion

PEGylated polyplexes with either PEG-PGA (mPEG5K-b-PLE10) or PEG-PAA (mPEG5K-b-PLD10) formed stable nanoparticles with no free DNA.

### 10. Stability of PEGylated Polyplexes in Simulated Intestinal Fluid

10.1 Procedure

### Test Articles

N:P 7 polyplex (CMC-INT06-24C) aliquots were PEGylated using PEG-PGA as described herein. The resulting PEGylated polyplexes were tested for stability in FaSSIF buffers.

| | | NPA | |
|---|---|---|---|
| Sample name | | ratio | Comments |
| CMC- | NP7 | 7:1:0 | DD-X-DNA (pVax-opt- |
| INT06- | | | hIL10) polyplex, 5% |
| 24C | | | trehalose, 0.25 mg/mL |
| CMC- | PEG5kPGA_A | 7:1:1.75 | PEGylated with PEG-PGA |
| INT06- | PEG5kPGA_B | 7:1:2.45 | (mPEG5K-b-PLE10), 5% |
| 059 | PEG5kPGA_C | 7:1:3.22 | trehalose, 0.125 mg/mL |

### Testing Stability of PEGylated Polyplexes in SIF 1:3, v/v

Add 50 uL of polyplexes to 150 uL of simulated intestinal fluid. Mix well. Immediately aliquot 50 uL of the suspension into a DLS cuvette and add 350 uL of water and measure particle size by DLS as described. Split the remaining sample solution into three tubes. At the appropriate timepoints, add 350 uL of water to the tubes, mix thoroughly, and measure particle size by DLS.

### Testing Stability of PEGylated Polyplexes in SIF 3:1, v/v

Add 75 uL of polyplex to 25 uL of simulated intestinal fluid. Mix well. Immediately aliquot 50 uL of the suspension into a DLS cuvette and add 350 uL of Water and measure particle size by DLS as described. Split the remaining sample solution into three tubes. At the appropriate timepoint, add 350 uL of Water to the tubes, mix thoroughly and measure particle size by DLS.

10.2 Results

Non-PEGylated polyplexes (represented as 7-1-0) aggregated immediately on mixing with the buffer (maximum on scale). PEGylated polyplexes (with PEG-PGA, mPEG5K-b-PLE10) remained stable over 24 h at 1:3 (FaSSIF:PEGylated polyplex, v/v) ratio. PEGylated polyplexes (with PEG-PGA, mPEG5K-b-PLE10) remained stable over 1 h at 3:1 (FaSSIF:PEGylated polyplex, v/v) ratio.

### 11. Stability of PEGylated Polyplexes in FaSSIF Buffer-Free DNA

### Reference: CMC-INT06-EXP-072

11.1 Procedure

N:P 7 polyplex (CMC-INT06-024C, DD-X—DNA (pVax-opt-hIL10) polyplex, 5% trehalose, 0.25 mg/mL) aliquots were PEGylated using PEG-PGA (in 5% trehalose) as described herein to attain the target N:P:A ratios in the following table. The resulting PEGylated polyplexes were tested for stability in FaSSIF buffers according to the ratio in the following table. After 2 h, the following physicochemical properties were determined: free DNA, pH, diameter and PDI.

| | | | Mixing | | |
|---|---|---|---|---|---|
| | | | volume | DNA | |
| | | | (FaSSIF | Concen- | |
| | N:P:A | | uL + | tration | |
| Sample name | ratio | Buffer | PP uL) | (ug/mL | pH |
| INT06-EXP-072-A | 7:1:0 | FaSSIFV1 | 75 + 50 | 50 | 6.71 |
| INT06-EXP-072-B | 7:1:1.75 | FaSSIFV1 | 75 + 50 | 50 | 6.00 |
| INT06-EXP-072-C | 7:1:2.45 | FaSSIFV1 | 75 + 50 | 50 | 6.04 |
| INT06-EXP-072-D | 7:1:3.22 | FaSSIFV1 | 75 + 50 | 50 | 6.27 |
| INT06-EXP-072-E | 7:1:0 | FaSSIFV2 | 75 + 50 | 50 | 6.71 |
| INT06-EXP-072-F | 7:1:1.75 | FaSSIFV2 | 75 + 50 | 50 | 5.93 |
| INT06-EXP-072-G | 7:1:2.45 | FaSSIFV2 | 75 + 50 | 50 | 5.98 |
| INT06-EXP-072-H | 7:1:3.22 | FaSSIFV2 | 75 + 50 | 50 | 6.14 |
| INT06-EXP-072-I | 7:1:0 | FaSSIFV1 | 50 + 100 | 83.3 | 6.70 |
| INT06-EXP-072-J | 7:1:1.75 | FaSSIFV1 | 50 + 100 | 83.3 | 6.70 |
| INT06-EXP-072-K | 7:1:2.45 | FaSSIFV1 | 50 + 100 | 83.3 | 5.63 |
| INT06-EXP-072-L | 7:1:3.22 | FaSSIFV1 | 50 + 100 | 83.3 | 5.74 |
| INT06-EXP-072-M | 7:1:0 | FaSSIFV2 | 50 + 100 | 83.3 | 6.63 |
| INT06-EXP-072-N | 7:1:1.75 | FaSSIFV2 | 50 + 100 | 83.3 | 5.66 |
| INT06-EXP-072-O | 7:1:2.45 | FaSSIFV2 | 50 + 100 | 83.3 | 5.67 |
| INT06-EXP-072-P | 7:1:3.22 | FaSSIFV2 | 50 + 100 | 83.3 | 5.72 |

11.2 Results

| | | Diameter | | Zeta potential | |
|---|---|---|---|---|---|
| Polyplex lot | N:P:A ratio | (nm) | PDI | (mV) | PH |
| CMC-INT06-024 C | 7:1:0 | 141 ± 1.98 | 0.18 ± 0.01 | 33 ± 0.22 | |
| CMC-INT06-072 A | 7:1:1.75 | 178.56 ± 2.11 | 0.175 ± 0.014 | −2.5 ± 0.28 | 5.60 |
| CMC-INT06-072 B | 7:1:2.45 | 197.9 ± 1.44 | 0.181 ± 0.015 | −1.9 ± 0.13 | 5.67 |
| CMC-INT06-072 C | 7:1:3.22 | 213.43 ± 0.737 | 0.19 ± 0.00 | −2.2 ± 0.39 | 5.59 |

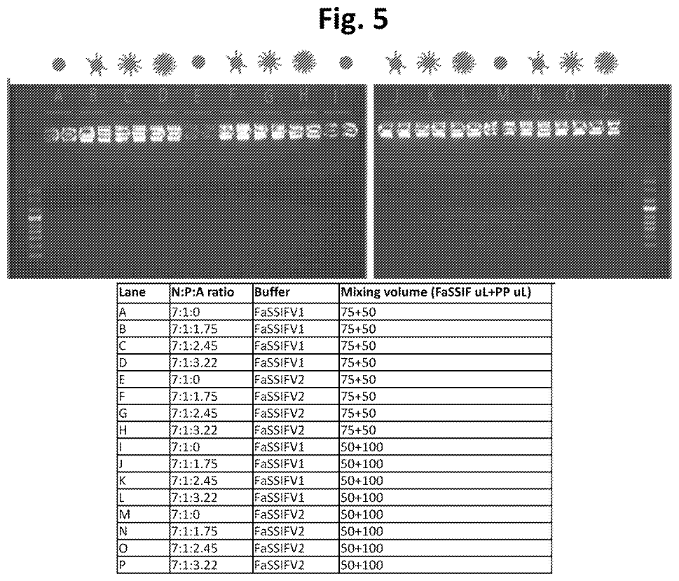

Free DNA assay of FaSSIF-Polyplex samples. No free DNA was observed as shown in FIG. 5.
Lane descriptions are provided below.

| | Sample Description | | | |
|---|---|---|---|---|
| | | | | Mixing volume |
| | | | | (FaSSIF uL + |
| Lane | Sample name | N:P:A ratio | Buffer | PP uL) |
| A | INT06-EXP-072-A | 7:1:0 | FaSSIFV1 | 75 + 50 |
| B | INT06-EXP-072-B | 7:1:1.75 | FaSSIFV1 | 75 + 50 |
| C | INT06-EXP-072-C | 7:1:2.45 | FaSSIFV1 | 75 + 50 |
| D | INT06-EXP-072-D | 7:1:3.22 | FaSSIFV1 | 75 + 50 |
| E | INT06-EXP-072-E | 7:1:0 | FaSSIFV2 | 75 + 50 |
| F | INT06-EXP-072-F | 7:1:1.75 | FaSSIFV2 | 75 + 50 |
| G | INT06-EXP-072-G | 7:1:2.45 | FaSSIFV2 | 75 + 50 |
| H | INT06-EXP-072-H | 7:1:3.22 | FaSSIFV2 | 75 + 50 |
| I | INT06-EXP-072-I | 7:1:0 | FaSSIFV1 | 50 + 100 |
| J | INT06-EXP-072-J | 7:1:1.75 | FaSSIFV1 | 50 + 100 |
| K | INT06-EXP-072-K | 7:1:2.45 | FaSSIFV1 | 50 + 100 |
| L | INT06-EXP-072-L | 7:1:3.22 | FaSSIFV1 | 50 + 100 |
| M | INT06-EXP-072-M | 7:1:0 | FaSSIFV2 | 50 + 100 |
| N | INT06-EXP-072-N | 7:1:1.75 | FaSSIFV2 | 50 + 100 |
| O | INT06-EXP-072-O | 7:1:2.45 | FaSSIFV2 | 50 + 100 |
| P | INT06-EXP-072-P | 7:1:3.22 | FaSSIFV2 | 50 + 100 |

### 12. Preparation of PEGylated Polyplexes and In Vitro Transfection

12.1 In Vitro Transfection Reagents

| Material | Cat. No. |
|---|---|
| Multicell sterile water | 809-115-CL |
| Dulbecco's Modified Eagle Medium (DMEM), | 319-005-CL |
| high glucose 1X (Mod.) | |
| Fetal bovine serum (FBS) | 26140-079 |
| Penicillin/streptomycin (10,000 U/mL) | 450-201-EL |
| Opti-MEM media | 31985-070 |
| Phosphate buffered saline (PBS) | 311-425-CL |
| cOmplete ™ Protease Inhibitor Cocktail | 11697498001 |

12.2 In Vitro Transfection Consumables

| Material | Cat. No. |
|---|---|
| Poly-L-lysine 96-well clear bottom TC plates | 356516 |
| 96-well PP, round bottom | 3365 |
| 15 mL tube | 430790 |
| 50 mL tube | 430828 |
| Axygen MaxyClear Snaplock Microtubes, 1.5 mL | 14222155 |

### In Vitro Transfection Equipment

| Description | Manufacturer |
|---|---|
| Centrifuge, Allegra 6R | Beckman |
| 12-channel pipette, P 50-1200 | Biohit Sartorius |
| 12-channel pipette, P 30-300 | Biohit Sartorius |
| 12-channel pipette, P 10-100 | Biohit Sartorius |
| Vi-Cell Auto Cell Viability Analyzer | Beckman |
| Incubator, model 3110 | ThermoFisher |
| Centrifuge, model 5415D | Eppendorf |
| MSD plate reader | Meso Scale Diagnostics, Ltd. |
| Cirascan ™ Imager | Quanterix/Aushon |
| Titer plate shaker, model 00122 | Labline Instruments |

12.4 Procedure

### Test Articles

N:P 7 polyplex (CMC-INT06-024C, DD-X—DNA (pVax-opt-hIL10) polyplex, 5% trehalose, 0.25 mg/mL) aliquots were PEGylated using PEG-PGA (mPEG5K-b-PLE10, in 5% trehalose) as described herein to attain the target N:P:A ratios in the following table. The resulting PEGylated polyplexes were tested for particle diameter, PDI, zeta potential before and after freeze/thaw. Thawed samples were tested for in vitro transfection.

| Sample ID | N:P:A | Comments |
|---|---|---|
| INT06-024C | 7:1:0 | DD-X-DNA (pVax-opt-hIL10) polyplex, |
| | | 5% trehalose, 0.25 mg/mL |
| INT06-EXP-084 A | 7:1:3.2 | PEGylated with PEG-PGA (mPEG5K-b- |
| INT06-EXP-084 B | 7:1:2.5 | PLE10), 5% trehalose |
| INT06-EXP-084 C | 7:1:1.75 | |

12.5 In Vitro Transfection Procedure

### Cell Preparation

HEK293T cells (ATCC) at passage 19 were passed and centrifuged to remove trypsin, then resuspended 2-fold in DMEM supplemented with 10% FBS. Cell viability and count was performed in a Vi-Cell Auto Cell Viability Analyzer to verify cell count and viability. Cells were diluted to a concentration of 1.25×10⁵ cells/mL in DMEM supplemented with 10% FBS and dispensed into 96-well, clear bottom TC plates (200 uL, 25,000 cells per well). The plates were incubated at room temperature for 15-20 minutes before incubating overnight at 37° C./5% CO2 and then used for transfection.

### Dilution of Polyplex

Test samples were diluted to 0.125 mg/mL DNA, unless otherwise provided at the target concentration. Control samples (CMC-INT06-024 and CMC-JAN01-010) provided at 0.25 mg/mL DNA diluted in water to 0.14 mg/mL. All test and control samples were then serially diluted in water, 1/1.35 for a total of 10 dilutions. These serial dilutions were further diluted 17.7 fold in Opti-MEM and 35.6 uL of this diluted polyplex was added to each well in duplicate (giving a final range of doses from 282 ng of polyplex to 19 ng of polyplex for the control samples and 251 ng of polyplex to 17 ng of polyplex for the test samples).

### Transfection of Cells

Transfection was carried out as follows. First, media was removed from each HEK293T well followed by addition of 0.1 mL Opti-MEM (pH 7.4) and then removal. Polyplex samples diluted in Opti-MEM (see previous section) were added to each well and incubated at 37° C. for 3 hours. After incubation, the media was removed and replaced with 0.2 mL of complete media and re-incubated at 37° C. After 48 hours the supernatant was removed and used immediately for the MSD assay. The remaining cells was lysed to determine total protein.

### Quantification of Total Protein

Total protein for each well was determined using the DC™ Protein assay using BSA for the standard curve. Once the supernatant was removed for the ELISA the remaining cell layer was lysed in lysis buffer for 10 minutes at 4° C. Lysates were pipetted several times (while minimizing bubble formation) before transferring to a v-bottom, 96-well plate. The lysates were then clarified by centrifuging for 5 minutes at 4° C. at 1000 g

### Preparing Protein Standard Curve

Protein Assay Standard II (Bovine serum albumin) stock was prepared at 5 mg/ml in milli-Q water and stored at 4° C. The standard curve was prepared by performing 1:2 serial dilutions of the protein standard in lysis buffer (the same buffer in which the samples are prepared).

Lowry/DCTM protein Assay

Working reagent A′ was prepared by adding 20 μl of reagent S to each 1 ml of reagent A. 25 μl of Working reagent A′ was transferred per well of the 96-well plates. 5 μl of cell lysate or standard was added per well. 200 μl of reagent B was then added to each well. The plates were shaken for 5 seconds and incubated for 15 minutes at room temperature to allow colour to develop. Absorbance was measured at 750 nm on the SpectraMAX plate reader. The absorbance of the standards was plotted against the standard concentration. The SpectraMax software curve fitting analysis was used and the four parameter algorithm provided the best curve fit for the standard curve. The software also interpolated the protein concentration of the samples from the protein standard curve.

Quantification of hIL-10 Protein by MSD Assay

Supernatants were centrifuged at 1000×g for 10 minutes and then diluted appropriately. All reagents for the assay were equilibrated to room temperature before use. Standard for the assay were prepared by reconstituting hIL-10 in the Diluent 2 to the specified concentration, incubated for 15 minutes at room temperature, and then serially-diluted 4-fold with Diluent 2 to prepare the 7 doses of the standard curve. Add 50 μL of standard in duplicate to 2 columns of the MSD plates. Add 25 μL of Diluent 2 and 25 μL of sample to the remaining wells. Cover the plates and incubate at room temperature with shaking at 300 rpm for 2 hours. Wash the plates and then add 25 μL of diluted Detection antibody. Incubate at room temperature with shaking at 300 rpm for 2 hours. Wash the plates and then add 150 μL 2× Read Buffer T. Read using the Mesoscale using the barcode (VPLEX single spot is automatically assigned). The absorbance of the standards was plotted against the standard concentration and the amount of hIL-10 of the samples was interpolated using Prism GraphPad software with the four-parameter algorithm. Once the amount of hIL-10 was determined in each well it was normalized to the total protein determined from the DC™ Protein assay (ie. ng hIL-10 per mg of total protein). The dose response curves were prepared in GraphPad Prism by plotting the ng hIL-10 per mg of total protein versus the amount of transfected DNA (ng). EC50 and the predicted maximum amount of hIL-10 per mg of total protein was determined.

12.6 Results

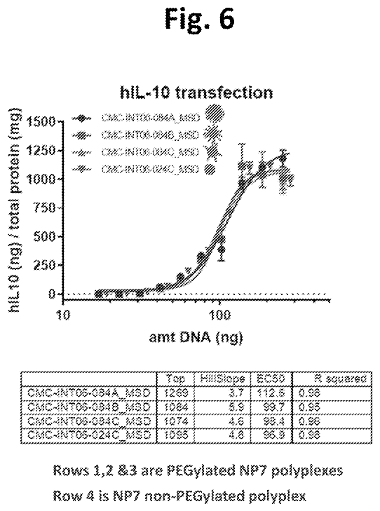

All particles were stable with no aggregation. Zeta potential reduced to −3 mV for all samples after PEGylation. After freeze thaw polyplexes showed same properties. In vitro transfection results were similar for non-PEGylated and PEGylated samples (FIG. 6).

| | | | | |
|---|---|---|---|---|
| | | | | Zeta |
| | N:P:A | Diameter | | potential |
| Sample name | ratio | (nm) | PDI | (mV) |

Fresh

| | | | | |
|---|---|---|---|---|
| INT06-EXP-084 A | 7:1:3.2 | 153.17 ± 1.46 | 0.18 ± 0.01 | −3.01 ± 0.07 |
| INT06-EXP-084 B | 7:1:2.5 | 159.93 ± 1.85 | 0.19 ± 0.02 | −3.23 ± 0.62 |
| INT06-EXP-084 C | 7:1:1.75 | 159.87 ± 0.35 | 0.19 ± 0.01 | −3.04 ± 0.17 |

After Freeze/Thaw

| | | | | |
|---|---|---|---|---|
| INT06-EXP-084 A | 7:1:3.2 | 152.33 ± 1.52 | 0.17 ± 0.01 | −3.28 ± 0.37 |
| INT06-EXP-084 B | 7:1:2.5 | 159.67 ± 1.52 | 0.17 ± 0.02 | −3.11 ± 0.21 |
| INT06-EXP-084 C | 7:1:1.75 | 161.13 ± 0.75 | 0.16 ± 0.01 | −3.18 ± 0.53 |

### 13. PEGylation of Polystyrene Beads

### Reference: CMC-INT06-EXP-039 and CMC-INT06-EXP-055

13.1 Procedure

### PEGylation of Orange Polystyrene Beads

Generally: Transfer 1 mL of amine-modified polystyrene beads to a 7 mL glass bottle with a magnetic stir bar. Add 5 uL of 1 M HCl. In a 1.5 mL eppendorf tube dissolve 70 mg of PEG NHS and 8 mg of EDC-HCl in 1 mL of water until it forms a clear solution. Slowly add 0.7 mL of this solution to the suspension of polystyrene. Stir the solution at room temperature for overnight while protecting from light. PEGylated orange particles were purified and concentrated as described below. Particle sizing and zeta potential measurement was used to confirm PEGylation of the PS particles.

### PEGylation of Green Polystyrene Beads

Generally: Transfer 2 mL of green amine-modified polystyrene beads to a 7 mL glass bottle with a magnetic stir bar. Add 10 uL of 1 M HCl. In a 1.5 mL eppendorf tube dissolve 100 mg of PEG-NHS and 5 mg of EDC-HCl in 1 mL water until it forms a clear solution. Slowly add 0.9 mL of this solution to the suspension of polystyrene. Stir the solution at room temperature for overnight while protecting from light. PEGylated green particles were purified and concentrated as described below. Particle sizing and zeta potential measurement was used to confirm PEGylation of the PS particles.

### PEGylation of Blue Polystyrene Beads

Generally: Transfer 1 mL of blue amine-modified polystyrene beads to a 7 mL glass bottle with a magnetic stir bar. Add 2 mL water. In a 1.5 mL eppendorf tube dissolve 270 mg of PEG-NHS and 10.34 mg of EDC-HCl in 1 mL water until it forms a clear solution. Slowly add 0.9 mL of this solution to the suspension of polystyrene. Stir the solution at room temperature for overnight while protecting from light. PEGylated blue particles were purified and concentrated as described below. Particle sizing and zeta potential measurement was used to confirm PEGylation of the PS particles.

### Purification and Concentration of PEGylated Polystyrene

PEGylated PS particles were purified and concentrated as follows. Aliquot 300 uL of the reaction mixture to an Amicon Ultra filter. Centrifuge at 5000 rpm for 10 minutes. Remove the liquid from the bottom and add 300 uL of water to the top of the filter (the beads are retained on the filter). Repeat centrifugation and washing four times to wash the beads. Pool the beads from different filters onto a single filter. Add 300 uL of water and centrifuge again. The green beads and orange beads were suspended in water up to about 1.5 mL.

Measure hydrodynamic diameter of polystyrene particles by mixing 10 uL of the suspension in 390 uL of water. Measure zeta potential of polystyrene particles by mixing 20 uL of the particle suspension in 780 uL of 10 mM NaCl.

13.2 Results

At the end of the reaction if the zeta potential decreased considerably which demonstrated that PEGylation occurred.

| | Zeta | Zeta | | | |
|---|---|---|---|---|---|
| | potential | potential | | | |
| | before | after | Diameter of | PDI of | Final |
| | PEGylation | PEGylation | PEGylated | PEGylated | concentration |
| Sample ID | (mV) | (mV) | beads (nm) | beads | (wt %) |
| INT06-039- | 62 | +9 ± 0.76 | 127 ± 1.81 | 0.19 ± 0 | 5 |
| Orange beads | | | | | |
| INT06-039- | 48 | 8 ± 0.53 | 325 ± 3.15 | 0.19 ± 0.03 | 3 |
| Green beads | | | | | |
| INT06-055-A | 62 | 4 ± 1 | 136 ± 6 | 0.18 ± 0.02 | 1.25 |
| and INT06-055- | | | | | |
| B (orange) | | | | | |
| INT06-055-C | 62 | 9.0 ± 0.2 | 71.11 ± 0.08 | 0.10 ± 0.01 | 2.5 |
| and INT06-055- | | | | | |
| D (blue) | | | | | |

### 14. Preparation of RITC-DDX+DNA Polyplex and PEGylated RITC-DD-X+DNA Polyplex

References: provided below

14.1 RITC-DD-X Reagents

| Material | Supplier | Cat. No. |
|---|---|---|
| Rhodamine B isothiocyanate (RITC), | Sigma-Aldrich | 283924-100MG |
| MW = 536.08 | | |
| Methanol, anhydrous, 99.9% | VWR | AA41467-AK |
| DMSO | Sigma-Aldrich | 34943-1L |
| D2O | Aldrich | 151882-100G |
| DC1/D2O (35 wt %) | Aldrich | 543047 |
| Methanol | Fisher chemicals | A454-4 |
| Isopropyl alcohol | Fisher | A464-4 |
| Nitrogen gas | Praxair | |
| Dialysis tubing, Spectra/Por 6 | Spectrum Labs | 132638 |
| Prewetted, 1 kD | | |
| Buchner filter, PES 0.2 um | Corning | 431118 |

14.2 RITC-DD-X Equipment

| Description | Manufacturer |
|---|---|
| NMR, Avance II 500 MHz, | Bruker |
| Probe BBI | |

14.3 Procedure

### RITC-Labelled DD-X

RITC was conjugated to DD-X based on the method derived from Carbohydrate Polymers 72 (2008) 616-624. Briefly, DD-X stock (lot JAN03-009) was dissolved in water to attain concentration of 40 mM and pH 5.8. The DD-X solution was mixed with an equal volume of DMSO and stirred at room temperature for 1 hour, then bubbled with nitrogen gas for 15 min. In a separate container, RITC was dissolved in DMSO (target 3.7 mg/mL) and added dropwise to the DD-X/DMSO mixture (RITC/DMSO:DD-X/DMSO 1:8, v/v). The resulting mixture was stirred for 65 hours at room temperature protected from light. The RITC-labelled DD-X was purified by dialysis against water and then freeze-dried. Unreacted RITC was extracted with methanol and washed on a Buchner funnel with methanol until the filtrate was colorless. The washed powder was collected and vacuum dried overnight at room temperature. Final collected powder was pink in appearance. Conjugation of RITC to DD-X was confirmed by H-NMR spectroscopy: RITC-DD-X was dissolved (8 mg/mL) in D20+DC1/D20 to attain pH around 2.5.

### RITC-DD-X+DNA Polyplex

A 50/50 blend of RITC-DD-X (described above) and unlabeled DD-X was prepared and then used to make an N:P 20 polyplex (0.1 mg/mL DNA) in 5% trehalose with GWiz-GFP plasmid DNA according to drip method procedure described herein.

### RITC-DD-X+DNA Polyplex

A 33/67 blend of RITC-DD-X (described above) and unlabeled DD-X was prepared and then used to make an N:P 7 polyplex (0.25 mg/mL DNA) in 5% trehalose with pVax-opt-hIL10 plasmid DNA according to in-line mixing procedure described herein.

### PEGylated RITC-DD-X+DNA Polyplex

N:P 7 polyplex aliquots were PEGylated using PEG-PGA as described herein. The resulting PEGylated polyplexes were tested for particle size, PDI and zeta potential. The resulting formulations in addition to the N:P 7 polyplex, were generated.

| | NPA | |
|---|---|---|
| Sample name | ratio | Comments |
| CMC-INT06-070 | 7:1:0 | RITC-DD-X + DNA (pVax-opt-hIL10) |
| | | polyplex, 5% trehalose, 0.25 mg/mL |
| CMC-INT06-075 A | 7:1:1.75 | PEGylated with PEG-PGA (mPEG5K- |
| | | b-PLE10), 5% trehalose |
| CMC-INT06-075 B | 7:1:2.5 | |
| CMC-INT06-075 C | 7:1:3.2 | |
| CMC-INT06-075 D | 7:1:1.75 | PEGylated with PEG-PGA (mPEG5K- |
| CMC-INT06-075 E | 7:1:2.5 | b-PLE50), 5% trehalose |
| CMC-INT06-075 F | 7:1:3.2 | |
| CMC-INT06-075 G | 7:1:1.75 | PEGylated with PEG-PGA (mPEG5K- |
| CMC-INT06-075 H | 7:1:2.5 | b-PLE10), 5% trehalose |
| CMC-INT06-075 I | 7:1:3.2 | |

14.4 Results

Results of particle size, PDI and zeta potential

| | | | | Zeta potential |
|---|---|---|---|---|
| Sample name | N:P:A Ratio | Diameter (nm) | PDI | (mV) |
| CMC-INT06- | 7:1:0 | 125.2 ± 0.3 | 0.17 ± 0.01 | 32.23 ± 1.44 |
| 070 | | | | |
| CMC-INT06- | 7:1:1.75 | 144.93 ± 1.19 | 0.19 ± 0.01 | −3.04 ± 0.43 |
| 075 A | | | | |
| CMC-INT06- | 7:1:2.5 | 142.67 ± 2.5 | 0.17 ± 0 | −3.06 ± 0.36 |
| 075 B | | | | |
| CMC-INT06- | 7:1:3.2 | 150.04 ± 0.7 | 0.17 ± 0.02 | −3.33 ± 0.1 |
| 075 C | | | | |
| CMC-INT06- | 7:1:1.75 | Visible | Visible | Visible |
| 075 D | | aggregation | aggregation | aggregation |
| CMC-INT06- | 7:1:2.5 | Visible | Visible | Visible |
| 075 E | | aggregation | aggregation | aggregation |
| CMC-INT06- | 7:1:3.2 | Visible | Visible | Visible |
| 075-F | | aggregation | aggregation | aggregation |
| CMC-INT06- | 7:1:1.75 | 142.10 ± 1.04 | 0.17 ± 0.0 | −3.01 ± 0.55 |
| 075 G | | | | |
| CMC-INT06- | 7:1:2.5 | 146.5 ± 1.21 | 0.17 ± 0.02 | −2.73 ± 0.58 |
| 075 H | | | | |
| CMC-INT06- | 7:1:3.2 | 149.77 ± 1.83 | 0.17 ± 0.01 | −3.14 ± 0.68 |
| 075 I | | | | |

Polyplexes CMC-INT06-075 D, E and F were aggregated.

15. Evaluation of the Aggregation of Different PS Beads Inside Type III Mucin and Quantification of their Diffusion Through 1 and 3 μm Transwells

Reference: CMC-INT06-EXP-042

15.1 Mucin diffusion materials

| Material | Cat. No. | Comments |
|---|---|---|
| Amine-modified PS beads, blue, | L0780 | Blue (Ex: 360, |
| 50 nm, 2.5% w/w | | Em: 420) |
| Orange PEG-PS beads | INT06-039-Orange | Orange (Ex: 481, |
| | beads | Em: 644) |
| 24 well suspension culture plate | 662102 | |
| (hydrophobic) | | |
| Mucin from porcine stomach, | M1778 | |
| type 3 | | |
| HTS multiwell insert system with | 351183 | |
| 3 um PET membrane | | |
| RITC-DD-X + DNA Polyplex | CMC-INT05-026 | |
| | D | |
| 96W plate, black, flat bottom | 3915 | |
| μ-Slide VI 0.1 | 80666 | |

15.2 Mucus diffusion Equipment

| Description | Manufacturer |
|---|---|
| Plate reader, EnVision 2105 | Perkin Elmer |
| Analytical Balance | Mettler Toledo |
| Incubator, model 3110 | ThermoFisher |
| Microscope, EVOS FL Auto | Thermofisher |

15.3 Procedure

### Preparation of Type III Mucin Suspension

Type III mucin suspension was prepared by dispensing Type III mucin in water to a target concentration from 5% w/w to 0.1% w/w, as required.

### Aggregation in Mucin Test

Test samples (RITC-labelled polyplex, amine modified PS beads, or PEGylated PS beads) were combined with Type III mucin suspensions in microtubes at various concentrations to attain final target concentrations of the test sample and mucin. The sample-mucin samples were protected from light and mixed at 30 rpm at room temperature. At 30 and 60 minutes, the mixtures were aliquoted to an Ibidi u-slide and observed under an EVOS microscope for visible aggregation.

### Evaluation of Diffusion Through Mucin-Loaded Transwells

### Sample Preparation.

Test samples (RITC-labelled polyplex, amine modified PS beads, or PEGylated PS beads) were combined with Type III mucin suspensions in microtubes to attain final target concentrations of the test sample and final mucin concentration of 0.4%. Samples were protected from light and mixed at 40 rpm for 1 hour prior to loading in transwell insert.

### Transwell Preparation and Sample Diffusion

Transwell bottoms were filled 0.6 mL of 0.4% Type III mucin. HTS transwell inserts (with 1 um or 3 um PET membranes) were loaded with 0.1 mL of pre-mixed sample+mucin. Controls were loaded without 0.4% mucin only. Reference (to mimic 100% diffusion) was pre-mixed sample+mucin without a transwell insert (0.7 mL). After 20 hours incubation at 37° C., the diffusion of test samples into the bottom wells was evaluated by fluorescence plate reader (Envision).

15.4 Results

### Aggregation in Mucin Test

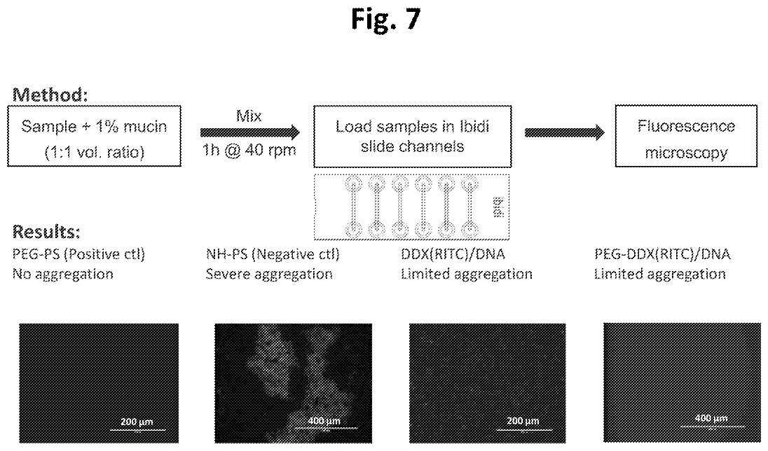

As shown in FIG. 7, both RITC-labelled polyplex and amine modified PS beads showed aggregation in mucin after incubation for 1 hour. PEGylated PS beads did not have visible aggregation.

### Evaluation of Diffusion Through Mucin-Loaded Transwells

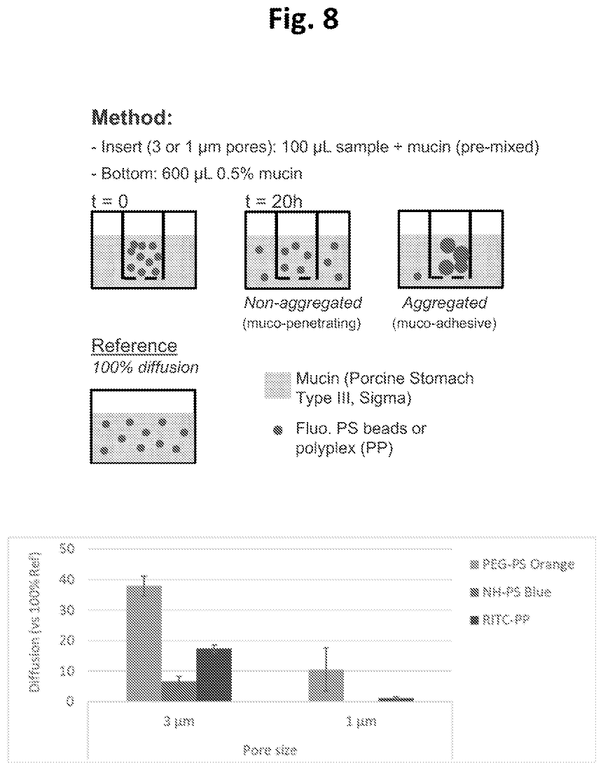

As shown in FIG. 8, in 0.4% Mucin, PEGylated PS beads had the highest level of diffusion after 20 hour incubation through either 3 um or 1 um PET membrane, compared to amine modified PS beads and RITC-labelled polyplex. This was due to less aggregation of PEGylated PS beads in mucin.

### 16. Diffusion of PEGylated Polyplex Formulations Through Type III Mucin Using Transwells Repeat and Effect of Pluronics F127.

16.1 Materials

| Material | Additional Information |
|---|---|
| Amine-modified PS beads, orange, | Orange (Ex: 481, Em: 644), |
| 100 nm, 2.5% w/w | |
| PEGylated PS beads, orange, | Orange (Ex: 481, Em :644) |
| 1.25% w/w | |
| Amine-modified PS beads, blue, | Blue (Ex: 360, Em: 420) |
| 50 nm, 2.5% w/w | |
| PEGylated PS beads, blue, 2.5% w/w | Blue (Ex: 360, Em: 420) |
| RITC-DD-X + DNA Polyplex | |
| (NP7, 0.25 mg/mL) | |
| PEGylated RITC-DD-X + DNA | |
| Polyplex, N:P:A 7/1/1.75 | |
| PEGylated RITC-DD-X + DNA | |
| Polyplex, N:P:A 7/1/2.5 | |
| PEGylated RITC-DD-X + DNA | |
| Polyplex, N:P:A 7/1/3.2 | |
| PEGylated RITC-DD-X + DNA | |
| Polyplex, N:P:A 7/1/1.75 | |
| PEGylated RITC-DD-X + DNA | |
| Polyplex, N:P:A 7/1/2.5 | |
| PEGylated RITC-DD-X + DNA | |
| Polyplex, N:P:A 7/1/3.2 | |
| Mucin from porcine stomach, type 3 | |
| 24 well suspension culture plate | |
| (hydrophobic) | |
| HTS multiwell insert system with | |
| 3 um PET membrane | |
| Trehalose Dihydrate | |
| IbiTreat U-slide VI | |
| Pluronics F127 | |

16.2 Equipment

| Description | Manufacturer |
|---|---|
| Plate reader, EnVision 2105 | Perkin Elmer |
| Analytical Balance | Mettler Toledo |
| Incubator, model 3110 | ThermoFisher |
| Microscope, EVOS FL Auto | Thermofisher |

16.3 Procedure

### 4% Pluronics F127 Solution Preparation

Pluronics F127 was dissolved in water to attain 4% w/w and 0.22 um filtered.

Preparation of Type III Mucin suspension

Type III mucin suspension was prepared as described herein.

### Aggregation in Mucin Test

Test samples (PEGylated RITC-DD-X+DNA Polyplex, RITC-labelled polyplex, amine modified PS beads, or PEGylated PS beads) were combined with Type III mucin suspensions in microtubes at various concentrations to attain final target concentrations of the test sample and mucin. The sample-mucin samples were protected from light and mixed at 30 rpm at room temperature. At 30 and 60 minutes, the mixtures were aliquoted to an Ibidi u-slide and observed under an EVOS microscope for visible aggregation.

### Evaluation of Diffusion Through Mucin-Loaded Transwells

### Sample Preparation.

Test samples (PEGylated RITC-DD-X+DNA Polyplex, RITC-labelled polyplex) were combined with Type III mucin suspensions with or without Pluronics F127 solution in microtubes to attain final target concentrations of the test sample and final mucin concentration of 0.5%. Controls (amine modified PS beads, PEGylated PS beads) were combined mucin only, to final mucin concentration of 0.5%. Samples were protected from light and mixed at 40 rpm for 1 hour prior to loading in transwell insert.

### Transwell Preparation and Sample Diffusion

Transwell bottoms were filled 0.6 mL of 0.5% Type III mucin. HTS transwell inserts with 3 um PET membranes were loaded with 0.1 mL of pre-mixed sample+mucin. Reference (to mimic 100% diffusion) was pre-mixed sample+mucin without a transwell insert (0.7 mL). After 20 hours incubation at 37° C., the diffusion of test samples into the bottom wells was evaluated by fluorescence plate reader (Envision).

16.4 Results

### PEGylation of PS Beads

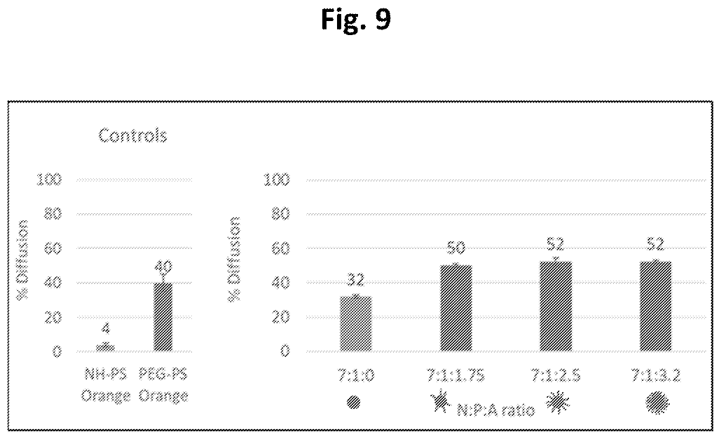

PEGylation of amine modified PS beads improved mucin diffusion nearly 10-fold (FIG. 9). Fluorescence microscopy showed PEGylated PS beads did not aggregate in mucin, whereas non-PEGylated PS beads were severely aggregated under the same conditions.

### PEGylated Polyplex Diffusion in Mucin

PEGylation of polyplex (N:P 7) increases mucin diffusion from 32% to 50-52%, which is about a 60% improvement of diffusion over non-PEGylated polyplex (FIG. 9). The tested N:P:A ratio performed similarly for mucin diffusion. Fluorescence microscopy showed no significant morphological difference between PEGylated and non-PEGylated polyplexes.

### Effect of Pluronics F127 on Mucin Diffusion

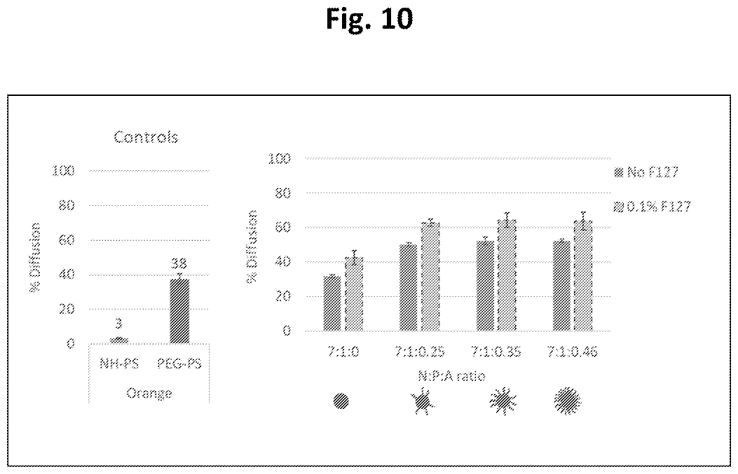

The presence of Pluronics F127 improved mucin diffusion (10-15%) of non-PEGylated polyplex and PEGylated polyplex (FIG. 10).

17. Freeze-Drying of PEGylated Polyplexes Prepared at c125, at Various NPA Ratios, in 2.5 or 5% Trehalose

### Reference: CMC-INT06-EXP-103

17.1 Lyophilization materials

| Material | Cat no. |
|---|---|
| 2 mL vials | 223683 |
| Chlorobutyl lyophilization | 71000-060 |
| stoppers, 13 mm | |
| 13 mm aluminum seals | Z114138-100EA |
| 10 mL vials | 06-406-38 |
| Chlorobutyl lyophilization | 89079-400 |
| stoppers, 20 mm | |
| Aluminum Closed-top crimp | Z114146-100EA |
| seals, 20 mm | |
| 150 mL bottle top 0.22 um | 431161 |
| PES filter | |

17.2 Lyophilization equipment

| Item | Supplier |
|---|---|
| Lyostar II lyophiliser | FTS |
| Stainless steel trays | SP scientific |
| Oven, model 0F-02 | Jeio Tech |
| Autoclave | N/A |

17.3 Procedure

DD-X—DNA (pVax-PD-L1-Fc) polyplexes were produced at N:P ratio of 7:1 and DNA concentration of 0.25 mg/mL DNA with 5% trehalose as previously described. Aliquots of the polyplex were then PEGylated (mPEG5K-b-PLE10) as previously described. The resulting formulations in addition to the N:P 7 polyplex, were generated and filled into 2 mL glass vials (1 mL and 0.1 mL fill volumes were tested).

| Sample ID | NPA ratio | Comments |
|---|---|---|
| CMC-INT06-098 A | 7:1:0 | DD-X-DNA (pVax-PD-L1-Fc) polyplex, |
| | | 5% trehalose, 0.25 mg/mL |
| CMC-INT06-098 B | 7:1:0.88 | PEGylated with PEG-PGA |
| CMC-INT06-098 C | 7:1:1.25 | (mPEG5K-b-PLE10), 5% trehalose |
| CMC-INT06-098 D | 7:1:1.75 | |
| CMC-INT06-098 E | 7:1:3.5 | |
| CMC-INT06-098 F | 7:1:3.5 | PEGylated with PEG-PGA |
| | | (mPEG5K-b-PLE10), 2.5% trehalose |

Samples were lyophilized using the following controlled freeze drying cycle:

Freezing: From +20 to −40° C. (at 1° C./min), then 120 min at −40° C.
Primary Drying: 3800 min at −32° C. & 63 mTorr
Secondary Drying: P=63 mTorr, shelf T from −32 to 30° C. (0.2° C./min), then 360 min at 30° C.

At the end of secondary drying, the vials were purged with nitrogen gas, stoppered, and allowed to equilibrate to room temperature. Short term stability was performed at room temperature (ambient humidity), at room temperature in a desiccator, and at 4° C. At selected timepoints, the 1 mL samples were rehydrated with water and incubated for 15 minutes before analysis. Samples were analyzed for particle size, PDI, zeta potential, and free DNA as described herein.

17.4 Results

Freeze dry cakes had no signs of collapse or cracking. The 0.1 mL samples were shaped like rings due to insufficient volume to form a cake. No free DNA was observed in re-hydrated samples.

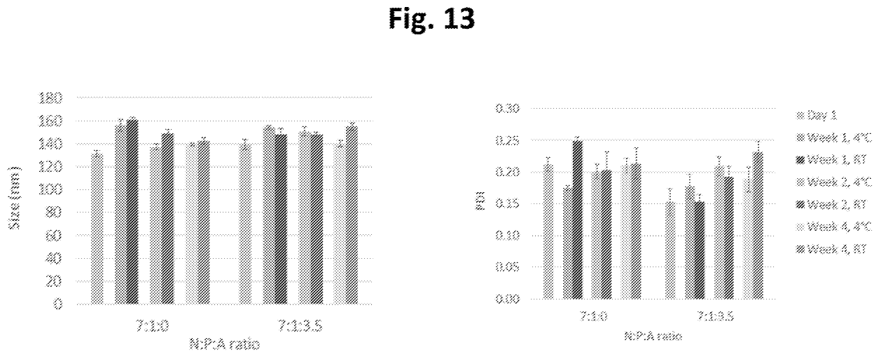

FIG. 13 shows the stability of freeze-dried PEGylated polyplex at room temperature and 4° C. up to 4 weeks. Samples were re-hydrated to pre-freeze dry volume.

### 18. Screening of Potential Freeze-Drying Excipients for In-Line Mixed PEG-PP by Freeze-Thaw

18.1 Excipient screening materials

| Material | Cat. No. |
|---|---|
| Mannitol | M4125-100G |
| Kollidon 12 PF (PVP 2 kDa) | 50348141 |
| PEG 4 kDa | 95904-250G-F |

18.2 Procedure

### Preparation of Mannitol Solution

Generally, mannitol was dissolved in water at a concentration of 0.125 g/mL. The resulting solution was filtered using a 0.2 μm filter. If necessary, the mannitol solution was diluted to required concentration necessary for subsequent formulations.

### Preparation of Kollidon 12 (PVP 2) Solution

Generally, PVP 2 was dissolved in water at a concentration of 0.05 g/mL. The resulting solution was filtered using a 0.2 μm filter. If necessary, the PVP 2 solution was diluted to required concentration necessary for subsequent formulations.

### Preparation of PEG 4 kDa Solution

Generally, PEG 4 kDa was dissolved in water at a concentration of 0.05 g/mL. The resulting solution was filtered using a 0.2 μm filter. If necessary, the PEG 4 kDa solution was diluted to required concentration necessary for subsequent formulations.

### Preparation of Formulations

DD-X—DNA (pVax-PD-L1-Fc) polyplex were produced at N:P ratio of 7:1 and DNA concentration of 0.25 mg/mL DNA with no other excipient, as previously described. Aliquots of the polyplex were then PEGylated (mPEGSK-b-PLE10) as described herein.

| | | | Excipient | |
|---|---|---|---|---|
| | | | Concentration | |
| Sample ID | NPA ratio | Excipient | (% w/w) | Comments |
| CMC-INT06-112A | 7:1:0 | None | 0 | DD-X-DNA (pVax-PD- |
| | | | | L1-Fc) polyplex, no |
| | | | | excipient, 0.25 mg/mL |
| CMC-INT06-112B | 7:1:17.5 | None | 0 | CMC-INT06-112A |
| | | | | PEGylated with PEG-PGA |
| | | | | (mPEG5K-b-PLE10), 0.125 |
| | | | | mg/mL, no excipient |

Aliquots of the non-PEGylated polyplex (CMC-INT06-112A) or PEGylated polyplex (CMC-INT06-112B) were mixed with either water or different excipients at various concentrations to attain the final target excipient concentrations described below and final DNA concentration of 0.1 mg/mL.

| | | | Excipient | |
|---|---|---|---|---|
| | | | Concentration | |
| Sample ID | NPA ratio | Excipient | (% w/w) | Comments |
| CMC-INT06-112 A1 | 7:1:0 | None | 0 | CMC-INT06-112A diluted |
| | | | | to 0.1 mg/mL with water |
| CMC-INT06-112 B1 | 7:1:17.5 | None | 0 | CMC-INT06-112B diluted |
| | | | | to 0.1 mg/mL with water |
| CMC-INT06-112 B2 | | Trehalose | 2.5 | CMC-INT06-112B diluted |
| CMC-INT06-112 B3 | | | 1 | to 0.1 mg/mL with |
| CMC-INT06-112 B4 | | | 0.5 | trehalose solution |
| CMC-INT06-112 B5 | | Mannitol | 2.5 | CMC-INT06-112B diluted |
| CMC-INT06-112 B6 | | | 1 | to 0.1 mg/mL with mannitol |
| CMC-INT06-112 B7 | | | 0.5 | solution |
| CMC-INT06-112 B8 | | PEG 4 kDa | 1 | CMC-INT06-112B diluted |
| | | | | to 0.1 mg/mL with PEG |
| CMC-INT06-112 B9 | | | 0.5 | 4 Da solution |
| CMC-INT06-112 B10 | | PVP 2 | 1 | CMC-INT06-112B diluted |
| | | | | to 0.1 mg/mL with PVP 2 |
| CMC-INT06-112 B11 | | | 0.5 | solution |

The resulting formulations were aliquoted and either: filled into cryovials and frozen at −80 C; or lyophilized as described herein. Thawed samples were analyzed for particle size, PDI, zeta potential, pH, % supercoil DNA and free DNA, as described herein. Lyophilized samples were assigned separate sample ID as provided below.

| | | | Excipient | |
|---|---|---|---|---|
| | | | Concentration | |
| Sample ID | NPA ratio | Excipient | (% w/w) | Comments |
| CMC-INT06-116 A | 7:1:0 | None | 0 | CMC-INT06-112A1 |
| | | | | lyophilized |
| CMC-INT06-116 B1 | 7:1:17.5 | None | 0 | CMC-INT06-112B1 |
| | | | | lyophilized |
| CMC-INT06-116 B2 | | Trehalose | 2.5 | CMC-INT06-112B2 to |
| CMC-INT06-116 B3 | | | 1 | CMC-INT06-112B4 |
| CMC-INT06-116 B4 | | | 0.5 | lyophilized |
| CMC-INT06-116 B5 | | Mannitol | 2.5 | CMC-INT06-112B5 to |
| CMC-INT06-116 B6 | | | 1 | CMC-INT06-112B7 |
| CMC-INT06-116 B7 | | | 0.5 | lyophilized |
| CMC-INT06-116 B8 | | PEG 4 kDa | 1 | CMC-INT06-112B8 to |
| CMC-INT06-116 B9 | | | 0.5 | CMC-INT06-112B9 |
| | | | | lyophilized |
| CMC-INT06-116 B10 | | PVP2 | 1 | CMC-INT06-112B10 to |
| CMC-INT06-116 B11 | | | 0.5 | CMC-INT06-112B11 |
| | | | | lyophilized |

Lyophilized samples were rehydrated with water to the initial concentration before drying, and analyzed for particle size, PDI, zeta potential, pH, % supercoil DNA and free DNA, as described herein.

18.3 Results

Non-PEGylated polyplex in water only (CMC-INT06-112 A1) was severely aggregated after freeze-thaw. Consequently, no further testing was performed on this sample.

The PEGylated polyplex formulations in water or any of the excipient or concentration tested were translucent and other physicochemical properties were unchanged from before freeze-thaw.

| | | Excipient | | | | | | | | |
|---|---|---|---|---|---|---|---|---|---|---|
| | NPA | | % | | | Size | | ZP | % | Free |
| Sample ID | Ratio | Type | w/w | Appearance | pH | (nm) | PDI | (mV) | SC | DNA |
| CMC- | 7:1:0 | None | 0 | Aggregation | 5.94 | N/D | N/D | N/D | N/D | N/D |
| INT06-112 | | | | | | | | | | |
| A1 | | | | | | | | | | |
| CMC- | 7:1:17.5 | None | 0 | Translucent | 7.14 | 143 | 0.142 | −3.1 | 74 | None |
| INT06-112 | | | | | | | | | | Visible |
| B1 | | | | | | | | | | |
| CMC- | | Trehalose | 2.5 | | 7.14 | 144 | 0.141 | −3.9 | 73 | |
| INT06-112 | | | | | | | | | | |
| B2 | | | | | | | | | | |
| CMC- | | | 1.0 | | 6.99 | 144 | 0.145 | −3.5 | 75 | |
| INT06-112 | | | | | | | | | | |
| B3 | | | | | | | | | | |
| CMC- | | | 0.5 | | 7.09 | 141 | 0.157 | −3.7 | 75 | |
| INT06-112 | | | | | | | | | | |
| B4 | | | | | | | | | | |
| CMC- | | Mannitol | 2.5 | | 7.17 | 143 | 0.151 | −3.0 | 78 | |
| INT06-112 | | | | | | | | | | |
| B5 | | | | | | | | | | |
| CMC- | | | 1.0 | | 6.97 | 143 | 0.142 | −3.5 | 78 | |
| INT06-112 | | | | | | | | | | |
| B6 | | | | | | | | | | |
| CMC- | | | 0.5 | | 6.98 | 143 | 0.155 | −5.1 | 78 | |
| INT06-112 | | | | | | | | | | |
| B7 | | | | | | | | | | |
| CMC- | | PEG | 1.0 | | 7.08 | 144 | 0.145 | −3.4 | 76 | |
| INT06-112 | | 4 kD | | | | | | | | |
| B8 | | | | | | | | | | |
| CMC- | | | 0.5 | | 6.90 | 142 | 0.155 | −3.1 | 77 | |
| INT06-112 | | | | | | | | | | |
| B9 | | | | | | | | | | |
| CMC- | | PVP | 1.0 | | 6.97 | 140 | 0.158 | −3.4 | 76 | |
| INT06-112 | | 2 kD | | | | | | | | |
| B10 | | | | | | | | | | |
| CMC- | | | 0.5 | | 7.07 | 140 | 0.165 | −3.8 | 78 | |
| INT06-112 | | | | | | | | | | |
| B11 | | | | | | | | | | |

### Appearance of Lyophilized Cakes.

The lyophilized cake of the non-PEGylated polyplex in water only (CMC-INT06-116 A1) had inconsistent appearance and was collapsed. The lyophilized cake of the PEGylated polyplex formulations in water or any of the excipient or concentration tested (CMC-INT06-116 B1 to CMC-INT06-116 B11) were uniform, and had no cracking with minor shrinkage.

Physicochemical Properties of Lyophilized Samples after Rehydration

Non-PEGylated polyplex in water only (CMC-INT06-116 A1) was severely aggregated after rehydration. Consequently, no further testing was performed on this sample. The PEGylated polyplex formulations in water or any of the excipient or concentration tested were translucent and other physicochemical properties were unchanged from before lyophilization.

| | | Excipient | | | | | | | |
|---|---|---|---|---|---|---|---|---|---|
| Sample | NPA | | % | | Size | | ZP | % | Free |
| ID | Ratio | Type | w/w | Appearance | (nm) | PDI | (mV) | SC | DNA |
| CMC- | 7:1:0 | None | 0 | Precipitation | N/D | N/D | N/D | N/D | N/D |
| INT06- | | | | | | | | | |
| 116 A1 | | | | | | | | | |
| CMC- | 7:1:17.5 | None | 0 | Translucent | 141.7 | 0.159 | −4.0 | 92 | None |
| INT06- | | | | | | | | | visible |
| 116 B1 | | | | | | | | | |
| CMC- | | Trehalose | 2.5 | | 145.3 | 0.149 | −3.2 | 87 | |
| INT06- | | | | | | | | | |
| 116 B2 | | | | | | | | | |
| CMC- | | | 1.0 | | 140.9 | 0.168 | −3.8 | 89 | |
| INT06- | | | | | | | | | |
| 116 B3 | | | | | | | | | |
| CMC- | | | 0.5 | | 144.7 | 0.149 | −2.9 | 91 | |
| INT06- | | | | | | | | | |
| 116 B4 | | | | | | | | | |
| CMC- | | Mannitol | 2.5 | | 144.8 | 0.148 | −3.0 | 93 | |
| INT06- | | | | | | | | | |
| 116 B5 | | | | | | | | | |
| CMC- | | | 1.0 | | 142.6 | 0.168 | −2.7 | 92 | |
| INT06- | | | | | | | | | |
| 116 B6 | | | | | | | | | |
| CMC- | | | 0.5 | | 146.1 | 0.185 | −2.5 | 92 | |
| INT06- | | | | | | | | | |
| 116 B7 | | | | | | | | | |
| CMC- | | PEG | 1.0 | | 144.2 | 0.151 | −2.7 | 91 | |
| INT06- | | 4 kD | | | | | | | |
| 116 B8 | | | | | | | | | |
| CMC- | | | 0.5 | | 145.0 | 0.161 | −2.3 | 89 | |
| INT06- | | | | | | | | | |
| 116 B9 | | | | | | | | | |
| CMC- | | PVP | 1.0 | | 144.4 | 0.153 | −2.6 | 80 | |
| INT06- | | 2 kD | | | | | | | |
| 116 | | | | | | | | | |
| B10 | | | | | | | | | |
| CMC- | | | 0.5 | | 144.1 | 0.164 | −3.0 | 84 | |
| INT06- | | | | | | | | | |
| 116 | | | | | | | | | |
| B11 | | | | | | | | | |

18.4 Conclusion

PEGylated polyplex at N:P:A ratio=7:1:17.5 using PGA(1.3 k)-PEG(5 k) prevented polyplex aggregation following freeze-thaw or lyophilization in absence of excipients. Trehalose, mannitol, PEG 4 kDa or PVP 2 kDa are not required to prevent PEGylated polyplex aggregation upon freeze-thaw or lyophilization, and they do not cause aggregation at the tested concentrations

### 19. Lyophilization of Concentrated and Diluted Pegylated Polyplex in 5% Trehalose

### Reference: CMC-INT06-EXP-110 & CMC-INT06-128

19.1 Procedure

DD-X—DNA (pVax-PD-L1-Fc) polyplex were produced at N:P ratio of 7:1 and DNA concentration of 0.25 mg/mL DNA with or without 5% trehalose, as previously described. Aliquots of the polyplex were then PEGylated (mPEG5K-b-PLE10) and then concentrated using TFF process as previously described. The resulting formulations in addition to the N:P 7 polyplex, were generated and filled into 2 or 10 mL glass vials (0.3 mL, 1 mL and 2 mL fill volumes were tested).

| Sample ID | NPA ratio | Comments |
|---|---|---|
| CMC-INT06-099 A | 7:1:0 | DD-X-DNA (pVax-PD-L1-Fc) polyplex, |
| | | 5% trehalose, 0.25 mg/mL |
| CMC-INT06-099 B | 7:1:17.5 | PEGylated with PEG-PGA |
| | | (mPEG5K-b-PLE10), |
| | | 5% trehalose, 0.125 mg/mL |
| CMC-INT06-099 C | 7:1:17.5 | CMC-INT06-099 B concentrated |
| | | to 1 mg/mL by TFF |
| CMC-INT06-128 A | 7:1:17.5 | PEGylated with PEG-PGA |
| | | (mPEG5K-b-PLE10), |
| | | no excipient, 1 mg/mL |
| CMC-INT06-128 B | 7:1:17.5 | PEGylated with PEG-PGA |
| | | (mPEG5K-b-PLE10), |
| | | no excipient, 2.5 mg/mL |

Samples were lyophilized using the following controlled freeze drying cycle:

Freezing: From +20 to −40° C. (at 1° C./min), then 120 min at −40° C.
Primary Drying: 3800 min at −32° C. & 63 mTorr
Secondary Drying: P=63 mTorr, shelf T from −30 to 30° C. (0.2° C./min), then 6 hours at 30° C.

At the end of secondary drying, the vials were purged with nitrogen gas, stoppered, and allowed to equilibrate to room temperature. The samples were rehydrated with water to attain different fold-concentrations: 1-fold or 10-fold). Samples were analyzed for particle size, PDI, as described herein.

19.2 Results

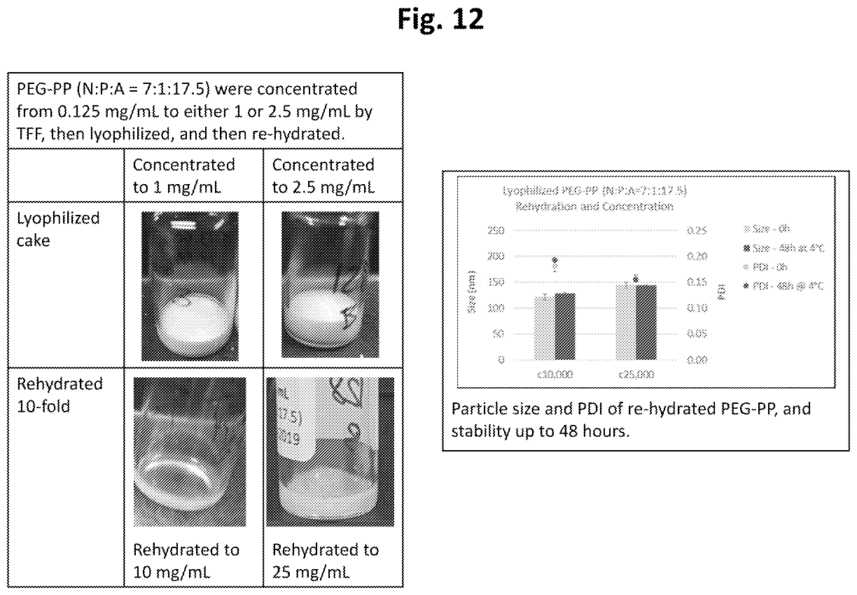

Freeze-dried samples had cakes with firm appearance, light shrinkage and cracks in the 1 mL fill volumes. Freeze-dried samples re-hydrated to 1 mg/mL and 10 mg/mL were translucent in appearance, with no visible aggregates. Freeze-dried sample re-hydrated to 25 mg/mL formed a viscous gel, due to insufficient volume of water to form a suspension (FIG. 12). Freeze-dried sample re-hydrated to 50 mg/mL formed a paste, due to insufficient volume of water to form a suspension.

Freeze-dried samples re-hydrated to 10 mg/mL and 25 mg/mL were tested for nanoparticle size, PDI, and zeta potential, with no apparent aggregation in either condition for at least 48 hours at 4° C. (FIG. 12).

### 20. Freeze-Drying of PEG-PP (Various NPA Ratios) Prepared in 4.5% Sucrose or in Water

### Reference: CMC-INT06-129

20.1 Procedure

DD-X—DNA (pVax-PD-L1-Fc) polyplex were produced at N:P ratio of 7:1 and DNA concentration of 0.125 mg/mL DNA with no other excipient, as previously described. Aliquots of the polyplex were then PEGylated (mPEG5K-b-PLE10) as described herein. Additional test samples CMC-INT06-124 A and CMC-INT06-124 B have already been described herein.

| Sample ID | Source Material | NPA ratio | Comments |
|---|---|---|---|
| CMC-INT06-129A | CMC-INT06-127 A | 7:1:17.5 | DD-X-DNA (pVax-PD-L1-Fc) |
| CMC-INT06-129B | CMC-INT06-127 B | 7:1:12 | polyplex, no excipient, 0.125 mg/mL, |
| CMC-INT06-129C | CMC-INT06-127 C | 7:1:7 | PEGylated with PEG-PGA |
| | | | (mPEG5K-b-PLE10). |
| CMC-INT06-129D | CMC-INT06-124 A | 7:1:17.5 | PEGylated with PEG-PGA |
| CMC-INT06-129E | CMC-INT06-124 B | 7:1:7 | (mPEG5K-b-PLE10), 4.5% sucrose, |
| | | | 0.5 mg/mL DNA |

The resulting formulations were filled into 2 mL glass vials (0.3 mL and 1 mL fill volumes were tested) and lyophilized using the following controlled freeze drying cycle:

Freezing: From 20 to −40° C. (at 1° C./min), then 120 min at −40° C.
Primary Drying: 3660 min at −35° C. & 63 mTorr
Secondary Drying: P=63 mTorr, shelf T from −35 to 30° C. (0.2° C./min), then 360 min at 30° C.

At the end of secondary drying, the vials were purged with nitrogen gas, stoppered, and allowed to equilibrate to room temperature, and stored at 4° C. until use.

Short term stability was performed at 4° C. for the no excipient samples (CMC-INT06-129 A, CMC-INT06-129 B, CMC-INT06-129 C). The 4.5% sucrose samples (CMC-INT06-129 D and CMC-INT06-129 E) were tested at time 0. At selected timepoints, the samples were rehydrated with water to attain the concentration before lyophilization. Re-hydrated samples were analyzed for particle size, PDI, and zeta potential, as described herein.

20.2 Results

All formulations were successfully lyophilized. Formulations without lyoprotectant showed retraction/radial contraction; those with lower NPA ratio (i.e. NPA=7:1:7) and lower fill volume (0.3 mL) were more susceptible to static (cake moving up, sideways, breaking, etc.). Formulations containing 4.5% sucrose showed limited retraction only and had a glossy surface.

### Description of Lyophilized Samples

| | Volume | | | |
|---|---|---|---|---|
| | Format | N:P:A | | |
| Sample Name | (uL) | ratio | Excipient | Appearance |
| CMC-INT06-129A | 300 | 7:1:17.5 | None | White, radial contraction/retraction, |
| | | | | shiny textured surface |
| CMC-INT06-129A | 1000 | 7:1:17.5 | None | White, radial contraction/retraction, |
| | | | | shiny textured surface |
| CMC-INT06-129B | 300 | 7:1:12 | None | White, radial contraction/retraction, |
| | | | | shiny textured surface |
| CMC-INT06-129B | 1000 | 7:1:12 | None | White, radial contraction/retraction, |
| | | | | shiny textured surface |
| CMC-INT06-129C | 300 | 7:1:7 | None | White, radial contraction/retraction, |
| | | | | shiny textured surface, lots of |
| | | | | static/easily moves up or sideways, |
| | | | | folds, or breaks. |
| CMC-INT06-129C | 1000 | 7:1:7 | None | White, radial contraction/retraction, |
| | | | | shiny textured surface, lots of |
| | | | | static/moves up |
| CMC-INT06-129D | 300 | 7:1:17.5 | 4.5% Suc | White, limited radial |
| | | | | contraction/retraction, glossy surface |
| CMC-INT06-129E | 300 | 7:1:7 | 4.5% Suc | White, limited radial |
| | | | | contraction/retraction, glossy surface |

The appearance, particle size, PDI, and zeta potential of the rehydrated samples at time 0.

| | | Particle | | Zeta | |
|---|---|---|---|---|---|
| | | Size | | Potential | |
| Sample ID | Appearance | (nm) | PDI | (mV) | Comments |
| CMC-INT06- | Clear/ | 134.8 | 0.139 | −1.1 | Phys chem at |
| 129A | translucent | | | | timepoint 0 |
| CMC-INT06- | Clear/ | 133.2 | 0.138 | −0.3 | Phys chem at |
| 129B | translucent | | | | timepoint 0 |
| CMC-INT06- | Clear/ | 132.9 | 0.128 | −0.2 | Phys chem at |
| 129C | translucent | | | | timepoint 0 |
| CMC-INT06- | Clear/ | 136.8 | 0.117 | −2.1 | Phys chem at |
| 129D | translucent | | | | timepoint 0 |
| CMC-INT06- | Clear/ | 134.9 | 0.121 | 0.8 | Phys chem at |
| 129E | translucent | | | | timepoint 0 |

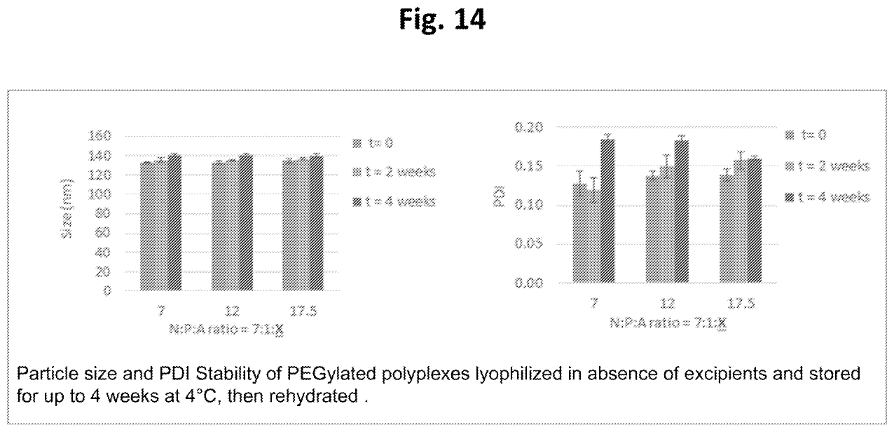

The stability of lyophilized samples at 4° C. up to 4 weeks in the absence of excipients (particle size, PDI, and zeta potential of the rehydrated samples) are shown in FIG. 14. Results showed no change in particle size and zeta potential, and limited or no increase in PDI.

The table below provides a summary of current finding:

| | PEG-PGA | PEG-PGA | PEG-HA | PEG-HA | |
|---|---|---|---|---|---|
| NP Ratio | (1 k-1.3 k) | (5 k-1.3 k) | (2 k-10 k) | (2 k-50 k) | |
| 3 | N:A tried | N:A tried | PEG-HA | Aggregate | Free DNA. |
| | 1000-100. | 1000-30. | forms | (N/:ratios | Polyplexes |
| | Polyplex are | Polyplex are | hydrogel | tried-100, | increase in |
| | not stable | stable after | | 200 and 400) | size after: |
| | after FT | FT. | | | PEGylation |
| 5 | N:A tried | N:A tried | PEG-HA | Aggregate | Slightly |
| | 1000-100. | 1000-30. | forms | (N: A ratios | lower |
| | Polyplex are | Polyplex are | hydrogel | tried-100, | stability |
| | not stable | stable after | | 200 and 400) | compared to |
| | after FT | FT | | | NP7. |
| 7 | N:A tried | N:A tried | PEG-HA | Aggregate | |
| | 1000-100. | 1000-30. | forms | (N:A ratios | |
| | Polyplex are | Polyplex are | hydrogel | tried-100, | |
| | not stable | stable after | | 200 and 400) | |
| | after FT | FT | | | |
| Reason for | Polyplexes | | Polyanion is | Solution is | |
| Elimination | are not stable | | not soluble | too viscous | |

### Example 2

General methods, compositions, and procedures, unless otherwise described, are as described above in Example 1.

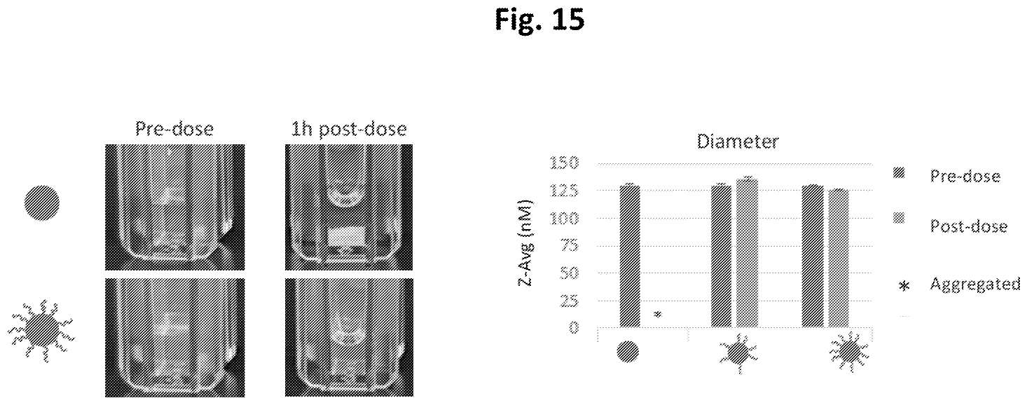

80 μL of reversibly PEGylated dually derivatized chitosan polyplex (DDX), wherein the PEG comprised a polyglutamate tail and the polyplex comprised an NPA ratio of 7:1:7 (medium pegylation) or 7:1:17.5 (high pegylation) were administered to a mouse bladder at 250 μg/mL. As a control, 80 μL of non-PEGylated polyplex at an NP ratio of 7:1 was administered to a mouse bladder. The administered formulations were incubated in the bladder for 1 h, and then the contents of the bladder were collected for analysis. As shown in FIG. 15, incubation in the bladder caused severe aggregation of non-PEGylated polyplex. In contrast, both high and medium PEGylated polyplex showed no detectable aggregation after incubation in the bladder.

### Example 3

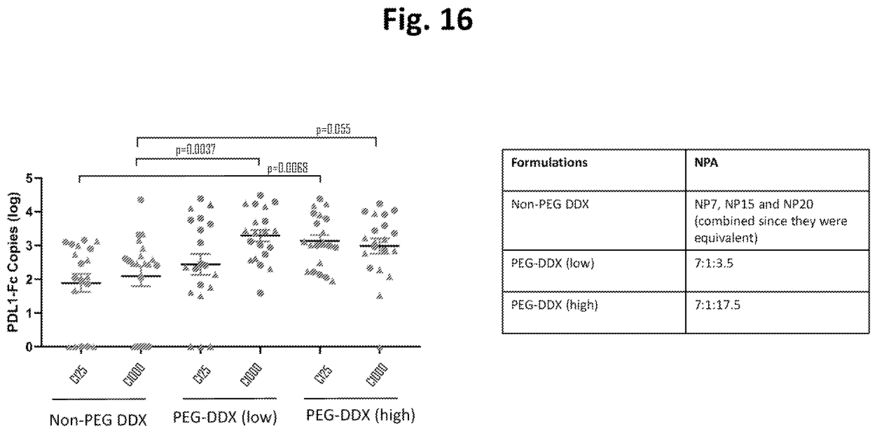

In vivo gene transfer efficiency of reversibly PEGylated and non-PEGylated DDX particles were compared. Non-PEGylated DDX particles at NP7, NP15, and NP20 and PEGylated DDX at NPA 7:1:3.5 (low PEGylation) and NPA 7:1:17.5 (high PEGylation) were tested. The reversibly PEGylated DDX particles were coated with PEG-polyglutamate as described in Examples 1 and 2. Each of the DDX formulations were tested at two different concentrations (125 μg/mL and 1,000 μg/mL). Formulations were injected as 3 150 μL doses by intracolonic instillation (ICI). After 24 hours, colon samples were collected and analyzed for transgene mRNA expression. As shown in FIG. 16, the PEGylated DDX particles achieved remarkably improved gene delivery as detected by the increase in copy number expression (>10×). The % samples with detectable gene expression was also significantly improved in the PEGylated DDX formulations.

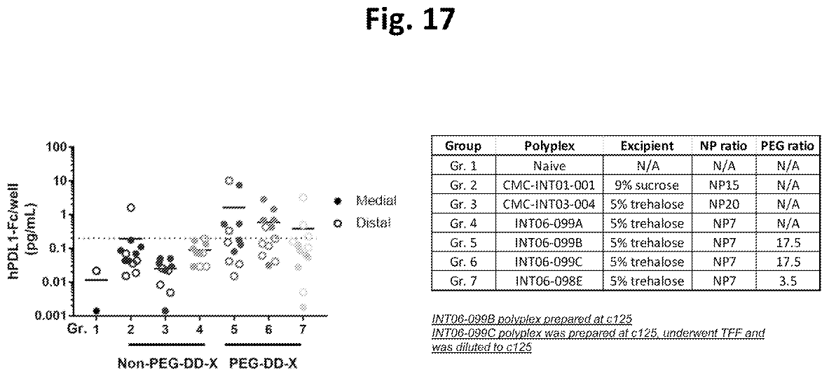

In addition to mRNA expression, expression of the transgene encoded protein was also assessed. Non-PEGylated DDX particles at NP15 (Gr. 2) and NP20 (Gr. 3) and PEGylated DDX at NPA 7:1:3.5 (Gr. 7) and NPA 7:1:17.5 (Gr. 5 and 6) were tested. The reversibly PEGylated DDX particles were coated with PEG-polyglutamate as described in Examples 1 and 2. Each of the DDX formulations were tested at 125 μg/mL. Formulations were injected as 3 150 μL doses by intracolonic instillation (ICI). After 24 hours, cell samples were collected and analyzed for transgene protein expression using a Mesoscale Discovery Immunoassay. As shown in FIG. 17, transgene encoded protein expression was significantly increased in mouse colon treated with PEGylated DDX particles relative to non-PEGylated DDX particles.

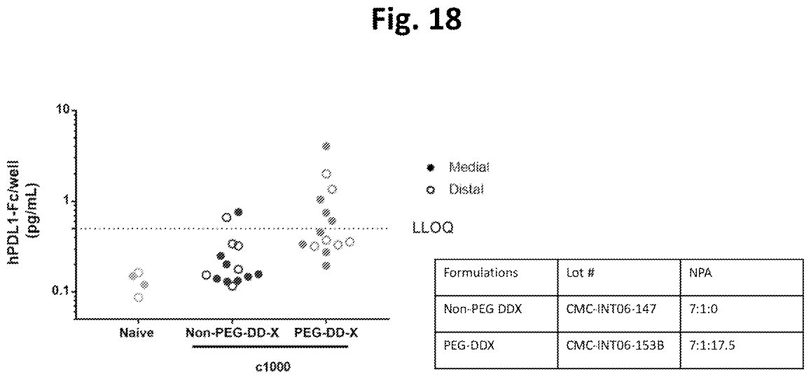

The protein expression experiment was repeated with non-PEGylated DDX particles having an NP ratio of 7:1 and PEGylated DDx particles having an NPA ratio of 7:1:17.5 each formulated at 1000 μg/mL. Formulations were administered using a 3×150 μL ICI procedure; colon sections were harvested at 24 h post-administration and protein lysates were used to quantify human PD-L1-Fc protein using a Mesoscale Discovery immunoassay. As shown in FIG. 18, transgene encoded protein expression was significantly increased in mouse colon treated with PEGylated DDX particles relative to non-PEGylated DDX particles.

### Example 4

PEGylated DDX nanoparticles were produced using the general methods outlined in the foregoing examples and their physico-chemical parameters assayed. Dually derivatized polyplexes having a 25% cation (arginine (R)) final functionalization degree and a 10% polyol (glucose (G)) final functionalization degree were prepared with NP20:1 (non-PEGylated) and NPA10:1:5 (PEGylated) ratios. Lyoprotectant (5% trehalose or 1% mannose) was selected on the basis of the improved lyoprotection provided for the respective non-PEGylated and PEGylated polyplexes. Formulations were frozen or lyophilized and then thawed or rehydrated as applicable. Samples were tested for % supercoil DNA, appearance, particle size, PDI, zeta potential and pH.

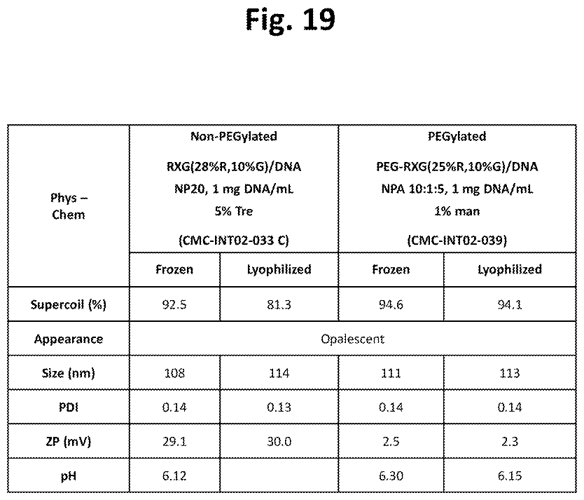

As shown in FIG. 19, at t=0 for non-PEG lyophilized, the % supercoil DNA was significantly lower than the equivalent PEGylated formulation. % supercoil DNA indicates the quality of the nucleic acid delivery material, and preferably should be >80% and more preferably >90%.

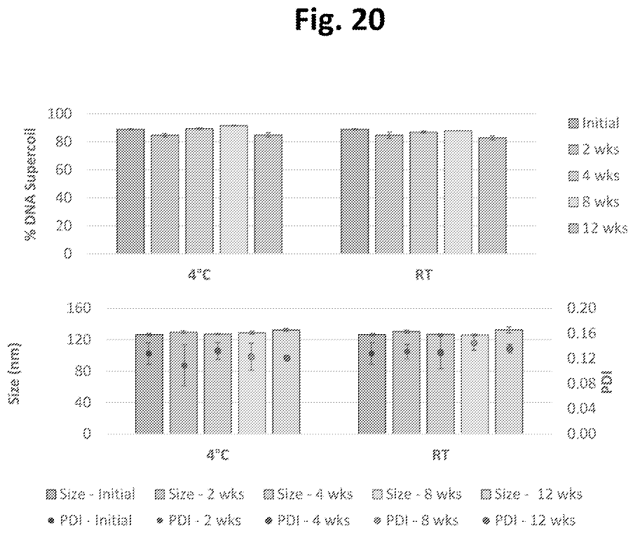

Polyplexes were prepared at an NPA (PEGylated) ratio of 10:1:5. For these polplexes, the cation/polyol final functionalization degree was 28% R and 10% G, and DNA concentration was 125 μg/mL. Lyoprotectant was 1% mannitol. Formulations were lyophilized and stored at 4° C. and room temperature for up to 12 weeks. At t=0 (initial), 2 weeks, 4 weeks, 8 weeks, and 12 weeks, samples were rehydrated to the original DNA concentration and tested for % supercoil DNA, particle size, and PDI. As illustrated in FIG. 20, lyophilized PEGylated DDX formulation was stable for up to 12 weeks when stored at either 4° C. or room temperature (RT).

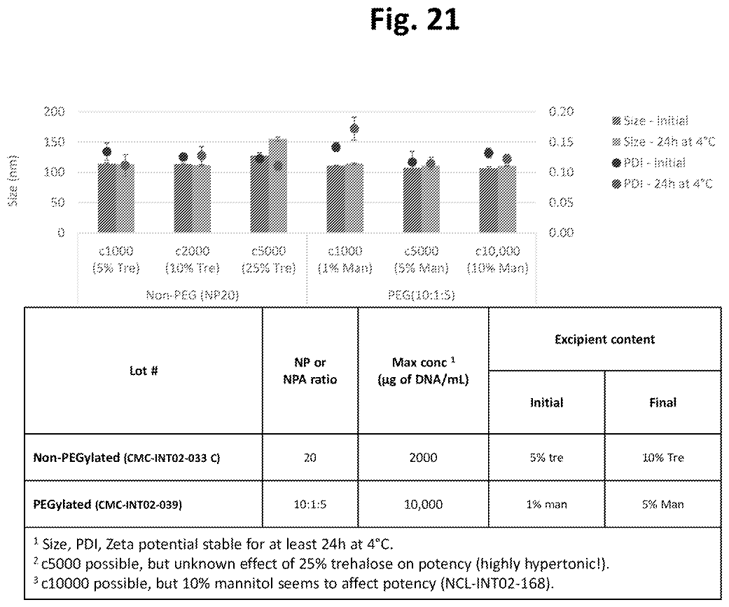

Polyplexes were prepared at an NPA (PEGylated) ratio of 10:1:5 and NP (non-PEGylated) ratio of 20:1. For these polplexes, the cation/polyol final functionalization degree was 28% R and 10% G, and DNA concentration was 1000 μg/mL. Lyoprotectant (5% trehalose or 1% manitol) was selected on the basis of the improved lyoprotection provided for the respective non-PEGylated and PEGylated polyplexes. Formulations were lyophilized and then rehydrated to the target DNA concentrations indicated (c1000=1 mg/mL, c2000=2 mg/mL, c5000=5 mg/mL, c10,000=10 mg/mL). Samples were tested for particle size and PDI. As illustrated in FIG. 21, the lyophilized PEGylated formulation was stably rehydrated at a concentration up to 10 mg/mL, while the non-PEGylated formulation was able to be stably rehydrated to 2 mg/mL. While the, 2 mg/mL rehydration concentration provided a useful gene delivery formulation, the unexpectedly significant increase in the achievably stable DNA concentration (10 mg/mL) of the PEGylated formulation provides significant benefits in terms of required dose volume to achieve a therapeutic or clinically relevant effect.

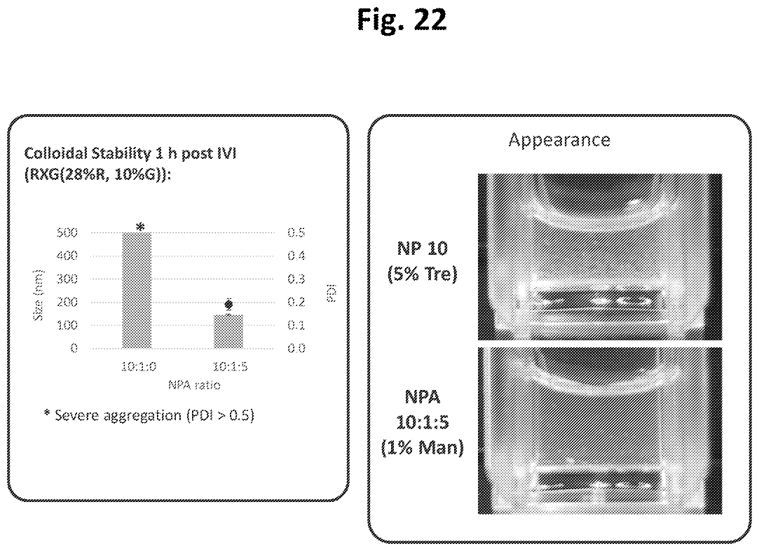

Polyplexes were prepared at an NPA (PEGylated) ratio of 10:1:5 and NP (non-PEGylated) ratio of 10:1. For these polplexes, the cation/polyol final functionalization degree was 28% R and 10% G. Formulations containing 1% mannitol (PEGylated polyplexes) or 5% trehalose (non-PEGylated polyplexes) were administered to the mouse bladder at a DNA concentration of 125 μg/mL, incubated for 1 hour, and the contents of the bladder were collected for analysis. Samples were examined for visual appearance and nanoparticle sizing by dynamic light scattering. As illustrated in FIG. 22, the PEGylated polyplexes exhibited no detectable aggregation, while the non-PEGylated polyplexes exhibited severe aggregation as shown on the left by increase in size and polydispersity index (PDI) and on the right by the appearance of white clots in the image of the collected urine samples.

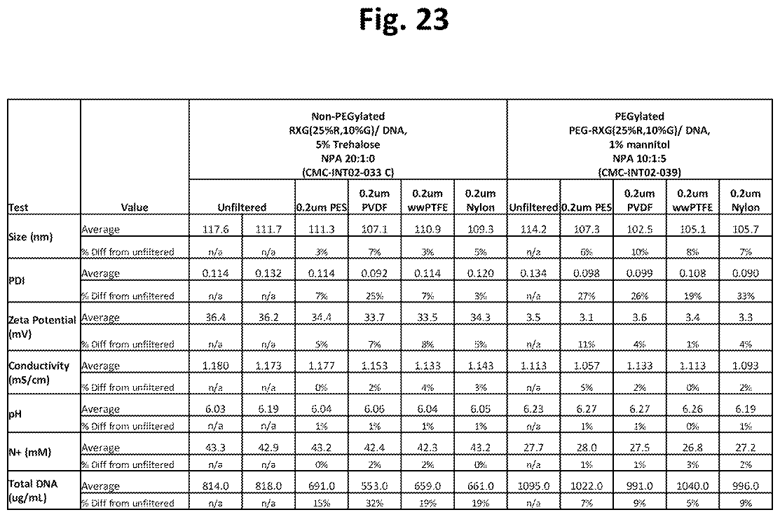

Polyplexes were prepared at an NPA (PEGylated) ratio of 10:1:5 and NP (non-PEGylated) ratio of 20:1. For these polplexes, the cation/polyol final functionalization degree was 28% R and 10% G. Formulations containing 1% mannitol (PEGylated polyplexes) or 5% trehalose (non-PEGylated polyplexes) were assayed for sterile filtration suitability. 0.5 mL of non-PEGylated (NPA 20:1) and PEGylated (NPA 10:1:5) DDX polyplex formulations at 1 mg DNA/mL, were filtered through 0.2 μm pore filter (13 mm diameter, 1 cm² surface area) comprised of different membrane types: PES (Polyethersulfone), PVDF (Polyvinylidene difluoride), PTFE (Polytetrafluoroethylene), and Nylon. Samples were tested for nanoparticle sizing, zeta potential, conductivity, pH, cation (Arginine) and polyol (glucose) content and DNA content. As illustrated in FIG. 23, DNA concentration exhibited a significantly larger decrease (15-32%) after filtration of non-PEGylated polyplex formulations as compared to PEGylated polyplex formulations (<10%). These results suggest that the non-PEGylated polyplexes aggregate under the assay conditions.

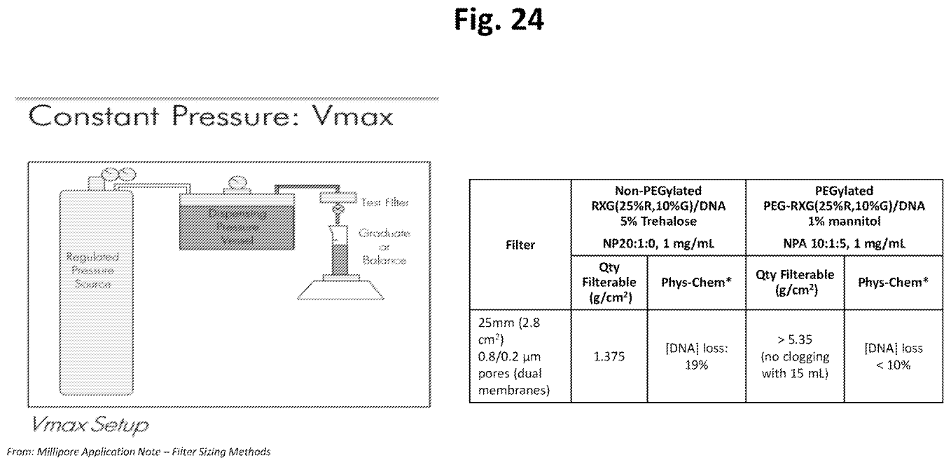

To confirm the results illustrated in FIG. 23, production of PEGylated and non-PEGylated DDX chitosan-DNA polyplexes was scaled up and formulations were tested for filterability in larger volumes. For these polplexes, the cation/polyol final functionalization degree was 28% R and 10% G. Formulations containing 1% mannitol (PEGylated polyplexes) or 5% trehalose (non-PEGylated polyplexes) were assayed for sterile filtration suitability. 5-15 mL of non-PEGylated (NPA 20:1:0) and PEGylated (NPA 10:1:5) DDX DNA polyplex formulations at a DNA concentration of 1 mg DNA/mL, were filtered through a 0.8/0.2 μm pore PES filter stack (25 mm diameter, 2.8 cm² surface area, each filter). Filtration was via a constant pressure Vmax filtration set-up at 30 psig (schematic shown in FIG. 24). Filterability was determined as filtrate mass collected per surface area. Pre- and post-filtration samples were tested for nanoparticle sizing, zeta potential, conductivity, pH, cation/polyol content, and DNA content.

As illustrated in FIG. 24, DNA concentration exhibited a significantly larger decrease (19%) after filtration of non-PEGylated polyplex formulations as compared to PEGylated polyplex formulations (<10%). Moreover, the non-PEGylated polyplex formulation clogged the filtration apparatus when supplied at a maximum concentration of 1.375 g of polyplex/cm² membrane surface area. In contrast, PEGylated polyplex did not clog the filter at all polyplex concentrations tested, up to 5.35 g polplex/cm² surface area, suggesting that PEGylated polyplex formulations above 5.35 g/cm² remain filterable.

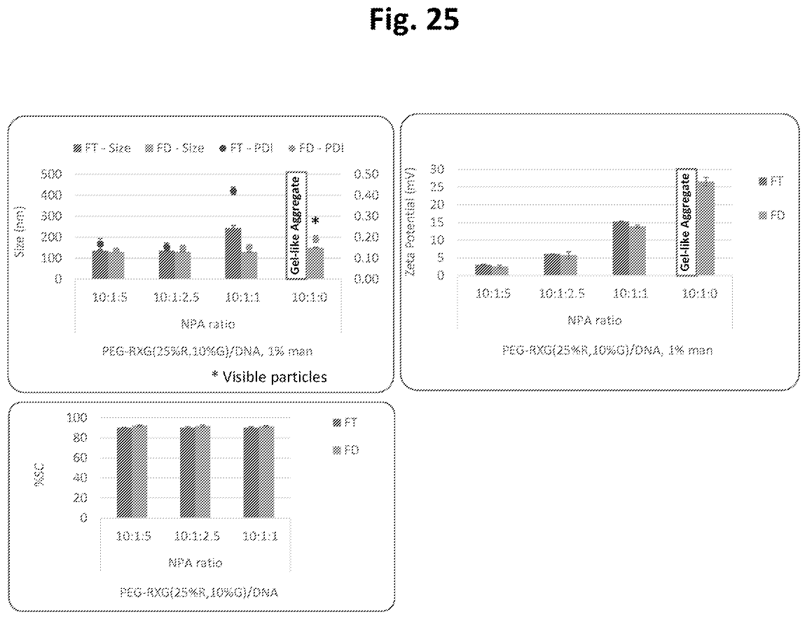

Polyplexes were prepared at NPA (PEGylated) or NP (non-PEGylated) ratios indicated in FIG. 25, and DNA concentrations of 0.125 mg/mL (c125). Cation/polyol number ratios were 25% R and 10% G. Lyoprotectant was 1% mannitol (1% Man). Formulations were freeze-thawed (FT) or lyophilized and rehydrated (FD). Samples were tested for % supercoil DNA nanoparticle size and zeta potential. As illustrated in FIG. 25, the non-PEGylated DDX formulation precipitated after freeze/thaw and lyophilization/rehydration. % supercoil DNA could not be measured on the precipitated sample. In contrast, PEGylated DDX formulations (NPA 10:1:5 and 10:1:2.5) did not aggregate after freeze/thaw, while 10:1:1 formulation indicated some aggregation after freeze/thaw, albeit significantly less than the non-PEGylated material. After lyopholization/rehydration, PEGylated all DDX formulations tested (NPA 10:1:5 to 10:1:1) did not exhibit any detectable aggregation.

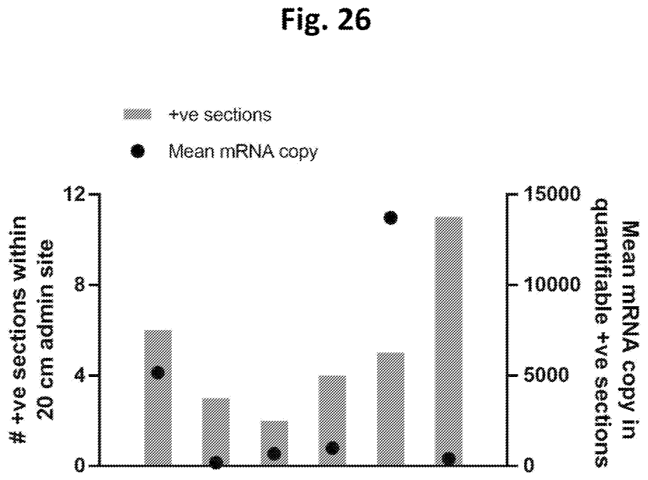

PEGylated DDX polyplexes were prepared and delivered to a dog small intestine by direct instillation. As shown in FIG. 26, mRNA copy number was detected at 24 h post-delivery demonstrating gene delivery in the dog.

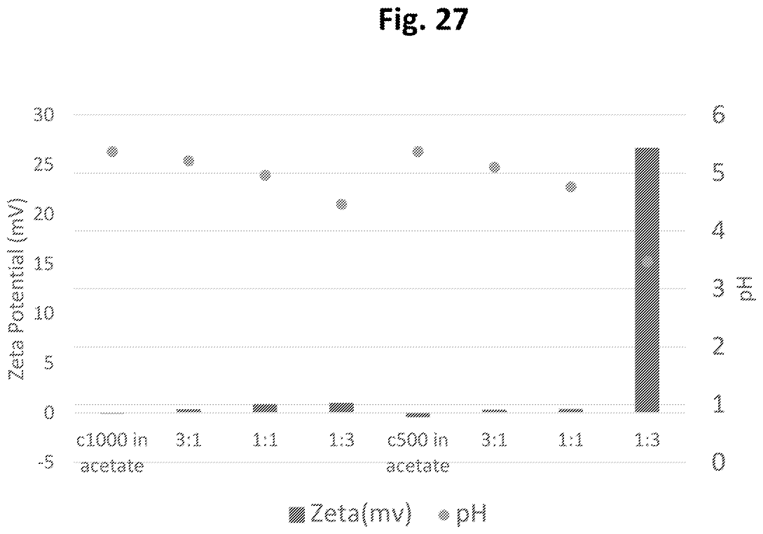

PEGyalted polyplexes were mixed with various ratios of acetate buffer to simulate low pH environments and the pH of the resulting solution was determined. The zeta potential of the particles was also monitored. As the pH was lowered below the pKa of the polymer coat's anionic anchor region (˜4.25), the zeta potential increased dramatically, indicating release of the polymer coat. See, FIG. 27.

### Example 5

Reversibly PEGylated polyplexes containing dually derivatized chitosan (Arg:gluconic acid) were prepared at N:P:A ratios of 7:1:X, wherein X is 3.5, 9, or 14.5, with polyglutamate anchor regions of differing size. PLE5 refers to polyplexes comprising a PEG-polyglutamate (PEG-PLE) polymers, wherein the polyglutamate anchor region is 5 glutamate amino acids in length. PLE10 refers to a 10 glutamate amino acid length, and PLE25 refers to a 25 glutamate amino acid length. Physico-chemical parameters of the resulting PEGylated polyplexes are shown in the Table below.

| | Sample | | | | | | | | |
|---|---|---|---|---|---|---|---|---|---|
| | PLE5 | | | PLE10 | | | PLE25 | | |
| | NPA | | | | | | | | |
| | 7:1:(3.5) | 7:1:9 | 7:1:(14.5) | 7:1:(3.5) | 7:1:9 | 7:1:(14.5) | 7:1:(3.5) | 7:1:9 | 7:1:(14.5) |
| Appearance | C-T | C-T | C-T | C-T | C-T | C-T | C-T | C-T | C-T |
| Size (nm) | 136 | 134 | 131 | 1315 | 131 | 130.9 | 129 | 127 | 130 |
| PDI | 0.15 | 0.16 | 0.18 | 0.15 | 0.17 | 0.17 | 0.17 | 0.15 | 0.12 |
| ZP (mV) | 3.2 | 0.2 | −2.2 | 1.9 | −2.3 | −5.7 | 1.5 | −3.2 | −4.8 |
| pH | 6.10 | 6.42 | 6.66 | 6.29 | 6.9 | 7.23. | 6.49 | 7.22 | 7.49 |
| Osmolality | 181 | 183 | 181 | 190 | 193 | 188 | 188 | 191 | 172 |
| (mmole/kg) | | | | | | | | | |
| % SC | 82 | 81 | 82 | 82 | 81 | 84 | 83 | 84 | 85 |
| Free DNA | None | None | None | None | None | None | None | None | None |
| [DNA] ug/mL | 100 | 104 | 119 | 120 | 100 | 100 | 110 | 96 | 124 |
| [N+] | 2.6 | 2.5 | 2.5 | 2.5 | 2.5 | 2.6 | 2.6 | 2.6 | 2.5 |
| [PEG] mg/mL | 1.93 | 5.2 | 8.13 | 1.37 | 3.05 | 4.68 | N/A | N/A | N/A |
| N:P Ratio | 8 | 8 | 7 | 7 | 8 | 8 | 7 | 9 | 7 |
| (Calculated) | | | | | | | | | |
| A:P Ratio | 4 | 11 | 16 | 5 | 12 | 19 | 8 | 18 | 20 |
| (Calculated) | | | | | | | | | |

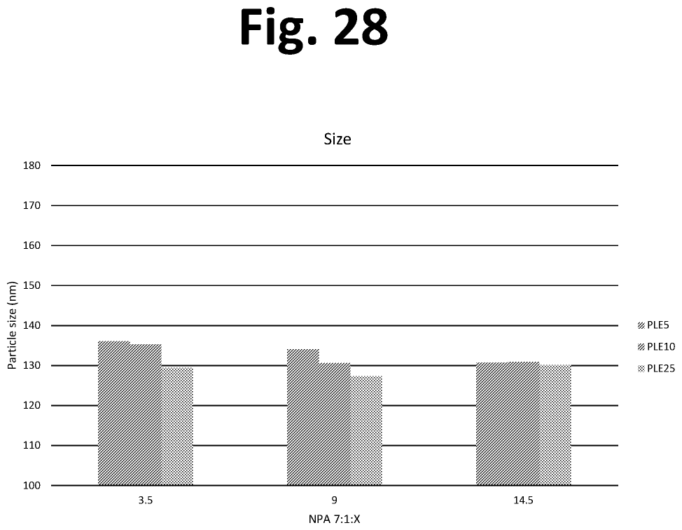

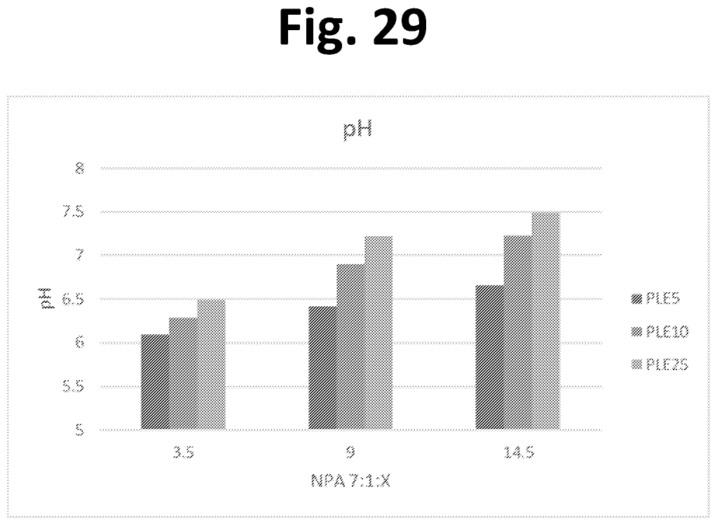

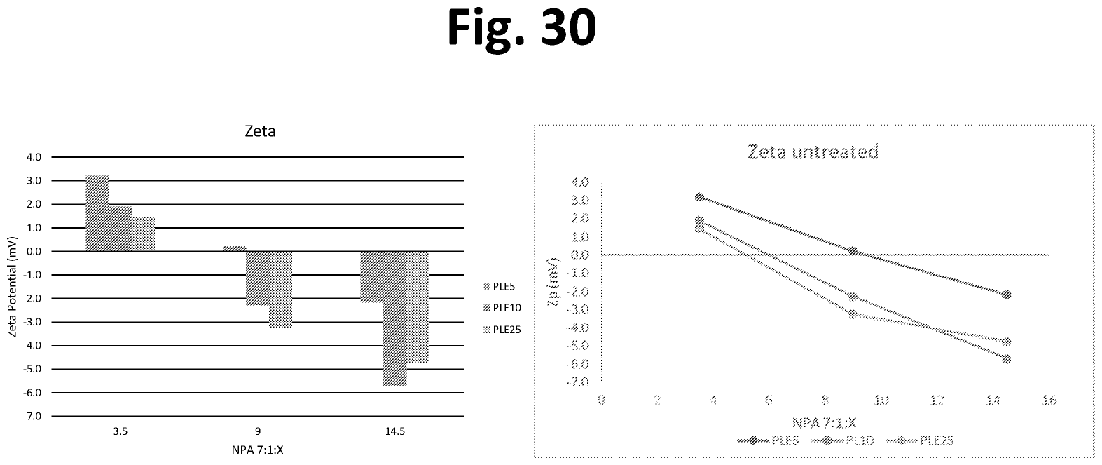

The resulting PEGylated polyplexes were assayed for particle size and no significant changes in size were identified among the prepared polyplexes. FIG. 28. The polyplexes were dispersed in water and the pH was measured, indicating that larger PLE length and decreasing N:A ratio both contributed to a higher pH of the aqueous dispersion. FIG. 29. Similarly, zeta potential measurements indicated that a higher length PLE anchor region and a decreasing N:A ratio both decreased zeta potential. FIG. 30 (left). Moreover, the inventors observed a leveling off of this trend of decreasing zeta potential for PLE25 polyplexes as N:P:A, is adjusted from 7:1:X, X=9 to 7:1:X, X=14.5, suggesting that maximum PEGylation is achieved at a lower amount of polymer. FIG. 30 (right).

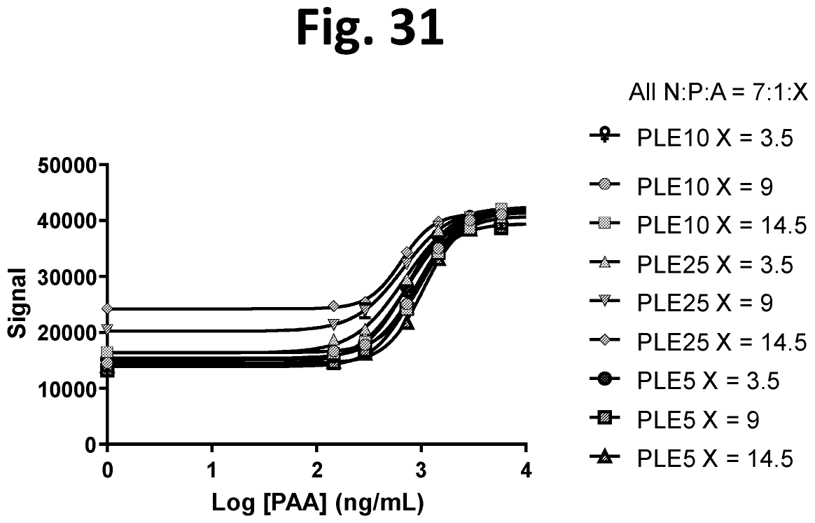

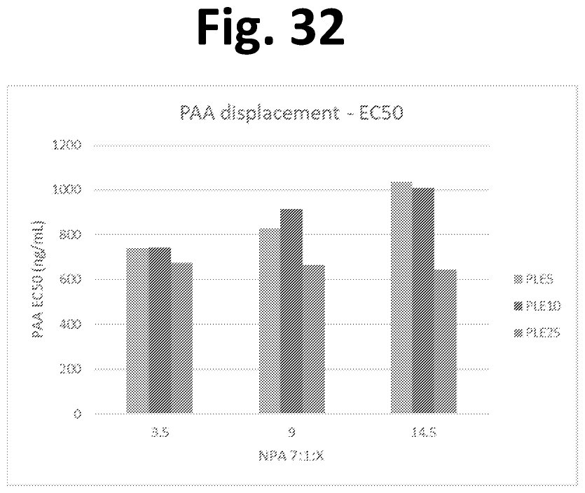

The PEGylated polyplexes were assayed in a polyaspartic acid (PAA) competition assay in which PAA was mixed in an aqueous buffer containing the PEGylated polyplexes at various concentrations and nucleic acid accessibility is observed. Nucleic acid accessibility is measured by picogreen assay. Accessible nucleic acid binds to picogreen and the increase in fluorescence signal caused by the binding is detected. Fully released and/or fully accessible nucleic acid provides a maximal fluroescence signal. The EC50 for PAA concentration required to achieve half-maximal signal indicates how easily a particle composition is disrupted by PAA contact, a measure of the stability of the tested polyplexes. The results indicate that PLE25 PEGylated polplexes are somewhat less stable than other polyplexes. FIGS. 31-32.

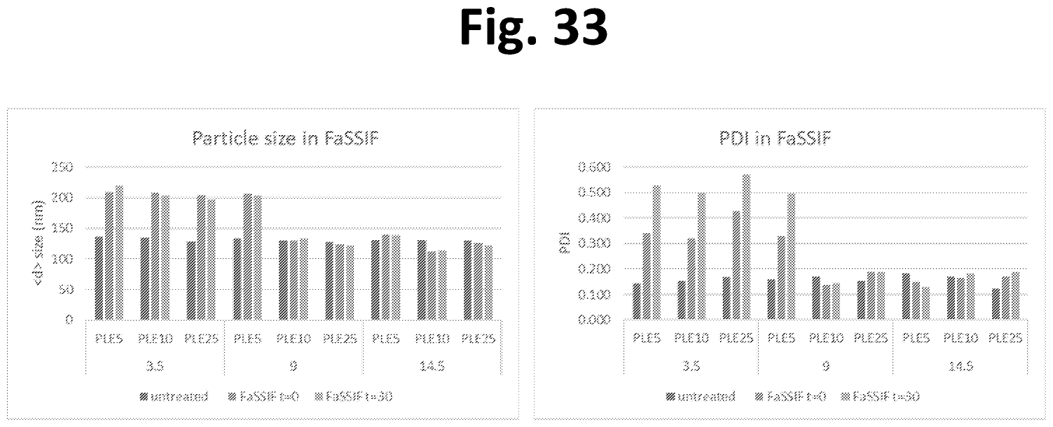

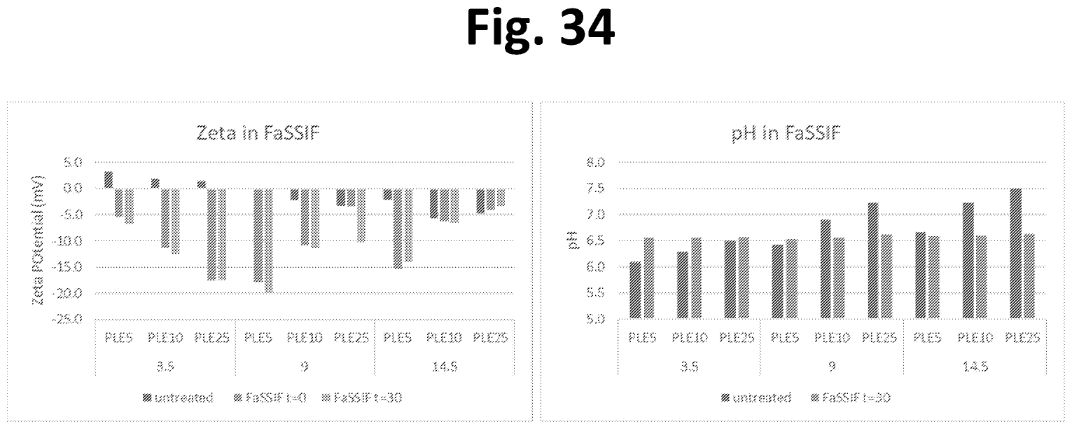

The PEGylated polyplexes were prepared as a c125 (125 μg/mL DNA) reaction mixture and then mixed with FaSSIF-V2 at a 1:2 volume ratio (excess FaSSIF). After mixing with FaSSIF-V2, DLS measurements were taken at time zero (t0) and 30 minutes (t=30) at 37° C. to measure particle size and polydispersity (FIG. 33) and the samples were also assayed for zeta potential and pH (FIG. 34). Under the conditions tested, polyplexes were more stable with longer polyanion chain lengths (e.g., PLE25 most stable PLE5 least stable) and higher amino-functionalization of chitosan (e.g., N:P:A 14.5 most stable N:P:A 3.5 least stable).

### Example 6

The effects of polyanion species and molecular weight (MW) on PEGylated polyplexes were examined. PLE (polyglutamate), PLD (plyaspartic acid, i.e., PAA), and HA (hyaluronic acid) were studied. A trehalose solution of 4.51% was used as storage stability agent. PEG polyanion work solutions were prepared.

PEG-HA 25 did not dissolve in the trehalose diluent, and into a gel-like specie. Diluted 100×, the gel did not dissolve. PEG-HA 25 was omitted from further analysis. The resulting c250 solutions were mixed with PEG polyanion at 1:1 v/v ratio by adding PEG polyanion solution to the c250 solution dropwise while vortexing. After PEG polyanion was added, vortexing was continued for 10 seconds. The following PEGylated polyplexes were produced.

| Polyplex | DNA:Anion | DNA:Anion | DNA:Anion | DNA:Anion | |
|---|---|---|---|---|---|
| Sample | Ratio | Ratio | Ratio | Ratio | |
| Name | (PA ratio) | (PA ratio) | (PA ratio) | (PA ratio) | |
| PEG-PLE10 | A4 | A3 | A2 | A1 | |
| | 1:3.5 | 1:9 | 1:15 | 1:30 | |
| PEG-PLE10 | 27 | 32 | 30 | 30 | |
| Additiona | | | | | |
| Duration (s) | | | | | |
| PEG-PLD10 | B4 | B3 | B2 | B1 | |
| | 1:3.5 | 1:9 | 1:15 | 1:30 | |
| PEG-PLD 10 | 30 | 34 | 40 | 31 | |
| Addition | | | | | |
| Duration (s) | | | | | |
| PEG-PLD 50 | C4 | C3 | C2 | C1 | |
| | 1:3.5 | 1:9 | 1:15 | 1:30 | |
| PEG-PLD 50 | 29 | 27 | 31 | 25 | |
| Addition | | | | | |
| Duration (s) | | | | | |
| PEG-HA24 | D4 | D3 | D2 | D1 | Not tested due to |
| | 1:35 | 1:9 | 1:15 | 1:30 | insoluble PEG- |
| | | | | | polyanion solution |

The above compositions were incubated at ambient temperature for 1 hr before further analysis.

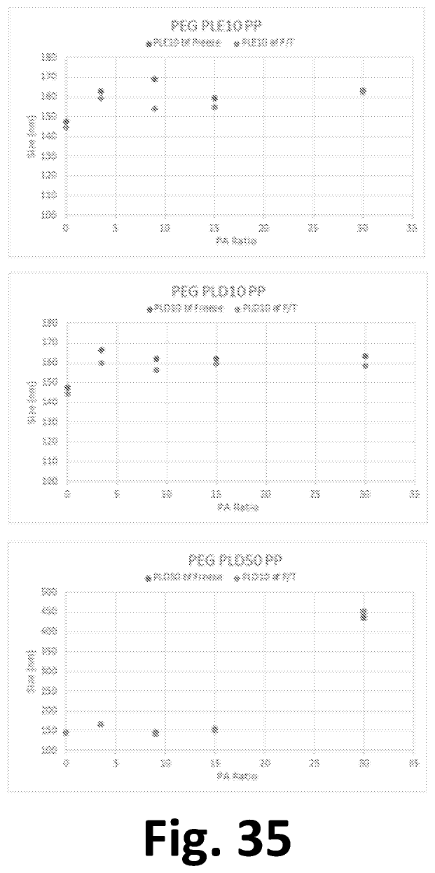

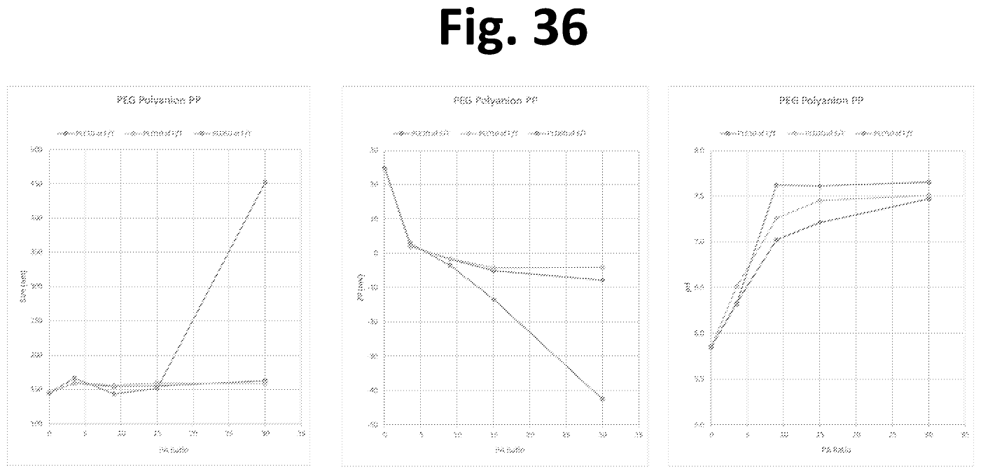

Separately, a solution of dually derivatized chitosan nucleic acid polyplexes was diluted from 1000 μg/mL nucleic acid concentration (c1000) to 125 μg/mL (c125) nucleic acid concentration in a 4.51% trehalose solution. After freeze thaw (F/T) all samples were tested for appearance, pH, size, zeta potential, and free DNA content. Results are shown in the tables below and FIGS. 35-36.

| | | Z-Ave (d.nm) | | | | Pdl | | | | Derived Count Rate (kcps) | | | |
|---|---|---|---|---|---|---|---|---|---|---|---|---|---|
| | | Before | | | | Before | | | | Before | | | |
| Sample | PA | Freezing | | After F/T | | Freezing | | After F/T | | Freezing | | After F/T | |
| Name | Ratio | AVE | STD | AVE | STD | AVE | STD | AVE | STD | AVE | STD | AVE | STD |
| non- | 0 | 148.1 | 1.5 | 144.3 | 2.0 | 0.155 | 0.019 | 0.176 | 0.007 | 11591 | 63 | 11608 | 103 |
| PEG | | | | | | | | | | | | | |
| control | | | | | | | | | | | | | |
| non- | 0 | 146.9 | 3.4 | 144.8 | 0.4 | 0.166 | 0.004 | 0.168 | 0.016 | 9857 | 184 | 9602 | 119 |
| PEG | | | | | | | | | | | | | |
| control | | | | | | | | | | | | | |
| non- | 0 | 147.5 | | 144.6 | | 0.2 | | 0.2 | | 10723.7 | | 10605.1 | |
| PEG | | | | | | | | | | | | | |
| control | | | | | | | | | | | | | |
| PEG- | 30 | 163.5 | 2.6 | 162.5 | 2.2 | 0.100 | 0.010 | 0.095 | 0.005 | 27922 | 128 | 27330 | 452 |
| PLE 10 | | | | | | | | | | | | | |
| PEG- | 15 | 159.6 | 3.2 | 155.1 | 0.8 | 0.167 | 0.017 | 0.162 | 0.008 | 22546 | 226 | 21881 | 79 |
| PLE 10 | | | | | | | | | | | | | |
| PEG- | 9 | 169.2 | 2.9 | 154.3 | 2.3 | 0.225 | 0.011 | 0.168 | 0.018 | 21287 | 163 | 19835 | 104 |
| PLE 10 | | | | | | | | | | | | | |
| PEG- | 3.5 | 162.8 | 1.6 | 159.2 | 1.7 | 0.133 | 0.009 | 0.136 | 0.015 | 17126 | 144 | 16857 | 146 |
| PLE 10 | | | | | | | | | | | | | |
| PEG- | 30 | 163.2 | 4.8 | 158.7 | 0.8 | 0.141 | 0.018 | 0.104 | 0.001 | 19261 | 463 | 19010 | 174 |
| PLD 10 | | | | | | | | | | | | | |
| PEG- | 15 | 162.2 | 3.5 | 159.3 | 2.1 | 0.156 | 0.006 | 0.159 | 0.009 | 15968 | 39 | 16965 | 103 |
| PLD 10 | | | | | | | | | | | | | |
| PEG- | 9 | 162.1 | 4.6 | 156.2 | 1.6 | 0.173 | 0.006 | 0.169 | 0.009 | 14540 | 141 | 15066 | 54 |
| PLD 10 | | | | | | | | | | | | | |
| PEG- | 3.5 | 166.7 | 3.3 | 159.7 | 0.8 | 0.156 | 0.008 | 0.156 | 0.018 | 13290 | 22 | 13775 | 43 |
| PLD 10 | | | | | | | | | | | | | |
| PEG- | 30 | 436.3 | 6.7 | 452.0 | 3.8 | 0.260 | 0.013 | 0.281 | 0.009 | 5190 | 40 | 3858 | 89 |
| PLD 50 | | | | | | | | | | | | | |
| PEG- | 15 | 154.7 | 3.2 | 151.5 | 0.2 | 0.170 | 0.016 | 0.164 | 0.003 | 12895 | 264 | 13726 | 108 |
| PLD 50 | | | | | | | | | | | | | |
| PEG- | 9 | 146.8 | 1.7 | 143.3 | 1.0 | 0.172 | 0.005 | 0.180 | 0.011 | 20111 | 315 | 21970 | 92 |
| PLD 50 | | | | | | | | | | | | | |
| PEG- | 3.5 | 165.4 | 2.4 | 166.2 | 0.4 | 0.169 | 0.008 | 0.145 | 0.004 | 18864 | 208 | 19323 | 142 |
| PLD 50 | | | | | | | | | | | | | |

| | | ZP (mV) | | | | Cond (mS/cm) | | | |
|---|---|---|---|---|---|---|---|---|---|
| | PA | Before Freezing | | After F/T | | Before Freezing | | After F/T | |
| Sample Name | Ratio | AVE | STD | AVE | STD | AVE | STD | AVE | STD |
| non-PEG control | 0 | 28.3 | 1.3 | 24.6 | 0.8 | 1.120 | 0.040 | 1.133 | 0.040 |
| non-PEG control | 0 | 27.8 | 0.4 | 24.9 | 1.1 | 1.113 | 0.040 | 1.113 | 0.040 |
| non-PEG control | 0 | 28.0 | | 24.8 | | 1.1 | | 1.1 | |
| PEG-PLE 10 | 30 | −7.3 | 1.0 | −7.9 | 0.5 | 1.207 | 0.045 | 1.220 | 0.046 |
| PEG-PLE 10 | 15 | −5.6 | 0.5 | −5.1 | 0.3 | 1.180 | 0.046 | 1.133 | 0.040 |
| PEG-PLE 10 | 9 | −1.5 | 0.1 | −1.8 | 0.0 | 1.163 | 0.040 | 1.113 | 0.040 |
| PEG-PLE 10 | 3.5 | 2.0 | 0.3 | 2.1 | 0.8 | 1.123 | 0.040 | 1.143 | 0.040 |
| PEG-PLD 10 | 30 | −4.8 | 0.5 | −4.2 | 0.4 | 1.197 | 0.045 | 1.203 | 0.050 |
| PEG-PLD 10 | 15 | −3.5 | 0.4 | −4.3 | 0.3 | 1.153 | 0.040 | 1.187 | 0.045 |
| PEG-PLD 10 | 9 | −1.4 | 0.3 | −1.5 | 0.2 | 1.137 | 0.045 | 1.147 | 0.045 |
| PEG-PLD 10 | 3.5 | 1.7 | 0.1 | 1.8 | 0.1 | 1.123 | 0.040 | 1.123 | 0.040 |
| PEG-PLD 50 | 30 | −43.1 | 1.0 | −42.5 | 2.0 | 1.150 | 0.046 | 1.153 | 0.040 |
| PEG-PLD 50 | 15 | −14.8 | 0.7 | −13.5 | 0.4 | 1.143 | 0.040 | 1.160 | 0.046 |
| PEG-PLD 50 | 9 | −4.5 | 0.2 | −3.5 | 0.1 | 1.187 | 0.045 | 1.133 | 0.040 |
| PEG-PLD 50 | 3.5 | 3.3 | 0.3 | 2.9 | 0.2 | 1.103 | 0.040 | 1.113 | 0.040 |

| | | pH of | | |
|---|---|---|---|---|
| | | Stock | | |
| | | Solution | pH | pH |
| | PA | PEG | Before | After |
| Sample Name | Ratio | polyanion | Freezing | F/T |
| non-PEG control | 0 | ND | 5.84 | 5.66 |
| non-PEG control | 0 | ND | 5.73 | 6.04 |
| non-PEG control | 0 | ND | 5.8 | 5.9 |
| PEG-PLE 10 | 30 | 7.35 | 8.07 | 7.47 |
| PEG-PLE 10 | 15 | 6.88 | 7.83 | 7.21 |
| PEG-PLE 10 | 9 | 6.74 | 7.71 | 7.02 |
| PEG-PLE 10 | 3.5 | 6.32 | 7.48 | 6.33 |
| PEG-PLD 10 | 30 | 7.09 | 7.95 | 7.51 |
| PEG-PLD 10 | 15 | 6.82 | 7.05 | 7.45 |
| PEG-PLD 10 | 9 | 7.07 | 7.30 | 7.26 |
| PEG-PLD 10 | 3.5 | 6.39 | 6.69 | 6.51 |
| PEG-PLD 50 | 30 | 7.21 | 7.73 | 7.65 |
| PEG-PLD 50 | 15 | 7.05 | 7.50 | 7.61 |
| PEG-PLD 50 | 9 | 7.36 | 7.55 | 7.62 |
| PEG-PLD 50 | 3.5 | 6.27 | 7.12 | 6.32 |

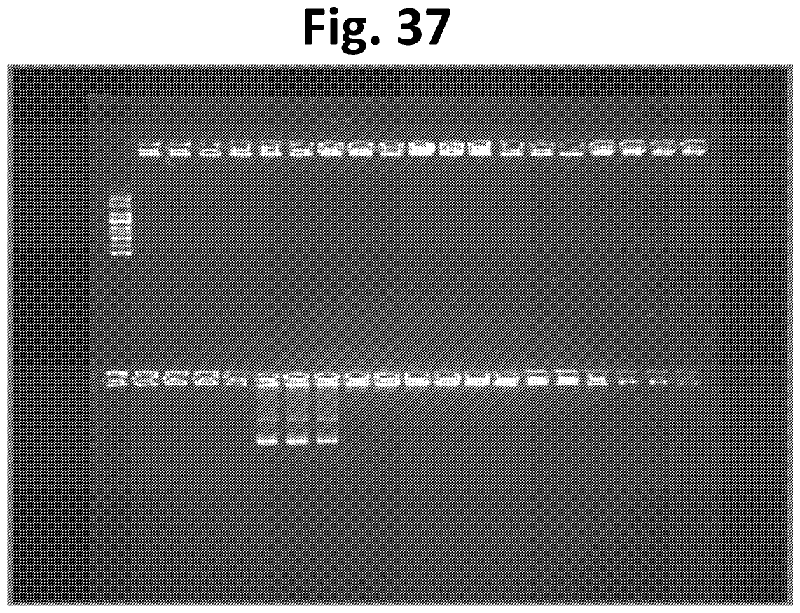

Polyplex samples were analyzed by agarose gel electrophoresis to detect uncomplexed nucleic acid. FIG. 37. All samples tested, except for 7:1:30 N:P:A polyplexes PEGylated with PEG-PLD50, (FIG. 37, botton rows 6-8), exhibited no detectable uncomplexed nucleic acid.

The results suggest the following. PEG-PLE10, PEG-PLD10, and PEG-PLD50 are compatible with DDX polyplex at 7:1 NP ratio for PEGylation. Post F/T, no precipipitate was observed in any of the c125 PEGylated polyplex solutions. PEG-PLE10 and PEG-PLD10 behave similarly when mixing with polyplex. Size of polyplex increased from 140 nm to 160 nm post PEGylation. And the size plateaued at 160 nm for NPA ratio of 7:1:3.5, 7:1:5, 7:1:9, 7:1:15 and 7:1:30. Zeta potential decreases as PEG polyanion ratio increases, but the decrease slowed down beyond 7:1:9, suggesting the PEGylation may be reaching saturation. For PEG-PLD50, post PEGylation, up to NPA ratio of 7:1:15, the polyplex size is comparable with the polyplex before PEGylation (7:1:0). At NPA 7:1:30, the polyplex size increased to 440 nm post PEGylation. Zeta potential of PEG-PLD50 PEGylated polyplex decreases as the PEG-PLD50 ratio increases. Free DNA was observed for 7:1:30 with PEG-PLD50 while no free DNA visible for the other formulations, suggesting the nucleic acid in the polyplex of 7:1:30 PEG-PLD50 is loosely wrapped and/or PLD-50 disrupts binding of derivatized chitosan to nucleic acid.

### Example 6

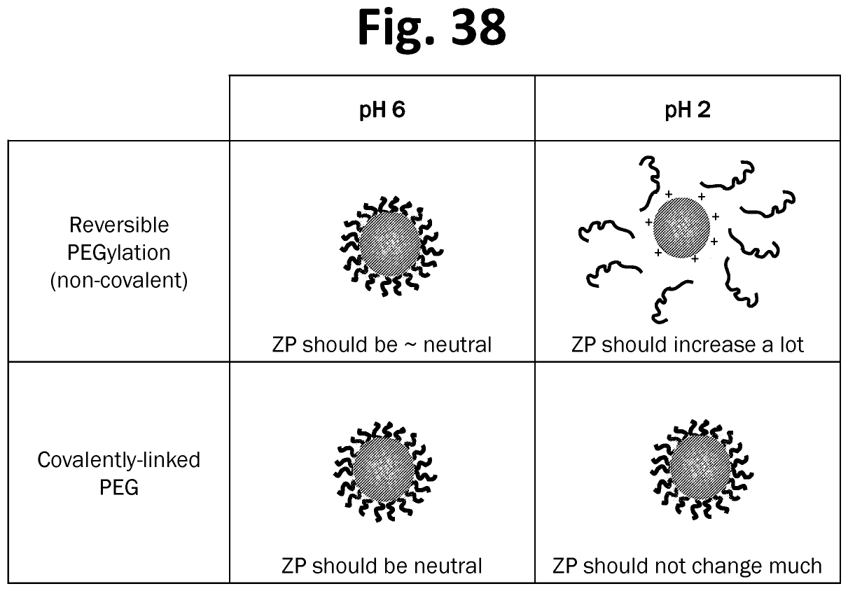

The effect of non-covalent reversible PEGylation was compared to covalent PEGylation. As illustrated in FIG. 38, non-covalently PEGylated polyplexes having a titratable polyanion anchoring region and covalently PEGylated polyplexes are both expected to have a zeta potential near neutral under pH conditions that maintain the negative charge of the polyanionic anchoring region. Upon titration of the polyanion anchoring region to a neutral or positive charge at low pH (e.g., pH 2), zeta potential of the non-covalently PEGylated polyplexes should detectably increase. In contrast, covalently-linked PEG cannot be released and zeta potential should not change as much.

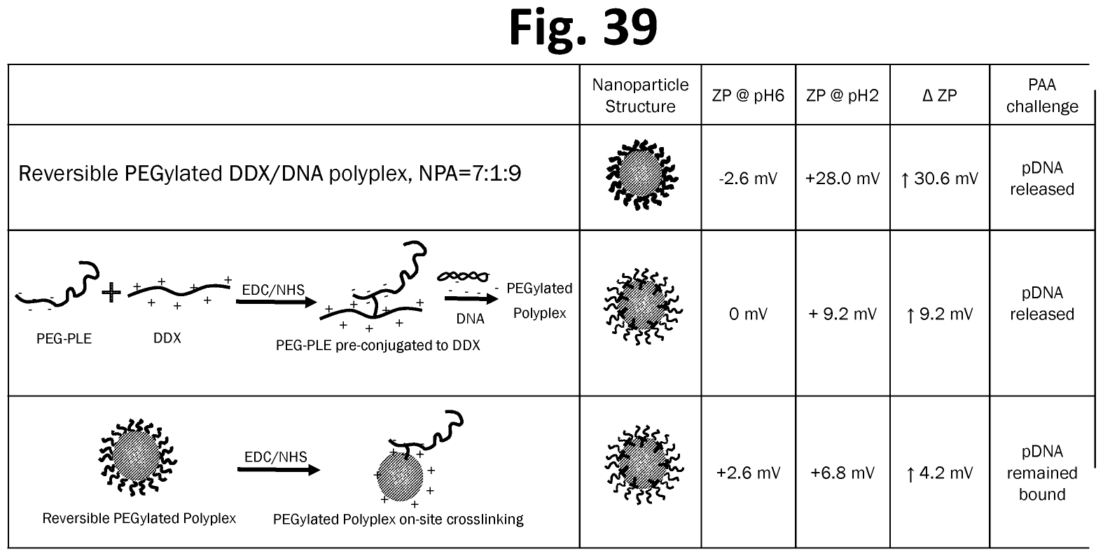

Two different covalent PEGylation strategies were studied as shown in FIG. 39. In the first, PEG-PLE was cross-linked to dually-derivatized (DDX) chitosan to form a cross-linked PEG-chitosan feedstock and DNA was added to form polyplex (PEG crosslinked to chitosan). (FIG. 39, middle row). In the second, reversibly PEGylated polyplexes were formed and EDC/NHS was added to covalently cross-link the PEG-PA polymers to the polyplex (PEG crosslinked to polyplex). (FIG. 39, bottom). Both of the covalently PEGylated polyplexes showed minimal change in zeta potential after adjusting the pH from 6 to 2. In contrast, the zeta potential of the reversibly PEGylated polyplexes increased significantly. The polyplexes were also tested for stability by challenge with free polyaspartic acid (PAA). The nucleic acid in the polyplexes was released by PAA challenge from reversibly PEGylated polyplexes and the PEG cross-linked to chitosan polyplexes. In contrast, cross-linking after polyplex formation provided polyplexes that did not release nucleic acid after PAA challenge under the conditions tested.

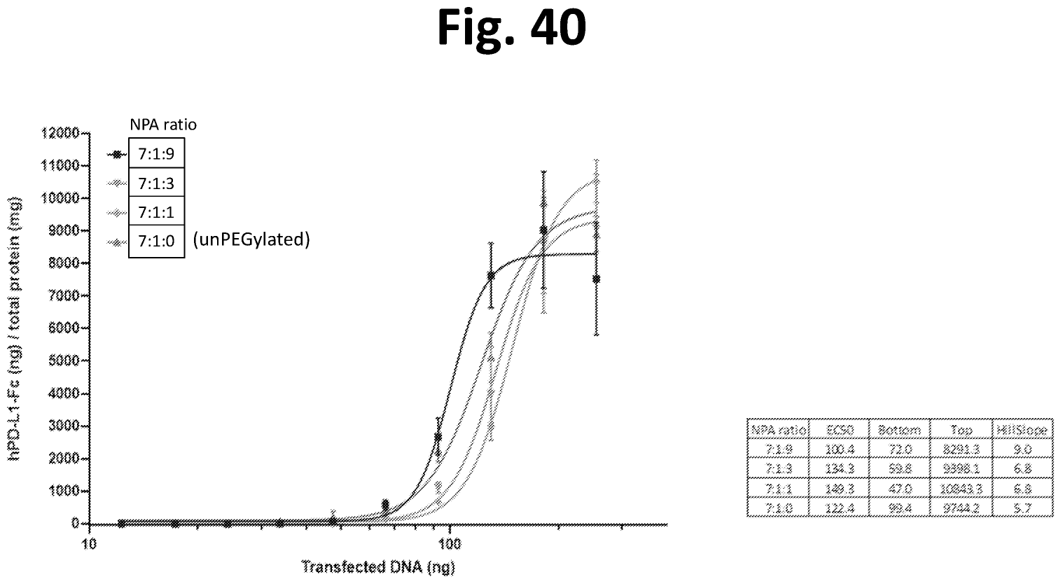

Tranfection efficiency was assayed for reversibly PEGylated polyplexes at N:P:A 7:1:X, where X is 9, 3, or 1, and compared with unPEGylated polyplexes (7:1:0). The results indicated that transfection efficiency was not diminished by reversible PEGylation. FIG. 40.

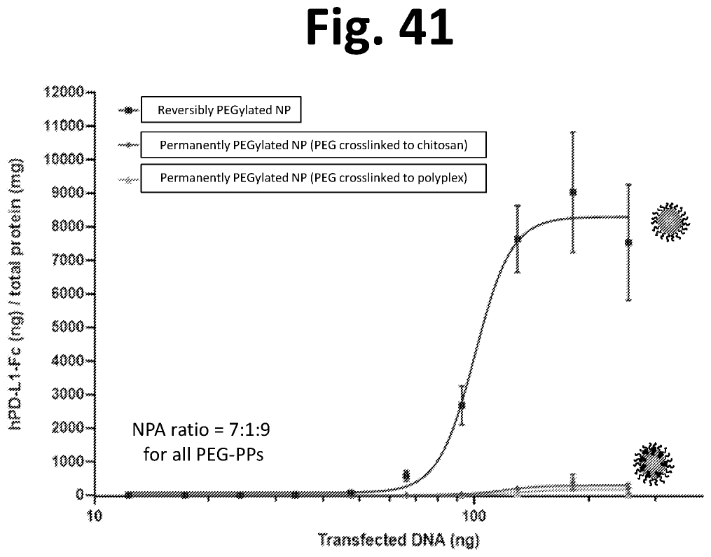

Transfection efficiency of reversibly PEGylated and covalently PEGylated polyplexes were compared. As illustrated in FIG. 41, reversibly PEGylated polyplexes retained high transfection efficiency, whereas both types of covalently PEGylated polyplexes showed poor transfection efficiency.

### Example 7

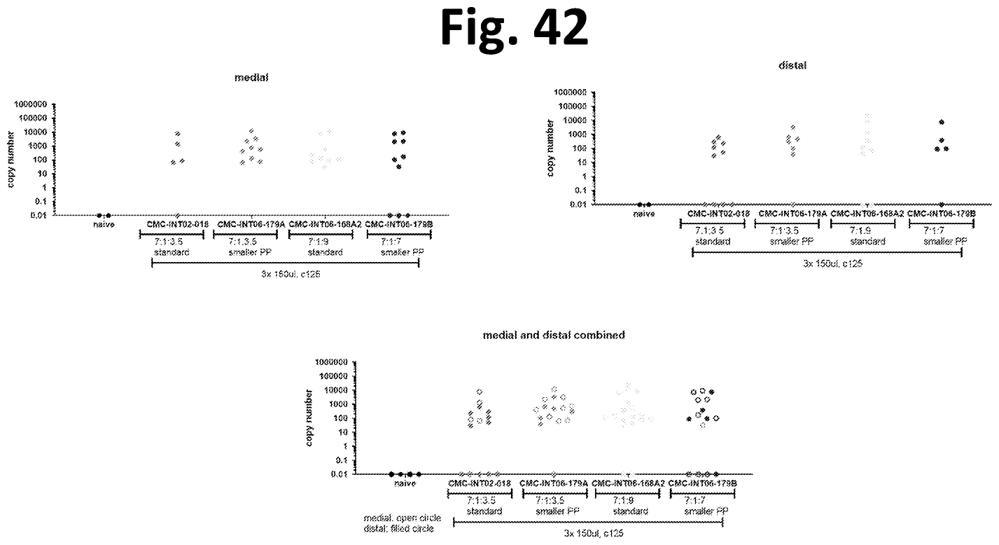

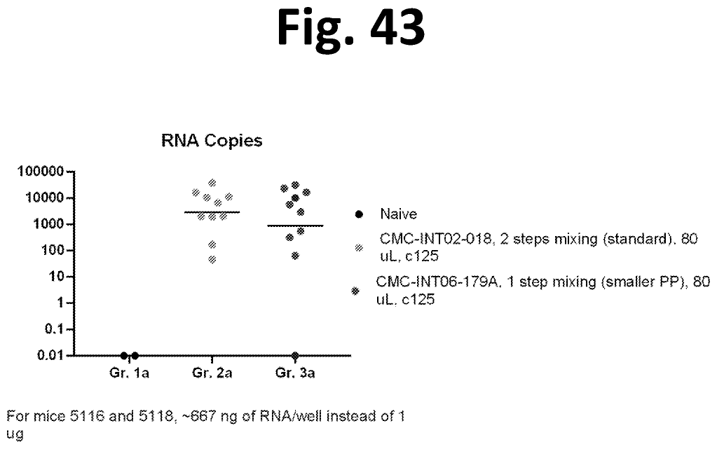

Transfection efficiencies of reversibly PEGylated polyplexes formed by one-step mixing of nucleic acid, chitosan, and PEG-polyanion polymer (one-step) were compared to reversibly PEGylated polyplexes formed by mixing preformed chitosan DNA polyplexes with PEG-polyanion polymer (two-step). mRNA expression was detected in colon tissue 24 h after intracolonic delivery of one-step or two-step PEGylated polyplexes. mRNA and protein was detected in bladder tissue 24 h after intravesicular delivery of one-step or two-step PEGylated polyplexes. The results, shown in FIGS. 42-43, indicate that both one-step polyplexes and two-step polyplexes exhibit similar transfection efficiencies under the conditions tested. Without wishing to be bound by theory, it is hypothesized that the one-step polyplexes, which are typically smaller in size, may exhibit superior diffusion properties in highly viscous mucosa.

### EQUIVALENTS

All publications, patents, and patent applications mentioned in this specification are herein incorporated by reference in the entirety and for all purposes and to the same extent as if each individual publication, patent, or patent application was specifically and individually indicated to be incorporated by reference. The disclosure set forth above may encompass multiple distinct inventions with independent utility. Although each of these inventions has been disclosed in its preferred form(s), the specific embodiments thereof as disclosed and illustrated herein are not to be considered in a limiting sense, because numerous variations are possible. The subject matter of the inventions includes all novel and nonobvious combinations and subcombinations of the various elements, features, functions, and/or properties disclosed herein. The following claims particularly point out certain combinations and subcombinations regarded as novel and nonobvious. Inventions embodied in other combinations and subcombinations of features, functions, elements, and/or properties may be claimed in this application, in applications claiming priority from this application, or in related applications. Such claims, whether directed to a different invention or to the same invention, and whether broader, narrower, equal, or different in scope in comparison to the original claims, also are regarded as included within the subject matter of the inventions of the present disclosure.

1. A composition comprising:
(a) a complex comprising a chitosan-derivative nanoparticle comprising amino-functionalized chitosan and at least one nucleic acid molecule, wherein the at least one nucleic acid molecule is non-covalently bound to the chitosan-derivative nanoparticle at an amino to phosphorous (N:P) molar ratio of greater than 3:1, thereby forming a derivatized chitosan nucleic acid complex having a positive charge; and
(b) a plurality of linear block copolymers non-covalently bound to the chitosan-derivative nanoparticle, wherein said linear block copolymers comprise at least one polyanionic (PA) anchor region and at least one polyethylene glycol (PEG) tail region, wherein the PEG-PA molecules are non-covalently bound to the chitosan-derivative nanoparticle, and
wherein the composition comprises an amino to anion (N:A) molar ratio that is greater than about 1:100 and less than about 10:1.

2. The composition according to claim 1, wherein said linear block copolymer is a diblock copolymer comprising a PA anchor region and a PEG tail region.

3. The composition according to claim 1, wherein said linear block copolymer is a triblock copolymer comprising a central PA anchor region flanked by two PEG tail regions, or alternatively a central PEG tail region flanked by two PA anchor regions.

4. The composition according to claim 1, wherein the PA tail region comprises a polypeptide, wherein the polypeptide is negatively charged.

5. The composition according to claim 1, wherein the PA tail region comprises a carbohydrate, wherein the carbohydrate is negatively charged.

6. The composition according to claim 5, wherein the carbohydrate comprises a plurality of carboxylate, phosphate, and/or sulfate moieties.

7. The composition according to claim 6, wherein the carbohydrate is a glycosaminoglycan.

8. The composition according to claim 1, wherein PEG-PA molecules comprise:
(a) PEG-polyglutamic acid (PEG-PGA) molecules;
(b) PEG-polyaspartic acid (PEG-PAA) molecules; or
(c) PEG-hyaluronic acid (PEG-HA) molecules,
or a combination of 1, or 2, or all of (a)-(c).

9. The composition according to claim 1, wherein the PEG portion of the PEG-PA molecules comprise a weight average molecular weight (Mw) of from about 500 Da to about 50,000 Da, preferably from about 1,000 Da to about 10,000 Da, more preferably from about 1,500 Da to about 7,500 Da, yet more preferably from about 3,000 Da to about 5,000 Da, most preferably about 5,000 Da.

10. The composition according to claim 1, wherein the PA portion of the PEG-PA molecules comprise a weight average molecular weight (Mw) of from about 500 Da to about 3,000 Da, more preferably from about 1,000 Da to about 2,500 Da, more preferably about 1,500 Da.

11. The composition according to claim 1, wherein the PA portion of the PEG-PA molecules comprises a, e.g., linear, polypeptide comprising from about 5 to about 25 acidic amino acids.

12. The composition according to claim 1, wherein the N:P molar ratio is greater than about 3:1 and less than about 100:1, more preferably greater than about 5:1 and less than about 50:1, yet more preferably greater than about 5:1 and less than about 30:1, yet more preferably greater than about 5:1 and less than about 20:1, yet more preferably greater than about 5:1 and less than about 10:1, most preferably about 7:1.

13. The composition according to claim 1, wherein the N:A molar ratio is greater than about 1:75 and less than about 8:1, more preferably greater than about 1:50 and less than about 6:1, yet more preferably greater than about 1:25 and less than about 6:1, yet more preferably greater than about 1:10 and less than about 6:1, yet more preferably greater than about 1:5 and less than about 6:1.

14. The composition according to claim 1, wherein the N:P molar ratio is from about 1:8 to about 30:1 (e.g., to about 20:1, 15:1, 10:1, 8:1, or 7:1), and wherein the P:A molar ratio is from about 1:50 to about 1:5, more preferably wherein the N:A molar ratio is from about 1:10 to about 5, more preferably from about 1:5 to about 2, more preferably from about 1:3 to about 1.5, more preferably from about 1:2.5 to about 1, yet more preferably wherein the N:P:A ratio is about 7:1:7; about 7:1:12; or about 7:1:17.

15. The composition according to claim 1, wherein the chitosan-derivative nanoparticle comprises a polyol of Formula II or is functionalized with a polyol of Formula II: 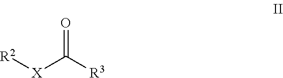
wherein
(a) R² is selected from: H and hydroxyl;
(b) R³ is selected from: H and hydroxyl; and
(c) X is selected from C₂-C₆ alkylene optionally substituted with one or more hydroxyl substituents.

16. The composition according to claim 1, wherein the chitosan-derivative nanoparticle comprises a polyol of Formula III: 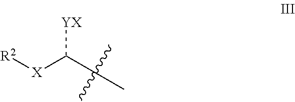
wherein:
—Y is ═O or —H₂;
R² is selected from: H and hydroxyl;
R³ is selected from: H and hydroxyl;
X is selected from: C₂-C₆ alkylene optionally substituted with one or more hydroxyl substituents; and 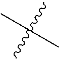
denotes the bond between the polyol and the derivatized chitosan.

17. The composition according to claim 1, wherein amino-functionalized chitosan is arginine, lysine, or ornithine functionalized, preferably arginine functionalized.

18. The composition according to claim 1, wherein the composition is stable:
(a) for at least 24 hours in fasted state simulated intestinal fluid; or
(b) for at least 1 h dispersed in mammalian urine at 37° C.

19. The composition according to claim 1, wherein the composition further comprises a surfactant, excipient, and/or a storage stability agent.

20. The composition according to claim 19, wherein the composition comprises the storage stability agent, preferably wherein the storage stability agent is a monosaccharide, a disaccharide, a polysaccharide, or a reduced alcohol thereof, yet more preferably wherein the storage stability agent is selected from trehalose and mannitol.

21. The composition according to claim 19, wherein the composition comprises the surfactant, preferably wherein the surfactant comprises a poloxamer, more preferably wherein the poloxamer is poloxamer 407.

22. The composition according to claim 1, wherein the at least one nucleic acid comprises RNA.

23. The composition according to claim 1, wherein the at least one nucleic acid comprises DNA.

24. The composition according to claim 1, wherein the composition is stable for, or for at least, 48 h, or 1 week at 4° C. in an aqueous dispersion comprising the composition dispersed in purified water.

25. The composition according to claim 24, wherein the composition is stable in the aqueous dispersion after freeze/thaw and/or drying/rehydration, preferably wherein the drying comprises spray drying, lyopholization, spray freeze drying, evaporation, or supercritical drying, more preferably wherein the drying comprises lyopholization or spray drying.

26. The composition according to claim 24, wherein the composition exhibits a polydispersity index of less than 0.2 after, at least, 48 h, or 1 week at 4° C. in the aqueous dispersion.

27. A method for making the composition of claim 1, the method comprising:
(a) providing a complex comprising the chitosan-derivative nanoparticle comprising amino-functionalized chitosan and the at least one nucleic acid; and
(b) mixing the complex with a solution comprising PEG-PA molecules, thereby forming a reaction mixture comprising the composition.

28. The method of claim 27, wherein the complex of (a) is provided at a nucleotide concentration of from 0.01 mg/mL to 25 mg/mL, more preferably from 0.05 to 10 mg/mL, more preferably from 0.10 to 5 mg/mL, more preferably from 0.10 to 2 mg/mL.

29. The method of claim 27, wherein (a) and (b) are mixed at a (v/v) ratio of from 1:10 to 10:1, preferably from 1:5 to 5:1, more preferably from 1:2 to 2:1, yet more preferably at a ratio of about 1:1.

30. The method of claim 27, wherein the method further comprises concentrating the reaction mixture, preferably the concentrating comprises ultrafiltration, solvent sublimation, and/or solvent evaporation, more preferably the ultrafiltration comprises tangential flow filtration.

31. The method of claim 27, wherein the amino-functionalized chitosan comprises or is functionalized with a hydrophilic polyol.

32. A method for making the composition of claim 1, the method comprising simultaneously or sequentially admixing amino-functionalized chitosan, at least one nucleic acid, and PEG-PA molecules, thereby forming a reaction mixture comprising the composition.

33. The method of claim 32, wherein the method comprises simultaneously combining the amino-functionalized chitosan, at least one nucleic acid, and the PEG-PA molecules, thereby forming a reaction mixture comprising the composition.

34. A method of transfecting a cell with nucleic acid comprising contacting the cell with a composition according to claim 1 or a composition produced by a method of claim 27 or 32.

35. The method of claim 34, wherein the cell is a cell comprising or derived from a mucosal tissue.

36. The method of claim 35, wherein the mucosal tissue is lung tissue, nasal tissue, ocular tissue, vaginal tissue, bladder tissue, or gastrointestinal tract tissue.

37. The method of claim 35, wherein the cell is an intestinal cell of a subject and the contacting comprises orally or rectally administering the composition to the subject.

38. The method of claim 35, wherein the cell is a cell of the bladder and the contacting comprises intravesical administration of the composition to the subject.

39. The method of claim 27, wherein the method provides decreased muco-adhesion as compared to particles that do not comprise the polymer component comprising PEG-PA.
